(19) United States
(12) Patent Application Publication
(10) Pub. No.: US 2026/0078113 A1
(43) Pub. Date: Mar. 19, 2026

(54) HETEROCYCLIC COMPOUNDS AS IMMUNOMODULATORS

(71) Applicant: Incyte Corporation, Wilmington, DE (US)

(72) Inventors: Liangxing Wu, Wilmington, DE (US); Zhiyong Yu, Wilmington, DE (US); Fenglei Zhang, Berwyn, PA (US); Wenqing Yao, Chadds Ford, PA (US)

(21) Appl. No.: 19/400,272

(22) Filed: Nov. 25, 2025

Related U.S. Application Data
(63) Continuation of application No. 17/125,624, filed on Dec. 17, 2020, now Pat. No. 12,516,044.
Application No. 17/125,624 is a continuation of application No. 16/870,436, filed on May 8, 2020, now abandoned.
Application No. 16/870,436 is a continuation of application No. 16/580,799, filed on Sep. 24, 2019, now abandoned.
Application No. 16/580,799 is a continuation of application No. 16/266,436, filed on Feb. 4, 2019, now abandoned.
Application No. 16/266,436 is a continuation of application No. 16/008,124, filed on Jun. 14, 2018, now abandoned.
Application No. 16/008,124 is a continuation of application No. 15/802,850, filed on Nov. 3, 2017, now abandoned.
Application No. 15/802,850 is a continuation of application No. 15/381,370, filed on Dec. 16, 2016, now abandoned.
(60) Provisional application No. 62/268,680, filed on Dec. 17, 2015.
(60) Provisional application No. 62/347,817, filed on Jun. 9, 2016.
(60) Provisional application No. 62/385,048, filed on Sep. 8, 2016.

(51) Int. Cl. C07D 413/12; C07D 213/81; C07D 213/84; C07D 237/24; C07D 239/28; C07D 241/24; C07D 401/06; C07D 401/12; C07D 401/14; C07D 405/12; C07D 405/14

(52) U.S. Cl. CPC C07D 413/12; C07D 213/81; C07D 213/84; C07D 237/24; C07D 239/28; C07D 241/24; C07D 401/06; C07D 401/12; C07D 401/14; C07D 405/12; C07D 405/14

(57) ABSTRACT

Disclosed are compounds of Formula (I′), methods of using the compounds as immunomodulators, and pharmaceutical compositions comprising such compounds. The compounds are useful in treating, preventing or ameliorating diseases or disorders such as cancer or infections.

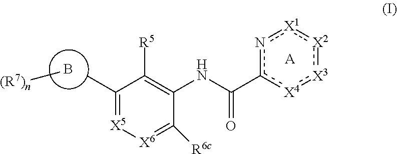

### FIELD OF THE INVENTION

The present application is concerned with pharmaceutically active compounds. The disclosure provides compounds as well as their compositions and methods of use. The compounds modulate PD-1/PD-L1 protein/protein interaction and are useful in the treatment of various diseases including infectious diseases and cancer.

### BACKGROUND OF THE INVENTION

The immune system plays an important role in controlling and eradicating diseases such as cancer. However, cancer cells often develop strategies to evade or to suppress the immune system in order to favor their growth. One such mechanism is altering the expression of co-stimulatory and co-inhibitory molecules expressed on immune cells (Postow et al, J. Clinical Oncology 2015, 1-9). Blocking the signaling of an inhibitory immune checkpoint, such as PD-1, has proven to be a promising and effective treatment modality.

Programmed cell death-1 (PD-1), also known as CD279, is a cell surface receptor expressed on activated T cells, natural killer T cells, B cells, and macrophages (Greenwald et al, Annu. Rev. Immunol 2005, 23:515-548; Okazaki and Honjo, Trends Immunol 2006, (4):195-201). It functions as an intrinsic negative feedback system to prevent the activation of T-cells, which in turn reduces autoimmunity and promotes self-tolerance. In addition, PD-1 is also known to play a critical role in the suppression of antigen-specific T cell response in diseases like cancer and viral infection (Sharpe et al, Nat Immunol 2007 8, 239-245; Postow et al, J. Clinical Oncol 2015, 1-9).

The structure of PD-1 consists of an extracellular immunoglobulin variable-like domain followed by a transmembrane region and an intracellular domain (Parry et al, Mol Cell Biol 2005, 9543-9553). The intracellular domain contains two phosphorylation sites located in an immunoreceptor tyrosine-based inhibitory motif and an immunoreceptor tyrosine-based switch motif, which suggests that PD-1 negatively regulates T cell receptor-mediated signals. PD-1 has two ligands, PD-L1 and PD-L2 (Parry et al, Mol Cell Biol 2005, 9543-9553; Latchman et al, Nat Immunol 2001, 2, 261-268), and they differ in their expression patterns. PD-L1 protein is upregulated on macrophages and dendritic cells in response to lipopolysaccharide and GM-CSF treatment, and on T cells and B cells upon T cell receptor and B cell receptor signaling. PD-L1 is also highly expressed on almost all tumor cells, and the expression is further increased after IFN-7 treatment (Iwai et al, PNAS 2002, 99(19):12293-7; Blank et al, Cancer Res 2004, 64(3):1140-5). In fact, tumor PD-L1 expression status has been shown to be prognostic in multiple tumor types (Wang et al, Eur J Surg Oncol 2015; Huang et al, Oncol Rep 2015; Sabatier et al, Oncotarget 2015, 6(7): 5449-5464). PD-L2 expression, in contrast, is more restricted and is expressed mainly by dendritic cells (Nakae et al, J Immunol 2006, 177:566-73). Ligation of PD-1 with its ligands PD-L1 and PD-L2 on T cells delivers a signal that inhibits IL-2 and IFN-7 production, as well as cell proliferation induced upon T cell receptor activation (Carter et al, Eur J Immunol 2002, 32(3):634-43; Freeman et al, J Exp Med 2000, 192(7):1027-34). The mechanism involves recruitment of SHP-2 or SHP-1 phosphatases to inhibit T cell receptor signaling such as Syk and Lck phosphorylation (Sharpe et al, Nat Immunol 2007, 8, 239-245). Activation of the PD-1 signaling axis also attenuates PKC-θ activation loop phosphorylation, which is necessary for the activation of NF-κB and AP1 pathways, and for cytokine production such as IL-2, IFN-7 and TNF (Sharpe et al, Nat Immunol 2007, 8, 239-245; Carter et al, Eur J Immunol 2002, 32(3):634-43; Freeman et al, J Exp Med 2000, 192(7):1027-34).

Several lines of evidence from preclinical animal studies indicate that PD-1 and its ligands negatively regulate immune responses. PD-1-deficient mice have been shown to develop lupus-like glomerulonephritis and dilated cardiomyopathy (Nishimura et al, Immunity 1999, 11:141-151; Nishimura et al, Science 2001, 291:319-322). Using an LCMV model of chronic infection, it has been shown that PD-1/PD-L1 interaction inhibits activation, expansion and acquisition of effector functions of virus-specific CD8 T cells (Barber et al, Nature 2006, 439, 682-7). Together, these data support the development of a therapeutic approach to block the PD-1-mediated inhibitory signaling cascade in order to augment or “rescue” T cell response. Accordingly, there is a need for new compounds that block PD-1/PD-L1 protein/protein interaction.

### SUMMARY

The present disclosure provides, inter alia, a compound of Formula (I′):

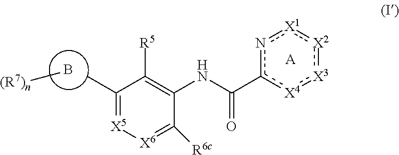

or a pharmaceutically acceptable salt or a stereoisomer thereof, wherein constituent variables are defined herein.

The present disclosure further provides, inter alia, a compound of Formula (I):

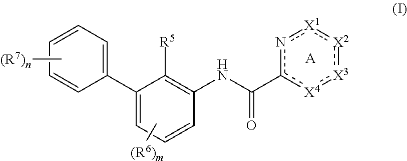

or a pharmaceutically acceptable salt or a stereoisomer thereof, wherein constituent variables are defined herein.

The present disclosure further provides a pharmaceutical composition comprising a compound of the disclosure, or a pharmaceutically acceptable salt or a stereoisomer thereof, and at least one pharmaceutically acceptable carrier or excipient.

The present disclosure further provides methods of modulating or inhibiting PD-1/PD-L1 protein/protein interaction, which comprises administering to an individual a compound of the disclosure, or a pharmaceutically acceptable salt or a stereoisomer thereof.

The present disclosure further provides methods of treating a disease or disorder in a patient comprising administering to the patient a therapeutically effective amount of a compound of the disclosure, or a pharmaceutically acceptable salt or a stereoisomer thereof.

### DETAILED DESCRIPTION

### I. Compounds

The present disclosure provides compounds of Formula (I′):

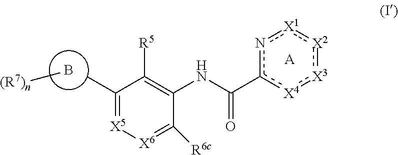

or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
X⁵ is N or CR^{6a};
X⁶ is N or CR^{6b};
ring B is C₆₋₁₀ aryl, C₃₋₁₀ cycloalkyl, 5- to 14-membered heteroaryl, or 4- to 10-membered heterocycloalkyl, provided ring B is other than 9-H-carbazol-4-yl, 2,3,4,9-tetrahydro-1H-carbazol-5-yl or 1H-tetrazolyl;
R¹, R², R³, R⁴ and R⁷ are each independently selected from H, halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1, 2, 3, or 4 R^{b} substituents;
R⁵ is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR¹¹, SR¹¹, NH₂, —NHR¹¹, —NR¹¹R¹¹, NHOR¹¹, C(O)R¹¹, C(O)NR¹¹R¹¹, C(O)OR¹¹, OC(O)R¹¹, OC(O)NR¹¹R¹¹, NR¹¹C(O)R¹¹, NR¹¹C(O)OR¹¹, NR¹¹C(O)NR¹¹R¹¹, C(═NR¹¹)R¹¹, C(═NR¹¹)NR¹¹R¹¹, NR¹¹C(═NR¹¹)NR¹¹R¹¹, NR¹¹S(O)R¹¹, NR¹¹S(O)₂R¹¹, NR¹¹S(O)₂NR¹¹R¹¹, S(O)R¹¹, S(O)NR¹¹R¹¹, S(O)₂R¹¹, and S(O)₂NR¹¹R¹¹, wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl- and (4-10 membered heterocycloalkyl)-C₁₋₄ alkyl- of R⁵ are each optionally substituted with 1, 2 or 3 R^{b} substituents; wherein R⁵ is other than F when ring B is C₃₋₁₀ cycloalkyl, 5- to 14-membered heteroaryl or 4- to 10-membered heterocycloalkyl;
each R¹¹ is independently selected from H, CN, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R¹¹ are each optionally substituted with 1, 2 or 3 R^{b} substituents;
R^{6a}, R^{6b} and R^{6c} are each independently selected from H, C₁₋₄ alkyl, C₃₋₁₀ cycloalkyl, C₃₋₁₀ cycloalkyl-C₁₋₄ alkyl-, C₆₋₁₀ aryl, C₆₋₁₀ aryl-C₁₋₄ alkyl-, 5-10 membered heteroaryl, 4-10 membered heterocycloalkyl, (5-10 membered heteroaryl)-C₁₋₄ alkyl-, (4-10 membered heterocycloalkyl)-C₁₋₄ alkyl-, C₂₋₄ alkenyl, C₂₋₄ alkynyl, halo, CN, OR¹⁰, C₁₋₄ haloalkyl, C₁₋₄ haloalkoxy, NH₂, —NHR¹⁰, —NR¹⁰R¹⁰, NHOR¹⁰, C(O)R¹⁰, C(O)NR¹⁰R¹⁰, C(O)OR¹⁰, C(O)R¹⁰, OC(O)NR¹⁰R¹⁰, NR¹⁰C(O)R¹⁰, NR¹⁰C(O)OR¹⁰, NR¹⁰C(O)NR¹⁰R¹⁰, C(═NR¹⁰)R¹⁰, C(═NR¹⁰)NR¹⁰R¹⁰, NR¹⁰C(═NR¹⁰)NR¹⁰R¹⁰, NR¹⁰S(O)R¹⁰, NR¹⁰S(O)₂R¹⁰, NR¹⁰S(O)₂NR¹⁰R¹⁰, S(O)R¹⁰, S(O)NR¹⁰R¹⁰, S(O)₂R¹⁰, and S(O)₂NR¹⁰R¹⁰, wherein each R¹⁰ is independently selected from H, C₁₋₄ alkyl, C₂₋₄ alkenyl, C₂₋₄ alkynyl, C₁₋₄ alkoxy, C₃₋₁₀ cycloalkyl, C₃₋₁₀ cycloalkyl-C₁₋₄ alkyl-, C₆₋₁₀ aryl, C₆₋₁₀ aryl-C₁₋₄ alkyl, 5-10 membered heteroaryl, 4-10 membered heterocycloalkyl, (5-10 membered heteroaryl)-C₁₋₄ alkyl-, and (4-10 membered heterocycloalkyl)-C₁₋₄ alkyl-, wherein the C₁₋₄ alkyl, C₂₋₄ alkenyl, C₂₋₄ alkynyl, C₁₋₄ alkoxy, C₃₋₁₀ cycloalkyl, C₃₋₁₀ cycloalkyl-C₁₋₄ alkyl-, C₆₋₁₀ aryl, C₆₋₁₀ aryl-C₁₋₄ alkyl-, 5-10 membered heteroaryl, 4-10 membered heterocycloalkyl, (5-10 membered heteroaryl)-C₁₋₄ alkyl-, and (4-10 membered heterocycloalkyl)-C₁₋₄ alkyl- of R^{6a}, R^{6b}, R^{6c} and R¹⁰ are each optionally substituted with 1, 2 or 3 independently selected R^{d} substituents;
or two adjacent R⁷ substituents, taken together with the atoms to which they are attached, form a fused phenyl ring, a fused 4- to 7-membered heterocycloalkyl ring, a fused 5- or 6-membered heteroaryl ring or a fused C₃₋₁₀ cycloalkyl ring, wherein the fused 4- to 7-membered heterocycloalkyl ring and fused 5- or 6-membered heteroaryl ring each have 1-4 heteroatoms as ring members selected from N, O and S and wherein the fused phenyl ring, fused 5- to 7-membered heterocycloalkyl ring, fused 5- or 6-membered heteroaryl ring and fused C₃₋₁₀ cycloalkyl ring are each optionally substituted with 1, 2 or 3 independently selected R^{b} substituents, provided

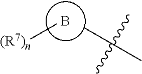

is other than 9-H-carbazol-4-yl or 2,3,4,9-tetrahydro-1H-carbazol-5-yl, each of which is optionally substituted by 1, 2 or 3 independently selected R^{b} substituents;
or two R⁷ substituents attached to the same ring carbon atom of ring B, together with the carbon atom to which they are attached, form a 4- to 7-membered heterocycloalkyl ring having 1-4 heteroatoms as ring members selected from N, O and S or a C₃₋₆ cycloalkyl ring, wherein the 4- to 7-membered heterocycloalkyl ring and C₃₋₆ cycloalkyl ring are each optionally substituted with 1, 2 or 3 independently selected R^{b} substituents;
or R¹ and X² taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1, 2 or 3 independently selected R^{b} substituents;
or R² and X¹ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1, 2 or 3 independently selected R^{b} substituents;
or R² and X³ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1, 2 or 3 independently selected R^{b} substituents;
or R³ and X² taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1, 2 or 3 independently selected R^{b} substituents;
or R³ and X⁴ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1, 2 or 3 independently selected R^{b} substituents;
or R⁴ and X³ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1, 2 or 3 independently selected R^{b} substituents;
each R^{a} is independently selected from H, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{d} substituents;
each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1-3 independently selected R^{f} substituents;
each R^{b} substituent is independently selected from halo, C₁₋₄ alkyl, C₂₋₄ alkenyl, C₂₋₄ alkynyl, C₁₋₄ haloalkyl, C₁₋₄haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₄ alkyl, C₂₋₄ alkenyl, C₂₋₄ alkynyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1-3 independently selected R^{d} substituents;
each R^{c} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{f} substituents independently selected from C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2, 3, 4, or 5 Rⁿ substituents independently selected from C₁₋₄ alkyl, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₁₋₆ haloalkoxy, C₂₋₆ alkenyl, C₂₋₆ alkynyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o}, wherein the C₁₋₄ alkyl, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₁₋₆ haloalkoxy, C₂₋₆ alkenyl, C₂₋₆ alkynyl and C₁₋₄ haloalkyl of Rⁿ are each optionally substituted with 1, 2 or 3 R^{q} substituents;
each R^{g} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1-3 R^{p} substituents independently selected from C₁₋₆ alkyl, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NHR^{r}, NR^{r}C(O)R^{r}, C(O)NR^{r}R^{r}, NR^{r}C(O)OR^{r}, C(═NR)NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NR^{r}S(O)₂R^{r}, NR^{r}S(O)₂NR^{r}R^{r} and S(O)₂NR^{r}R^{r}, wherein the C₁₋₆ alkyl, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} are each optionally substituted with 1, 2 or 3 R^{q} substituents;
or any two R^{a} substituents together with the nitrogen atom to which they are attached form a 4-, 5-, 6-, 7-, 8-, 9- or 10-membered heterocycloalkyl group optionally substituted with 1, 2 or 3 R^{h} substituents independently selected from C₁₋₆ alkyl, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₁₋₆ haloalkyl, C₁₋₆ haloalkoxy, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, S(O)Rⁱ, S(O)NRⁱRⁱ, S(O)₂Rⁱ, NR'S(O)₂Rⁱ, NR'S(O)₂NRⁱRⁱ, and S(O)₂NRⁱRⁱ, wherein the C₁₋₆ alkyl, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, and (4-7 membered heterocycloalkyl)-C₁₋₄ alkyl- of R^{h} are each further optionally substituted by 1, 2, or 3 R^{j} substituents independently selected from C₃₋₆ cycloalkyl, C₆₋₁₀ aryl, 5 or 6-membered heteroaryl, 4-7 membered heterocycloalkyl, C₂₋₄ alkenyl, C₂₋₄ alkynyl, halo, C₁₋₄ alkyl, C₁₋₄ haloalkyl, C₁₋₄haloalkoxy, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR)R^{k}R^{k}, NR^{k}C(═NR^{k})NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, and S(O)₂NR^{k}R^{k}, wherein the C₁₋₄ alkyl, C₃₋₆ cycloalkyl, C₆₋₁₀ aryl, 5- or 6-membered heteroaryl, 4-7 membered heterocycloalkyl, C₂₋₄ alkenyl, C₂₋₆ alkynyl, C₁₋₄ haloalkyl, and C₁₋₄haloalkoxy of R^{j} are each optionally substituted with 1, 2 or 3 independently selected R^{q} substituents; or two R^{h} groups attached to the same carbon atom of the 4- to 10-membered heterocycloalkyl taken together with the carbon atom to which they are attached form a C₃₋₆ cycloalkyl or 4- to 6-membered heterocycloalkyl having 1-2 heteroatoms as ring members selected from O, N or S;
or any two R^{c} substituents together with the nitrogen atom to which they are attached form a 4-, 5-, 6-, or 7-membered heterocycloalkyl group optionally substituted with 1, 2, or 3 independently selected R^{h} substituents;
or any two R^{e} substituents together with the nitrogen atom to which they are attached form a 4-, 5-, 6-, or 7-membered heterocycloalkyl group optionally substituted with 1, 2, or 3 independently selected R^{h} substituents;
or any two R^{g} substituents together with the nitrogen atom to which they are attached form a 4-, 5-, 6-, or 7-membered heterocycloalkyl group optionally substituted with 1, 2, or 3 independently selected R^{h} substituents;
or any two Rⁱ substituents together with the nitrogen atom to which they are attached form a 4-, 5-, 6-, or 7-membered heterocycloalkyl group optionally substituted with 1, 2, or 3 independently selected R^{h} substituents;
or any two R^{k} substituents together with the nitrogen atom to which they are attached form a 4-, 5-, 6-, or 7-membered heterocycloalkyl group optionally substituted with 1, 2, or 3 independently selected R^{h} substituents;
or any two R^{o} substituents together with the nitrogen atom to which they are attached form a 4-, 5-, 6-, or 7-membered heterocycloalkyl group optionally substituted with 1, 2, or 3 independently selected R^{h} substituents; and
each R^{e}, Rⁱ, R^{k}, R^{o} or R^{r} is independently selected from H, C₁₋₄ alkyl, C₃₋₆ cycloalkyl, C₆₋₁₀ aryl, 5 or 6-membered heteroaryl, 4-6 membered heterocycloalkyl, C₁₋₄ haloalkyl, C₂₋₄ alkenyl, and C₂₋₄ alkynyl, wherein the C₁₋₄ alkyl, C₃₋₆ cycloalkyl, C₆₋₁₀ aryl, 5 or 6-membered heteroaryl, 4-6 membered heterocycloalkyl, C₂₋₄ alkenyl, and C₂₋₄ alkynyl of R^{e}, Rⁱ, R^{k}, R^{o} or R^{r} are each optionally substituted with 1, 2 or 3 R^{q} substituents;
each R^{q} is independently selected from OH, CN, —COOH, NH₂, halo, C₁₋₆ haloalkyl, C₁₋₆ alkyl, C₁₋₆ alkoxy, C₁₋₆ alkylthio, phenyl, 5-6 membered heteroaryl, 4-6 membered heterocycloalkyl, C₃₋₆ cycloalkyl, NHR⁸, NR⁸R⁸, and C₁₋₄ haloalkoxy, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5-6 membered heteroaryl and 4-6 membered heterocycloalkyl and each R⁸ is independently C₁₋₆ alkyl;
[symbol] is a single bond or a double bond, wherein ring A includes at least one double bond;
the subscript n is an integer of 1, 2, 3, 4 or 5; and
with the proviso (i) when

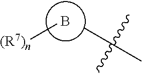

is 2,6-hexahydropyrimidin-1-yl, 2-oxopyrrolidin-1-yl, benzo[d]thiazol-2-yl, 2-amino-4-methyl-5,6-dihydro-1,3-thiazin-4-yl, 4-methyl-6-[4-(morpholine-4-carbonyl)anilino]-5-oxo-pyrazin-2-yl, 5,7-dimethylbenzo[d]oxazol-2-yl, 6-[4-(morpholine-4-carbonyl)phenyl]-7H-pyrrolo[2,3-d]pyrimidin-4-yl, 8-[4-(morpholine-4-carbonyl)anilino]imidazo[1,2-a]pyrazin-6-yl, oxazolo[4,5-b]pyridine-2-yl, or 1-methyl-2-oxo-1,6-naphthridin-3-yl, ring A in Formula (I′) is not 2-pyridyl or 2-pyridyl optionally substituted with halo, methylcarboxy, 1,2,4-triazol-4-yl, 1-piperidinyl, or cyclopropyl;
(ii) when ring B is thiazolo[5,4-b]pyridin-2-yl or 6,7,8,9-tetrahydro-5H-[1,2,4]triazolo[4,3-a]azepin-3-yl, ring A in Formula (I′) is other than 2-quinolyl;
(iii) when ring B is 1-piperazinyl, ring A in Formula (I′) is not 3-(4-benzyloxyphenyl)pyrazolo[1,5-a]pyrimidin-5-yl;
(iv) when

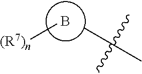

is 6-[1-(dimethylcarbamoyl)-3,6-dihydro-2H-pyridin-4-yl]-7H-pyrrolo[2,3-d]pyrimidin-4-yl, ring A in Formula (I′) is not 1-methylpyrrolo[2,3-b]pyridine-6-yl;
(v) when ring B is 2-oxo-1,2-dihydropyridin-5-yl, 2-oxo-1,2-dihydropyrazin-5-yl or 6-oxo-1H-pyridazin-3-yl, R² is other than t-butyl;
(vi) when

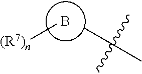

is 3,5-dimethylphenyl, R⁵ is other than 4-amino-1-piperidinyl; and
(vii) the compound is other than 6-((2R,6S)-2,6-dimethylmorpholino)-N-(2-methyl-4′-(trifluoromethoxy)biphenyl-3-yl)pyridazine-3-carboxamide or N-(3-(3-acetyl-2-oxoimidazolidin-1-yl)-2-methylphenyl)-5,6,7,8-tetrahydroquinoline-2-carboxamide.

The present disclosure provides compounds of Formula (I′),
or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
X⁵ is N or CR^{6a};
X⁶ is N or CR^{6b};
Ring B is C₆₋₁₀ aryl, C₃₋₁₀ cycloalkyl, 5- to 14-membered heteroaryl, or 4- to 10-membered heterocycloalkyl, provided ring B is other than 9-H-carbazol-4-yl, 2,3,4,9-tetrahydro-1H-carbazol-5-yl or 1H-tetrazolyl;
R¹, R², R³, R⁴ and R⁷ are each independently selected from H, halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1, 2, 3, or 4 R^{b} substituents;
R⁵ is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR¹¹, SR¹¹, NH₂, —NH—C₁₋₄ alkyl, —N(C₁₋₄ alkyl)₂, NHOR¹¹, C(O)R¹¹, C(O)NR¹¹R¹¹, C(O)OR¹¹, OC(O)R¹¹, OC(O)NR¹¹R¹¹, NR¹¹C(O)R¹¹, NR¹¹C(O)OR¹¹, NR¹¹C(O)NR¹¹R¹¹, C(═NR¹¹)R¹¹, C(═NR¹¹)NR¹¹R¹¹, NR¹¹C(═NR¹¹)NR¹¹R¹¹, NR¹¹S(O)R¹¹, NR¹¹S(O)₂R¹¹, NR¹¹S(O)₂NR¹¹R¹¹, S(O)R¹¹, S(O)NR¹¹R¹¹, S(O)₂R¹¹, and S(O)₂NR¹¹R¹¹, wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl- and (4-10 membered heterocycloalkyl)-C₁₋₄ alkyl- of R⁵ are each optionally substituted with 1, 2 or 3 R^{b} substituents; wherein R⁵ is other than F when ring B is C₃₋₁₀ cycloalkyl, 5- to 14-membered heteroaryl or 4- to 10-membered heterocycloalkyl;
each R¹¹ is independently selected from H, CN, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R¹¹ are each optionally substituted with 1, 2 or 3 R^{b} substituents;
R^{6a}, R^{6b} and R^{6c} are each independently selected from H, C₁₋₄ alkyl, C₃₋₆ cycloalkyl, C₂₋₄ alkenyl, C₂₋₄ alkynyl, halo, CN, OH, C₁₋₄ alkoxy, C₁₋₄ haloalkyl, C₁₋₄ haloalkoxy, NH₂, —NH—C₁₋₄ alkyl, —N(C₁₋₄ alkyl)₂, NHOR¹⁰, C(O)R¹⁰, C(O)NR¹⁰R¹⁰, C(O)OR¹⁰, OC(O)R¹⁰, OC(O)NR¹⁰R¹⁰, NR¹⁰C(O)R¹⁰, NR¹⁰C(O)OR¹⁰, NR¹⁰C(O)NR¹⁰R¹⁰, C(═NR¹⁰)R¹⁰, C(═NR¹⁰)NR¹⁰R¹⁰, NR¹⁰C(═NR¹⁰)NR¹⁰R¹⁰, NR¹⁰S(O)R¹⁰, NR¹⁰S(O)₂R¹⁰, NR¹⁰S(O)₂NR¹⁰R¹⁰, S(O)R¹⁰, S(O)NR¹⁰R¹⁰, S(O)₂R¹⁰, and S(O)₂NR¹⁰R¹⁰, wherein each R¹⁰ is independently H or C₁₋₄ alkyl optionally substituted with 1 or 2 groups independently selected from halo, OH, CN and C₁₋₄ alkoxy, and wherein the C₁₋₄ alkyl, C₃₋₆ cycloalkyl, C₂₋₄ alkenyl and C₂₋₄ alkynyl of R^{6a}, R^{6b}, and R^{6c} are each optionally substituted with 1 or 2 substituents independently selected from halo, OH, CN, C₁₋₄ alkyl and C₁₋₄ alkoxy;
or two adjacent R⁷ substituents, taken together with the atoms to which they are attached, form a fused phenyl ring, a fused 4- to 7-membered heterocycloalkyl ring, a fused 5- or 6-membered heteroaryl ring or a fused C₃₋₁₀ cycloalkyl ring, wherein the fused 4- to 7-membered heterocycloalkyl ring and fused 5- or 6-membered heteroaryl ring each have 1-4 heteroatoms as ring members selected from N, O and S and wherein the fused phenyl ring, fused 5- to 7-membered heterocycloalkyl ring, fused 5- or 6-membered heteroaryl ring and fused C₃₋₁₀ cycloalkyl ring are each optionally substituted with 1 or 2 independently selected R^{b} substituents, provided

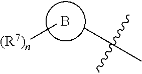

is other than 9-H-carbazol-4-yl or 2,3,4,9-tetrahydro-1H-carbazol-5-yl, each of which is optionally substituted by 1 or 2 independently selected R^{b} substituents;
or two R⁷ substituents attached to the same ring carbon atom of ring B, together with the carbon atom to which they are attached, form a 4- to 7-membered heterocycloalkyl ring having 1-4 heteroatoms as ring members selected from N, O and S or a C₃₋₆ cycloalkyl ring, wherein the 4- to 7-membered heterocycloalkyl ring and C₃₋₆ cycloalkyl ring are each optionally substituted with 1 or 2 independently selected R^{b} substituents;
or R¹ and X² taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R² and X¹ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R² and X³ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R³ and X² taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R³ and X⁴ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R⁴ and X³ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
each R^{a} is independently selected from H, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{d} substituents;
each R^{d} is independently selected from C₁₋₄ alkyl, C₁₋₄haloalkyl, halo, C₃₋₁₀ cycloalkyl, 4-10 membered heterocycloalkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₄ alkyl, C₃₋₁₀ cycloalkyl and 4-10 membered heterocycloalkyl of R^{d} are each further optionally substituted with 1-3 independently selected R^{f} substituents;
each R^{b} substituent is independently selected from halo, C₁₋₄ alkyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═R^{c})NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₄ alkyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1-3 independently selected R^{d} substituents;
each R is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{f} substituents independently selected from C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2, 3, 4, or 5 Rⁿ substituents independently selected from C₁₋₄ alkyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{g} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1-3 independently selected R^{p} substituents;
or any two R^{a} substituents together with the nitrogen atom to which they are attached form a 4-, 5-, 6-, 7-, 8-, 9- or 10-membered heterocycloalkyl group optionally substituted with 1, 2 or 3 R^{h} substituents independently selected from C₁₋₆ alkyl, C₃₋₁₀ cycloalkyl, 4-7 membered heterocycloalkyl, C₆₋₁₀ aryl, 5-6 membered heteroaryl, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₁₋₆ haloalkyl, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, S(O)Rⁱ, S(O)NRⁱRⁱ, S(O)₂Rⁱ, NR'S(O)₂Rⁱ, NR'S(O)₂NRⁱRⁱ, and S(O)₂NRⁱRⁱ, wherein the C₁₋₆ alkyl, C₃₋₁₀ cycloalkyl, 4-7 membered heterocycloalkyl, C₆₋₁₀ aryl, 5-6 membered heteroaryl, C₃₋₁₀ cycloalkyl-C₁₋₄ alkyl-, (5-6 membered heteroaryl)-C₁₋₄ alkyl-, and (4-7 membered heterocycloalkyl)-C₁₋₄ alkyl- of R^{h} are each further optionally substituted by 1, 2, or 3 R^{j} substituents independently selected from C₃₋₆ cycloalkyl, C₆₋₁₀ aryl, 5 or 6-membered heteroaryl, C₂₋₄ alkenyl, C₂₋₄ alkynyl, halo, C₁₋₄ alkyl, C₁₋₄ haloalkyl, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR^{k}C(O)OR^{k}, C(═NR)NR^{k}R^{k}, NR^{k}C(═NR)NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, and S(O)₂NR^{k}R^{k}; or two R^{h} groups attached to the same carbon atom of the 4- to 10-membered heterocycloalkyl taken together with the carbon atom to which they are attached form a C₃₋₆ cycloalkyl or 4- to 6-membered heterocycloalkyl having 1-2 heteroatoms as ring members selected from O, N or S;
or any two R^{c} substituents together with the nitrogen atom to which they are attached form a 4-, 5-, 6-, or 7-membered heterocycloalkyl group optionally substituted with 1, 2, or 3 independently selected R^{h} substituents;
or any two R^{e} substituents together with the nitrogen atom to which they are attached form a 4-, 5-, 6-, or 7-membered heterocycloalkyl group optionally substituted with 1, 2, or 3 independently selected R^{h} substituents;
or any two R^{g} substituents together with the nitrogen atom to which they are attached form a 4-, 5-, 6-, or 7-membered heterocycloalkyl group optionally substituted with 1, 2, or 3 independently selected R^{h} substituents;
or any two Rⁱ substituents together with the nitrogen atom to which they are attached form a 4-, 5-, 6-, or 7-membered heterocycloalkyl group optionally substituted with 1, 2, or 3 independently selected R^{h} substituents;
or any two R^{k} substituents together with the nitrogen atom to which they are attached form a 4-, 5-, 6-, or 7-membered heterocycloalkyl group optionally substituted with 1, 2, or 3 independently selected R^{h} substituents;
or any two R^{o} substituents together with the nitrogen atom to which they are attached form a 4-, 5-, 6-, or 7-membered heterocycloalkyl group optionally substituted with 1, 2, or 3 independently selected R^{h} substituents; and
each R^{e}, Rⁱ, R^{k}, R^{o} or R^{p} is independently selected from H, C₁₋₄ alkyl, C₃₋₆ cycloalkyl, C₆₋₁₀ aryl, 5 or 6-membered heteroaryl, C₁₋₄ haloalkyl, C₂₋₄ alkenyl, and C₂₋₄ alkynyl, wherein the C₁₋₄ alkyl, C₃₋₆ cycloalkyl, C₆₋₁₀ aryl, 5 or 6-membered heteroaryl, C₂₋₄ alkenyl, and C₂₋₄ alkynyl of R^{e}, Rⁱ, R^{k}, R^{o} or R^{p} are each optionally substituted with 1, 2 or 3 R^{q} substituents;
each R^{q} is independently selected from OH, CN, —COOH, NH₂, halo, C₁₋₆ haloalkyl, C₁₋₆ alkyl, C₁₋₆ alkoxy, C₁₋₆ alkylthio, phenyl, 5-6 membered heteroaryl, 4-6 membered heterocycloalkyl, C₃₋₆ cycloalkyl, NHR⁸, NR⁸R⁸, and C₁₋₄ haloalkoxy, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkoxy, C₁₋₄ haloalkyl, C₁₋₄ haloalkoxy, phenyl, C₃₋₁₀ cycloalkyl and 4-6 membered heterocycloalkyl and each R⁸ is independently C₁₋₆ alkyl;
[symbol] is a single bond or a double bond, wherein ring A includes at least one double bond; the subscript n is an integer of 1, 2, 3, 4 or 5; and
with the proviso (i) when

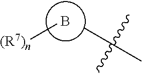

is 2,6-dioxohexahydropyrimidin-1-yl, 2-oxopyrrolidin-1-yl, benzo[d]thiazol-2-yl, 2-amino-4-methyl-5,6-dihydro-1,3-thiazin-4-yl, 4-methyl-6-[4-(morpholine-4-carbonyl)anilino]-5-oxo-pyrazin-2-yl, 5,7-dimethylbenzo[d]oxazol-2-yl, 6-[4-(morpholine-4-carbonyl)phenyl]-7H-pyrrolo[2,3-d]pyrimidin-4-yl, 8-[4-(morpholine-4-carbonyl)anilino]imidazo[1,2-a]pyrazin-6-yl, oxazolo[4,5-b]pyridine-2-yl, or 1-methyl-2-oxo-1,6-naphthridin-3-yl, ring A in Formula (I′) is not 2-pyridyl or 2-pyridyl optionally substituted with halo, methylcarboxy, 1,2,4-triazol-4-yl, 1-piperidinyl, or cyclopropyl;
(ii) when ring B is thiazolo[5,4-b]pyridin-2-yl or 6,7,8,9-tetrahydro-5H-[1,2,4]triazolo[4,3-a]azepin-3-yl, ring A in Formula (I′) is other than 2-quinolyl;
(iii) when ring B is 1-piperazinyl, ring A in Formula (I′) is not 3-(4-benzyloxyphenyl)pyrazolo[1,5-a]pyrimidin-5-yl;
(iv) when

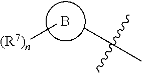

is 6-[1-(dimethylcarbamoyl)-3,6-dihydro-2H-pyridin-4-yl]-7H-pyrrolo[2,3-d]pyrimidin-4-yl, ring A in Formula (I′) is not 1-methylpyrrolo[2,3-b]pyridine-6-yl;
(v) when ring B is 2-oxo-1,2-dihydropyridin-5-yl, 2-oxo-1,2-dihydropyrazin-5-yl or 6-oxo-1H-pyridazin-3-yl, R² is other than t-butyl;
(vi) when

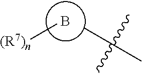

is 3,5-dimethylphenyl, R⁵ is other than 4-amino-1-piperidinyl; and
(vii) the compound is other than 6-((2R,6S)-2,6-dimethylmorpholino)-N-(2-methyl-4′-(trifluoromethoxy)biphenyl-3-yl)pyridazine-3-carboxamide or N-(3-(3-acetyl-2-oxoimidazolidin-1-yl)-2-methylphenyl)-5,6,7,8-tetrahydroquinoline-2-carboxamide.

In some embodiments, when Cy is 2-oxo-1,2-dihydropyridin-5-yl, 2-oxo-1, 2-dihydropyrazin-5-yl or 6-oxo-1,5-dihydropyridazin-3-yl, each of which is optionally substituted, R² is other than C₁₋₆ alkyl.

In some embodiments of compounds of Formula (I′), R⁵ is C₁₋₄ alkyl, halo, CN, OH, cyclopropyl, C₂₋₄ alkynyl, C₁₋₄ alkoxy, C₁₋₄ haloalkyl, C₁₋₄ haloalkoxy, NH₂, —NH—C₁₋₄ alkyl, —N(C₁₋₄ alkyl)₂, NHOR¹¹, C(O)R¹¹, C(O)NR¹¹R¹¹, C(O)OR¹¹, OC(O)R¹¹, OC(O)NR¹¹R¹¹, NR¹¹C(O)R¹¹, NR¹¹C(O)OR¹¹, NR¹¹C(O)NR¹¹R¹¹, C(═NR¹¹)R¹¹, C(═NR¹¹)NR¹¹R¹¹, NR¹¹C(═NR¹¹)NR¹¹R¹¹, NR¹¹S(O)R¹¹, NR¹¹S(O)₂R¹¹, NR¹¹S(O)₂NR¹¹R¹¹, S(O)R¹¹, S(O)NR¹¹R¹¹, S(O)₂R¹¹, and S(O)₂NR¹¹R¹¹, wherein C₁₋₄ alkyl, cyclopropyl, C₂₋₄ alkynyl and C₁₋₄ alkoxy of R⁵ are each optionally substituted with 1 or 2 halo, OH, CN or OCH₃ substituent and each R¹¹ is independently selected from H and C₁₋₄ alkyl optionally substituted with 1 or 2 halo, OH, CN or OCH₃; wherein R⁵ is other than F when ring B is C₃₋₁₀ cycloalkyl, 5- to 14-membered heteroaryl, or 4- to 10-membered heterocycloalkyl. In some embodiments of compounds of Formula (I′), ring A is aromatic.

In some embodiments of compounds of Formula (I′), ring B is C₆₋₁₀ aryl. In certain embodiments, ring B is phenyl or naphthyl. In certain embodiments, ring B is phenyl. R⁷ is as defined in any embodiment of compounds of Formula (I′) or Formula (I) as described herein. In certain embodiments, each R⁷ is H.

In some embodiments, ring B is 1-cyclohexenyl, cyclohexyl, 3,4-dihydro-2H-pyridin-5-yl, 1,2,3,4-tetrahydropyridin-5-yl, 1-piperidinyl, 2,3-dihydro-1,4-benzodioxin-6-yl, 3,4-dihydro-2H-pyran-5-yl, 4-methyl-3,4-dihydro-2H-1,4-benzoxazin-7-yl, 3,4-dihydro-2H-1,4-benzoxazin-7-yl, 2,3-dihydro-1-benzofuran-6-yl, 2-methyl-2H-indazol-6-yl, 2H-indazol-6-yl, 1-methyl-1H-indazol-4-yl or 1H-indazol-4-yl. R⁷ is as defined in any embodiment of compounds of Formula (I′) or Formula (I) as described herein. In some embodiments, R⁷ is t-butoxycarbonyl or C₁₋₆ alkyl. In certain embodiments,

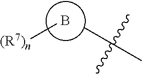

is 1-cyclohexenyl, cyclohexyl, 1-t-butoxycarbonyl-3,4-dihydro-2H-pyridin-5-yl, 1,2,3,4-tetrahydropyridin-5-yl, 1-piperidinyl, 2,3-dihydro-1,4-benzodioxin-6-yl, 3,4-dihydro-2H-pyran-5-yl, 4-methyl-3,4-dihydro-2H-1,4-benzoxazin-7-yl, 3,4-dihydro-2H-1,4-benzoxazin-7-yl, 2,3-dihydro-1-benzofuran-6-yl, 2-methyl-2H-indazol-6-yl, 2H-indazol-6-yl, 1-methyl-1H-indazol-4-yl or 1H-indazol-4-yl.

In some embodiments of compounds of Formula (I′), ring B is C₃₋₁₀ cycloalkyl. In certain embodiments, ring B is cyclopropyl, cyclobutyl, cyclopentyl, cyclohexyl, cyclohexenyl, cycloheptyl or cyclooctyl. R⁷ is as defined in any embodiment of compounds of Formula (I′) or Formula (I) as described herein. In certain embodiments, R⁷ is H.

In some embodiments of compounds of Formula (I′), ring B is 5- to 14-membered heteroaryl. In certain embodiments, ring B is pyridy, primidinyl, pyrazinyl, pyridazinyl, triazinyl, pyrrolyl, pyrazolyl, azolyl, oxazolyl, thiazolyl, imidazolyl, furanyl, thiophenyl, quinolinyl, isoquinolinyl, naphthyridinyl, indolyl, benzothiophenyl, benzofuranyl, benzisoxazolyl, imidazo[1,2-b]thiazolyl, purinyl, thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl. In certain embodiments, ring B is 2-methyl-2H-indazol-6-yl, 1-methyl-1H-indazol-4-yl, 2,3-dihydro-1-benzofuran-6-yl, 4-methyl-3,4-dihydro-2H-1,4-benzoxazin-7-yl or 2,3-dihydro-1,4-benzodioxin-6-yl. R⁷ is as defined in any embodiment of compounds of Formula (I′) or Formula (I) as described herein.

In some embodiments of compounds of Formula (I′), ring B is 4- to 10-membered heterocycloalkyl. In certain embodiments, ring B is azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, 2,3-dihydro-1,4-benzodioxin-6-yl, or thiomorpholino.

In some embodiments, ring B is 3,4-dihydro-2H-pyran-5-yl, 1-piperidinyl or 1,2,3,4-tetrahydropyridin-5-yl. R⁷ is as defined in any embodiment of compounds of Formula (I′) or Formula (I) as described herein.

In some embodiments of compounds of Formula (I′), ring B is phenyl, 5- or 6-membered heteroaryl, C₃₋₆ cycloalkyl or 5- or 6-membered heterocycloalkyl. In certain instances, ring B is phenyl, 2-thiophenyl, 3-thiophenyl, 2-pyridyl, 3-pyridyl, 4-pyridyl, C₃₋₆ cycloalkyl or 3,6-dihydro-2H-pyran-4-yl. R⁷ is as defined in any embodiment of compounds of Formula (I′) or Formula (I) as described herein.

In some embodiments of compounds of Formula (I′), X⁵ and X⁶ are each CH. In certain embodiments, X⁵ is N and X⁶ is CH. In certain embodiments, X⁵ is CH and X⁶ is N. In certain embodiments, X⁵ and X⁶ are each N.

In some embodiments of compounds of Formula (I′), when

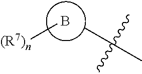

is

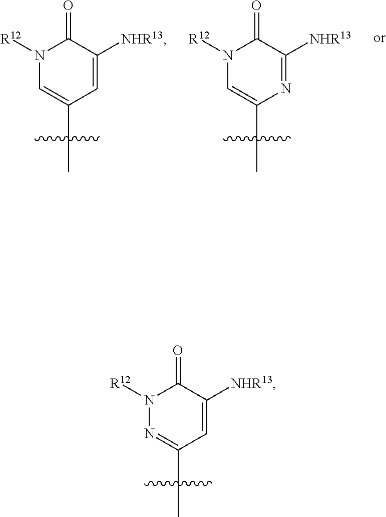

R² is other than t-butyl or C₁₋₆ alkyl, wherein each R¹² is independently H or CH₃ and each R¹³ is independently phenyl substituted with amino and morpholino, phenyl substituted with a group selected from R¹⁴, —C(O)R¹⁴ and (2-amino-2-oxo-ethyl)methylcarbamoyl, 2-pyridyl substituted with 4-methylpiperazin-1-yl or 1-methylpyrrolidin-2-yl, 3-pyridizinyl substituted with morpholino or 4-methylpiperazin-1-yl, 5-methyl-6,7-dihydro-4H-pyrazolo[1,5-a]pyrazine-2-yl, 5-trideuteriomethyl-6,7-dihydro-4H-pyrazolo[1,5-a]pyrazine-2-yl, 5-(2,2-difluoroethyl)-6,7-dihydro-4H-pyrazolo[1,5-a]pyrazine-2-yl, 1-methylpyrazol-3-yl, 4-pyrimidinyl, wherein R¹⁴ is selected from morpholino, 4-ethylpiperazin-1-yl, 1,1-dioxo-1,4-thiazinan-4-yl, 4-oxo-1-piperidinyl, 4-methyl-1-piperidinyl, 4-oxo-piperidin-1-yl, 2-(hydroxymethyl)morpholin-4-yl.

In some embodiments, the present disclosure provides compounds of Formula (I):

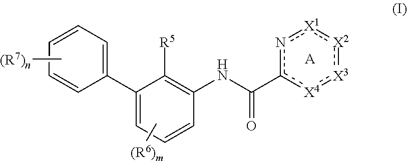

or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
R¹, R², R³, R⁴ and R⁷ are each independently selected from H, halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1, 2, 3, or 4 R^{b} substituents;
R⁵ is C₁₋₄ alkyl, halo, CN, OH, cyclopropyl, C₂₋₄ alkynyl, C₁₋₄ alkoxy, C₁₋₄haloalkyl, C₁₋₄haloalkoxy, NH₂, —NH—C₁₋₄ alkyl, —N(C₁₋₄alkyl)₂, NHOR¹¹, C(O)R¹¹, C(O)NR¹¹R¹¹, C(O)OR¹¹, OC(O)R¹¹, OC(O)NR¹¹R¹¹, NR¹¹C(O)R¹¹, NR¹¹C(O)OR¹¹, NR¹¹C(O)NR¹¹R¹¹, C(═NR¹¹)R¹¹, C(═NR¹¹)NR¹¹R¹¹, NR¹¹C(═NR¹¹)NR¹¹R¹¹, NR¹¹S(O)R¹¹, NR¹¹S(O)₂R¹¹, NR¹¹S(O)₂NR¹¹R¹¹, S(O)R¹¹, S(O)NR¹¹R¹¹, S(O)₂R¹¹, and S(O)₂NR¹¹R¹¹, wherein each R¹¹ is independently selected from H and C₁₋₄ alkyl optionally substituted with 1 or 2 halo, OH, CN or OCH₃;
each R⁶ is independently selected from H, C₁₋₄ alkyl, C₃₋₆ cycloalkyl, C₂₋₄ alkenyl, C₂₋₄ alkynyl, halo, CN, OH, C₁₋₄ alkoxy, C₁₋₄ haloalkyl, C₁₋₄haloalkoxy, NH₂, —NH—C₁₋₄ alkyl, —N(C₁₋₄ alkyl)₂, NHOR¹⁰, C(O)R¹⁰, C(O)NR¹⁰R¹⁰, C(O)OR¹⁰, OC(O)R¹⁰, OC(O)NR¹⁰R¹⁰, NR¹⁰C(O)R¹⁰, NR¹⁰C(O)OR¹⁰, NR¹⁰C(O)NR¹⁰R¹⁰, C(═NR¹⁰)R¹⁰, C(═NR¹⁰)NR¹⁰R¹⁰, NR¹⁰C(═NR¹⁰)NR¹⁰R¹⁰, NR¹⁰S(O)R¹⁰, NR¹⁰S(O)₂R¹⁰, NR¹⁰S(O)₂NR‘OR’°, S(O)R¹⁰, S(O)NR¹⁰R¹⁰, S(O)₂R¹⁰, and S(O)₂NR¹⁰R¹⁰, wherein each R¹⁰ is independently H or C₁₋₄ alkyl optionally substituted with 1 or 2 groups independently selected from halo, OH, CN and C₁₋₄ alkoxy, and wherein the C₁₋₄ alkyl, C₃₋₄ cycloalkyl, C₂₋₄ alkenyl and C₂₋₄ alkynyl of R⁶ are each optionally substituted with 1 or 2 substituents independently selected from halo, OH, CN, C₁₋₄ alkyl and C₁₋₄ alkoxy;
or two adjacent R⁷ substituents, taken together with the carbon atoms to which they are attached, form a fused phenyl ring, a fused 5- to 7-membered heterocycloalkyl ring, a fused 5- or 6-membered heteroaryl ring or a fused C₅₋₆ cycloalkyl ring, wherein the fused 5- to 7-membered heterocycloalkyl ring and fused 5- or 6-membered heteroaryl ring each have 1-4 heteroatoms as ring members selected from N, O and S and wherein the fused phenyl ring, fused 5- to 7-membered heterocycloalkyl ring, fused 5- or 6-membered heteroaryl ring and fused C₃₋₁₀ cycloalkyl ring are each optionally substituted with 1 or 2 independently selected R^{q} substituents;
or R¹ and X² taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R² and X¹ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R² and X³ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R³ and X² taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R³ and X⁴ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
or R⁴ and X³ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents;
each R^{a} is independently selected from H, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{d} substituents;
each R^{d} is independently selected from C₁₋₄ alkyl, C₁₋₄ haloalkyl, halo, C₆₋₁₀ aryl, C₃₋₁₀ cycloalkyl, 5-10 membered heteroaryl, 4-10 membered heterocycloalkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═R^{e})NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₄ alkyl, C₃₋₁₀ cycloalkyl and 4-10 membered heterocycloalkyl of R^{d} are each further optionally substituted with 1-3 independently selected R^{q} substituents or 1-3 independently selected R^{f} substituents;
each R^{b} substituent is independently selected from halo, C₁₋₄ alkyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═R^{c})NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NRS(O)₂NR^{c}R^{c}, S(O)R^{c}, S(O)NR^{c}R^{c}, S(O)₂R^{c} and S(O)₂NR^{c}R^{c}; wherein the C₁₋₄ alkyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1-3 independently selected R^{d} substituents;
each R^{c} is independently selected from H, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{f} substituents independently selected from C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2, 3, 4, or 5 Rⁿ substituents independently selected from C₁₋₄ alkyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{g} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1-3 independently selected R^{p} substituents;
or any two R^{a} substituents together with the nitrogen atom to which they are attached form a 4-, 5-, 6-, 7-, 8-, 9- or 10-membered heterocycloalkyl group optionally substituted with 1, 2 or 3 R^{h} substituents independently selected from C₁₋₆ alkyl, C₃₋₁₀ cycloalkyl, 4-7 membered heterocycloalkyl, C₆₋₁₀ aryl, 5-6 membered heteroaryl, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₁₋₆ haloalkyl, C₂₋₆ alkenyl, C₂₋₆ alkynyl, halo, CN, ORⁱ, SRⁱ, NHORⁱ, C(O)Rⁱ, C(O)NRⁱRⁱ, C(O)ORⁱ, OC(O)Rⁱ, OC(O)NRⁱRⁱ, NHRⁱ, NRⁱRⁱ, NRⁱC(O)Rⁱ, NRⁱC(O)NRⁱRⁱ, NRⁱC(O)ORⁱ, C(═NRⁱ)NRⁱRⁱ, NRⁱC(═NRⁱ)NRⁱRⁱ, S(O)Rⁱ, S(O)NRⁱRⁱ, S(O)₂Rⁱ, NR'S(O)₂Rⁱ, NR'S(O)₂NRⁱRⁱ, and S(O)₂NRⁱRⁱ, wherein the C₁₋₆ alkyl, C₃₋₁₀ cycloalkyl, 4-7 membered heterocycloalkyl, C₆₋₁₀ aryl, 5-6 membered heteroaryl, C₃₋₁₀ cycloalkyl-C₁₋₄ alkyl-, (5-6 membered heteroaryl)-C₁₋₄ alkyl-, and (4-7 membered heterocycloalkyl)-C₁₋₄ alkyl- of R^{h} are each further optionally substituted by 1, 2, or 3 R^{j} substituents independently selected from C₃₋₆ cycloalkyl, C₆₋₁₀ aryl, 5 or 6-membered heteroaryl, C₂₋₄ alkenyl, C₂₋₄ alkynyl, halo, C₁₋₄ alkyl, C₁₋₄ haloalkyl, CN, NHOR^{k}, OR^{k}, SR^{k}, C(O)R^{k}, C(O)NR^{k}R^{k}, C(O)OR^{k}, OC(O)R^{k}, OC(O)NR^{k}R^{k}, NHR^{k}, NR^{k}R^{k}, NR^{k}C(O)R^{k}, NR^{k}C(O)NR^{k}R^{k}, NR OC(O)OR^{k}, C(═NR)NR^{k}R^{k}, NR^{k}C(═NR)NR^{k}R^{k}, S(O)R^{k}, S(O)NR^{k}R^{k}, S(O)₂R^{k}, NR^{k}S(O)₂R^{k}, NR^{k}S(O)₂NR^{k}R^{k}, and S(O)₂NR^{k}R^{k}; or two R^{h} groups attached to the same carbon atom of the 4- to 10-membered heterocycloalkyl taken together with the carbon atom to which they are attached form a C₃₋₆ cycloalkyl or 4- to 6-membered heterocycloalkyl having 1-2 heteroatoms as ring members selected from O, N or S;
or any two R^{c} substituents together with the nitrogen atom to which they are attached form a 4-, 5-, 6-, or 7-membered heterocycloalkyl group optionally substituted with 1, 2, or 3 independently selected R^{h} substituents;
or any two R^{e} substituents together with the nitrogen atom to which they are attached form a 4-, 5-, 6-, or 7-membered heterocycloalkyl group optionally substituted with 1, 2, or 3 independently selected R^{h} substituents;
or any two R^{g} substituents together with the nitrogen atom to which they are attached form a 4-, 5-, 6-, or 7-membered heterocycloalkyl group optionally substituted with 1, 2, or 3 independently selected R^{h} substituents;
or any two Rⁱ substituents together with the nitrogen atom to which they are attached form a 4-, 5-, 6-, or 7-membered heterocycloalkyl group optionally substituted with 1, 2, or 3 independently selected R^{h} substituents;
or any two R^{k} substituents together with the nitrogen atom to which they are attached form a 4-, 5-, 6-, or 7-membered heterocycloalkyl group optionally substituted with 1, 2, or 3 independently selected R^{h} substituents;
or any two R^{o} substituents together with the nitrogen atom to which they are attached form a 4-, 5-, 6-, or 7-membered heterocycloalkyl group optionally substituted with 1, 2, or 3 independently selected R^{h} substituents; and
each R^{e}, Rⁱ, R^{k}, R^{o} or R^{p} is independently selected from H, C₁₋₄ alkyl, C₃₋₆ cycloalkyl, C₆₋₁₀ aryl, 5 or 6-membered heteroaryl, C₁₋₄ haloalkyl, C₂₋₄ alkenyl, and C₂₋₄ alkynyl, wherein the C₁₋₄ alkyl, C₃₋₆ cycloalkyl, C₆₋₁₀ aryl, 5 or 6-membered heteroaryl, C₂₋₄ alkenyl, and C₂₋₄ alkynyl of R^{e}, Rⁱ, R^{k}, R^{o} or R^{p} are each optionally substituted with 1, 2 or 3 R^{q} substituents;
each R^{q} is independently selected from OH, CN, —COOH, NH₂, halo, C₁₋₄ alkyl, C₁₋₄ alkoxy, C₁₋₄ alkylthio, phenyl, 5-6 membered heteroaryl, C₃₋₆ cycloalkyl, NHR^{B}, NR⁸R⁸, and C₁₋₄ haloalkoxy, wherein the C₁₋₄ alkyl, phenyl and 5-6 membered heteroaryl of R^{q} are each optionally substituted with OH, CN, —COOH, NH₂, C₁₋₄ alkoxy, C₃₋₁₀ cycloalkyl and 4-, 5-, or 6-membered heterocycloalkyl and each R⁸ is independently C₁₋₆ alkyl;
the subscript n is an integer of 1, 2, 3, 4 or 5;
the subscript m is an integer of 1, 2, 3 or 4; and
[symbol] is a single bond or a double bond, wherein ring A includes at least one double bond;
with the proviso that the compound is other than 6-((2R,6S)-2,6-dimethylmorpholino)-N-(2-methyl-4′-(trifluoromethoxy)biphenyl-3-yl)pyridazine-3-carboxamide. In some embodiments of compounds of Formula (I), the subscript m is an integer of 1, 2 or 3. The compounds, or pharmaceutically acceptable salts or stereoisomers thereof, as described herein are useful as inhibitors of the PD-1/PD-L1 protein/protein interaction. For example, compounds or pharmaceutically acceptable salts or stereoisomers thereof as described herein can disrupt the PD-1/PD-L1 protein/protein interaction in the PD-1 pathway.

In some embodiments of compounds of Formula (I), R⁵ is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR¹¹, SR¹¹, NH₂, —NH—C₁₋₄ alkyl, —N(C₁₋₄ alkyl)₂, NHOR¹¹, C(O)R¹¹, C(O)NR¹¹R¹¹, C(O)OR¹¹, OC(O)R¹¹, OC(O)NR¹¹R¹¹, NR¹¹C(O)R¹¹, NR¹¹C(O)OR¹¹, NR¹¹C(O)NR¹¹R¹¹, C(═NR¹¹)R¹¹, C(═NR¹¹)NR¹¹R¹¹, NR¹¹C(═NR¹¹)NR¹¹R¹¹, NR¹¹S(O)R¹¹, NR¹¹S(O)₂R¹¹, NR¹¹S(O)₂NR¹¹R¹¹, S(O)R¹¹, S(O)NR¹¹R¹¹, S(O)₂R¹¹, and S(O)₂NR¹¹R¹¹, wherein the C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl- and (4-10 membered heterocycloalkyl)-C₁₋₄ alkyl- of R⁵ are each optionally substituted with 1, 2 or 3 R^{b} substituents; wherein R⁵ is other than F when Cy is C₃₋₁₀ cycloalkyl, 5- to 14-membered heteroaryl, or 4- to 10-membered heterocycloalkyl;
each R¹¹ is independently selected from H, CN, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R¹¹ are each optionally substituted with 1, 2 or 3 R^{b} substituents.

In some embodiments, ring A is other than pyridazinyl group. In some instances, when X² is CR², X³ is CR³ and X⁴ is CR⁴, X¹ is other than N.

In some embodiments, ring A is aromatic.

In some embodiments, ring A has the formula:

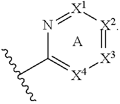

In some embodiments, R¹ and R² taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents.

In some embodiments, R² and R³ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents.

In some embodiments, R³ and R⁴ taken together form a fused phenyl ring, a fused 4- to 10-membered heterocycloalkyl ring, a fused 5- or 6-membered heteroaryl ring or a fused C₃₋₁₀ cycloalkyl ring, wherein the fused 4- to 10-membered heterocycloalkyl ring and fused 5- or 6-membered heteroaryl ring each have 1-4 heteroatoms as ring members selected from N, O and S and wherein the fused phenyl ring, fused 5- or 6-membered heterocycloalkyl ring, fused 5- or 6-membered heteroaryl ring and fused C₅₋₆ cycloalkyl ring are each optionally substituted with 1 or 2 independently selected R^{b} substituents.

In some embodiments, the present disclosure provides compounds having Formula (II):

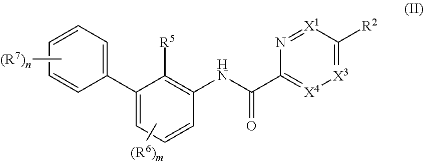

or a pharmaceutically acceptable salt or a stereoisomer thereof. In certain embodiments of compounds of Formula (II), R² is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R² are each optionally substituted with 1, 2, 3, or 4 R^{b} substituents, or a pharmaceutically acceptable salt or a stereoisomer thereof. Other variables of Formula (II) are as defined in Formula (I′), Formula (I) or any embodiment of compounds of Formula (I′) or Formula (I) as described herein. In one embodiment of compounds of Formula (II), R⁵ is CN or C₁₋₄ alkyl optionally substituted with R^{q}. In another embodiment, R⁵ is CH₃ or CN.

In some embodiments, the present disclosure provides compounds having Formula (III):

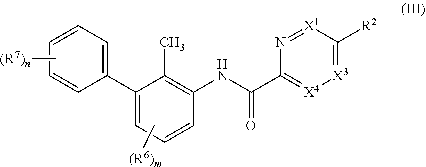

or a pharmaceutically acceptable salt or a stereoisomer thereof. In certain embodiments of compounds of Formula (III), R² is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R² are each optionally substituted with 1, 2, 3, or 4 R^{b} substituents, or a pharmaceutically acceptable salt or a stereoisomer thereof. Other variables of Formula (III) are as defined in Formula (I′), Formula (I) or any embodiment of compounds of Formula (I′) or Formula (I) as described herein.

In some embodiments, the present disclosure provides compounds having Formula (IV):

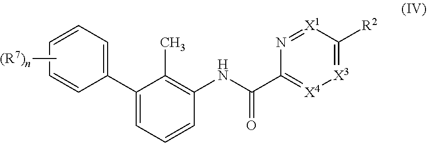

or a pharmaceutically acceptable salt or a stereoisomer thereof, wherein the variables of Formula (IV) are as defined in Formula (I′), Formula (I) or any embodiment of compounds of Formula (I′) or Formula (I) as described herein. In certain embodiments of compounds of Formula (II), R² is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R² are each optionally substituted with 1, 2, 3, or 4 R^{b} substituents, or a pharmaceutically acceptable salt or a stereoisomer thereof.

In some embodiments, the present disclosure provides compounds having Formula (V):

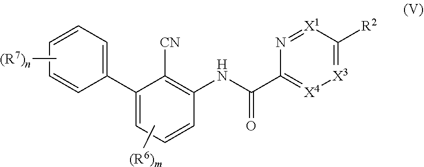

or a pharmaceutically acceptable salt or a stereoisomer thereof, wherein the variables of Formula (V) are as defined in Formula (I′), Formula (I) or any embodiment of compounds of Formula (I′) or Formula (I) as described herein. In certain embodiments of compounds of Formula (V), R² is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R² are each optionally substituted with 1, 2, 3, or 4 R^{b} substituents, or a pharmaceutically acceptable salt or a stereoisomer thereof.

In some embodiments, the present disclosure provides compounds having Formula (VI):

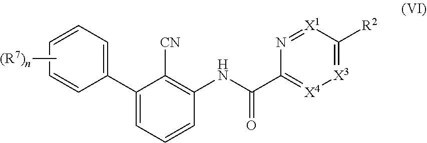

or a pharmaceutically acceptable salt or a stereoisomer thereof, wherein the variables of Formula (VI) are as defined in Formula (I′), Formula (I) or any embodiment of compounds of Formula (I′) or Formula (I) as described herein. In certain embodiments of compounds of Formula (VI), R² is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R² are each optionally substituted with 1, 2, 3, or 4 R^{b} substituents, or a pharmaceutically acceptable salt or a stereoisomer thereof.

In some embodiments, the present disclosure provides compounds having Formula (VII):

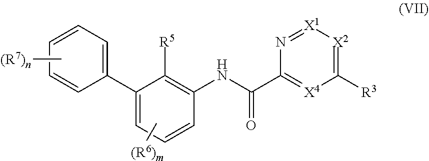

or a pharmaceutically acceptable salt or a stereoisomer thereof, wherein the variables of Formula (VII) are as defined in Formula (I′), Formula (I) or any embodiment of compounds of Formula (I′) or Formula (I) as described herein. In certain embodiments of compounds of Formula (VII), R³ is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R² are each optionally substituted with 1, 2, 3, or 4 R^{b} substituents, or a pharmaceutically acceptable salt or a stereoisomer thereof.

In some embodiments of compounds of Formula I′, I, II, III, IV, V or VI, or a pharmaceutically acceptable salt or a stereoisomer thereof, the moiety

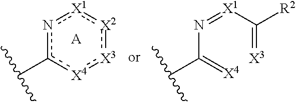

is selected from:

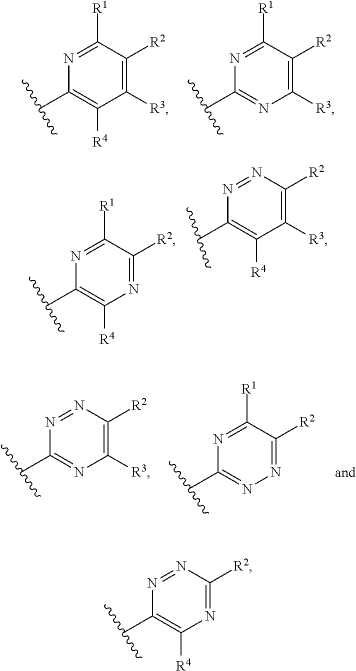

wherein the substituents R¹, R², R³ and R⁴ are as defined in Formula (I′), Formula (I′) or Formula (I) or any embodiment of compounds of Formula (I′) or Formula (I) as described herein. In certain embodiments, at each occurrence, R¹, R³ and R⁴ are each H.

In some embodiments of compounds of Formula I′, I or VII, or a pharmaceutically acceptable salt or a stereoisomer thereof, the moiety the moiety

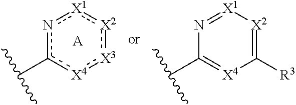

is selected from.

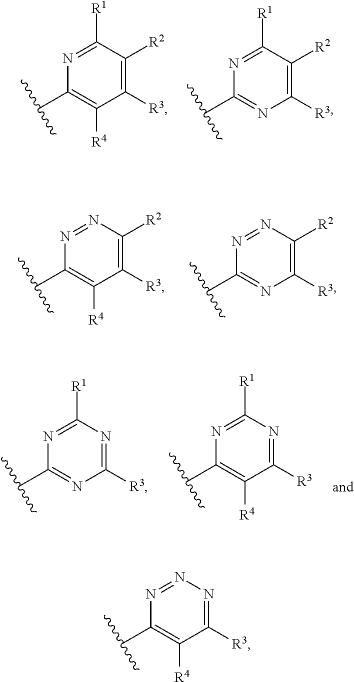

wherein the substituents R¹, R², R³ and R⁴ are as defined in Formula (I′), Formula (I) or any embodiment of compounds of Formula (I′) or Formula (I) as described herein. In certain embodiments, at each occurrence, R¹, R² and R⁴ are each H.

In some embodiments of compounds of Formula I′, I, II, III, IV, V or VI, or a pharmaceutically acceptable salt or a stereoisomer thereof, X¹ is CR¹, X³ is CR³ and X⁴ is CR⁴.

In some embodiments of compounds of Formula I′, I, II, III, IV, V or VI, or a pharmaceutically acceptable salt or a stereoisomer thereof, X¹ is CR¹, X³ is CR³ and X⁴ is N.

In some embodiments of compounds of Formula I′, I, II, III, IV, V or VI, or a pharmaceutically acceptable salt or a stereoisomer thereof, X¹ is CR¹, X³ is N and X⁴ is CR⁴.

In some embodiments of compounds of Formula I′, I, II, III, IV, V or VI, or a pharmaceutically acceptable salt or a stereoisomer thereof, X¹ is N, X³ is CR³ and X⁴ is CR⁴.

In some embodiments of compounds of Formula I′, I or VII, or a pharmaceutically acceptable salt or a stereoisomer thereof, X¹ is CR¹, X² is CR² and X⁴ is CR⁴.

In some embodiments of compounds of Formula I′, I or VII, or a pharmaceutically acceptable salt or a stereoisomer thereof, X¹ is CR¹, X² is CR² and X⁴ is N.

In some embodiments of compounds of Formula I′, I or VII, or a pharmaceutically acceptable salt or a stereoisomer thereof, X¹ is CR¹, X² is N and X⁴ is CR⁴.

In some embodiments of compounds of Formula I′, I or VII, or a pharmaceutically acceptable salt or a stereoisomer thereof, X¹ is N, X² is CR² and X⁴ is CR⁴.

In some embodiments, R¹, R³ and R⁴, are each independently selected from H, C₁₋₆ alkyl, CN, —N(C₁₋₆ alkyl)₂ and halo.

In some embodiments, R¹, R² and R⁴, are each independently selected from H, C₁₋₆ alkyl, CN, —N(C₁₋₆ alkyl)₂ and halo.

In some embodiments, R¹, R², R³, R⁴, R⁶ and R⁷ are each H.

In some embodiments, R¹, R³, R⁴, R⁶ and R⁷ are each H.

In some embodiments, R¹, R², R⁴, R⁶ and R⁷ are each H.

In some embodiments, two adjacent R⁷ substituents on the phenyl ring taken together with the carbon atoms to which they are attached form a 5-, 6- or 7-membered fused heterocycloalkyl optionally substituted by 1 or 2 R^{q} substituents. In certain instances, the fused heterocycloalkyl has carbon and 1 or 2 heteroatoms as ring members selected from O, N or S, wherein the carbon ring atom is optionally oxidized to form carbonyl, the N ring atom is optionally oxidized to form NO and the S ring atom is optionally oxidized to form SO or SO₂.

In some embodiments, the subscript n is 2 and the subscript m is 1.

In some embodiments, R⁵ is C₁₋₄ alkyl or CN.

In some embodiments, R⁵ is CH₃ or CN.

In some embodiments, R⁶ and R⁷ are each H.

In some embodiments of compounds of Formula I′, I, II, III, III, IV, V or VI, R² is C₁₋₄ alkyl substituted with R^{b}. In certain embodiments, R^{b} is NHR^{c} or NR^{c}R^{c}. In certain embodiments, R^{b} is NR^{c}R^{c}. In other embodiments, R^{b} is 2-hydroxyethylamino, 2-hydroxyethyl(methyl)amino, 2-carboxypiperidin-1-yl, (cyanomethyl)amino, (S)-2-carboxypiperidin-1-yl, (R)-2-carboxypiperidin-1-yl or 2-carboxypiperidin-1-yl, each of which is optionally substituted with 1, 2 or 3 R^{q} substituents. In other embodiments, R² is C₁₋₄ alkyl substituted with R^{q}.

In some embodiments of compounds of Formula I′, I, II, III, III, IV, V or VI, R² is C₁₋₄ alkoxy substituted with R^{d}. In certain embodiments, R^{d} is phenyl, 3-cyanophenyl, 3-pyridyl, 2-pyridyl, or 4-pyridyl, each of which is optionally substituted with 1, 2 or 3 R^{q} substituents.

In some embodiments of compounds of Formula I′, I, II, III, III, IV, V or VI, R² is —OCH₂R^{d}. In certain embodiments, R^{d} is phenyl, 3-cyanophenyl, 3-pyridyl, 2-pyridyl, 4-pyridyl, each of which is optionally substituted with 1, 2 or 3 R^{q} substituents.

In some embodiments, when X¹ is N, X³ is CH and X⁴ is CH, R² is other than cis-2,6-dimethylmorpholino. In other embodiments, X¹ is N, X³ is CH and X⁴ is CH, R² is other than 2,6-dimethylmorpholino. In other embodiments, X¹ is N, X³ is CH and X⁴ is CH, R² is other than 6-membered heterocycloalkyl substituted with 1 or 2 C₁₋₆ alkyl, wherein the heterocycloalkyl has N and O as ring members. In other embodiments, ring A is other than 6-(2,6-dimethylmorpholino)pyridazin-3-yl.

In some embodiments of compounds of Formula I′, I or VII, R³ is C₁₋₄ alkyl substituted with R^{b}. In certain embodiments, R^{b} is NHR^{c} or NR^{c}R^{c}. In other embodiments, R^{b} is 2-hydroxyethylamino, 2-hydroxyethyl(methyl)amino, 2-carboxypiperidin-1-yl, (cyanomethyl)amino, (S)-2-carboxypiperidin-1-yl, (R)-2-carboxypiperidin-1-yl or 2-carboxypiperidin-1-yl, each of which is optionally substituted with 1, 2 or 3 R^{q} substituents. In other embodiments, R³ is C₁₋₄ alkyl substituted with R^{q}.

In some embodiments of compounds of Formula I′, I or VII, R³ is C₁₋₄ alkoxy substituted with R^{d}. In certain embodiments, R^{d} is phenyl, 3-cyanophenyl, 3-pyridyl, 2-pyridyl, or 4-pyridyl, each of which is optionally substituted with 1, 2 or 3 R^{q} substituents.

In some embodiments of compounds of Formula I′, I or VII, R³ is —OCH₂R^{d}. In certain embodiments, R^{d} is phenyl, 3-cyanophenyl, 3-pyridyl, 2-pyridyl, 4-pyridyl, each of which is optionally substituted with 1, 2 or 3 R^{q} substituents.

In some embodiments of compounds of Formula I′, I, II, III, IV, V or VI, R³ is 2-hydroxyethylaminomethyl, 2-hydroxyethyl(methyl)aminomethyl, 2-carboxypiperidin-1-ylmethyl, (cyanomethyl)aminomethyl, (S)-2-carboxypiperidin-1-ylmethyl, (R)-2-carboxypiperidin-1-ylmethyl, 2-carboxypiperidin-1-ylmethyl, benzyloxy, 2-cyanobenzyloxy, 3-cyanobenzyloxy, 4-cyanobenzyloxy, 2-pyridylmethoxy, 3-pyridylmethoxy, or 4-pyridylmethoxy, each of which is optionally substituted with 1, 2 or 3 R^{q} substituents. In certain embodiments, R³ is 2-hydroxyethylaminomethyl, 2-carboxypiperidin-1-ylmethyl, (S)-2-carboxypiperidin-1-ylmethyl, (R)-2-carboxypiperidin-1-ylmethyl or (3-cyanobenzyl)oxy, each of which is optionally substituted with 1, 2 or 3 R^{q} substituents.

In some embodiments of compounds of Formula I′, R^{6a}, R^{6b} and R^{6c} are each independently selected from H, halo, CN, N(C₁₋₆ alkyl)₂, C₁₋₆ alkyl and C₁₋₆ alkoxy, wherein the C₁₋₆ alkyl are each optionally substituted with 1 or 2 substituents independently selected from halo, OH, CN, C₁₋₄ alkyl and C₁₋₄ alkoxy.

In some embodiments of compounds of Formula I′, R^{6a}, R^{6b} and R^{6c} are each independently selected from H, halo, CN, N(CH₃)₂ and CH₃.

In some embodiments of compounds of Formula I, II, III, V, or VII, R⁶ is H, halo, CN, N(C₁₋₆ alkyl)₂, C₁₋₆ alkyl or C₁₋₆ alkoxy, wherein the C₁₋₆ alkyl and C₁₋₆ alkoxy are each optionally substituted with 1-3 R^{q} substituents.

In some embodiments of compounds of Formula I, II, III, V or VIII, R⁶ is H, halo, CN, N(CH₃)₂ or CH₃.

In some embodiments, provided herein is a compound of Formula (I′):

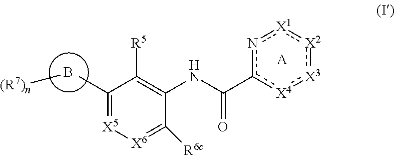

or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
X⁵ is N or CR^{6a};
X⁶ is CR^{6b};
ring B is phenyl, cyclohexyl, piperidinyl, or tetrahydropyridinyl;
R¹, R², R³, R⁴ and R⁷ are each independently selected from H, halo, C₁₋₆ alkyl, C₂₋₆ alkenyl, C₂₋₆ alkynyl, C₁₋₆ haloalkyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, OR^{a}, NHR^{a}, and NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1, 2, 3, or 4 R^{b} substituents;
R⁵ is C₁₋₆ alkyl, C₂₋₆ alkenyl, C₂₋₆ alkynyl, C₁₋₆ haloalkyl, C₁₋₆ haloalkoxy, or CN, wherein the C₁₋₆ alkyl, C₂₋₆ alkenyl, C₂₋₆ alkynyl, C₁₋₆ haloalkyl, and C₁₋₆ haloalkoxy of R⁵ are each optionally substituted with 1, 2 or 3 R^{b} substituents;
R^{6a}, R^{6b} and R^{6c} are each independently selected from H, C₁₋₄ alkyl, C₂₋₄ alkenyl, C₂₋₄ alkynyl, halo, CN, OH, C₁₋₄ alkoxy, C₁₋₄ haloalkyl, NH₂, —NH—C₁₋₄ alkyl, and —N(C₁₋₄ alkyl)₂, wherein the C₁₋₄ alkyl, C₂₋₄ alkenyl and C₂₋₄ alkynyl of R^{6a}, R^{6b}, and R^{6c} are each optionally substituted with 1 or 2 substituents independently selected from halo, OH, CN, C₁₋₄ alkyl and C₁₋₄ alkoxy;
or two adjacent R⁷ substituents, taken together with the atoms to which they are attached, form a fused 4- to 7-membered heterocycloalkyl ring or a fused 5- or 6-membered heteroaryl ring, wherein the fused 4- to 7-membered heterocycloalkyl ring and fused 5- or 6-membered heteroaryl ring each have 1-4 heteroatoms as ring members selected from N and O and wherein the fused 5- to 7-membered heterocycloalkyl ring and fused 5- or 6-membered heteroaryl ring are each optionally substituted with 1 or 2 independently selected R^{b} substituents;
each R^{a} is independently selected from H, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{d} substituents;
each R^{d} is independently selected from C₁₋₄ alkyl, C₁₋₄haloalkyl, halo, C₃₋₁₀ cycloalkyl, 4-10 membered heterocycloalkyl, CN, NH₂, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, and NR^{e}C(O)R^{e}, and S(O)₂NR^{e}R^{e}, wherein the C₁₋₄ alkyl, C₃₋₁₀ cycloalkyl and 4-10 membered heterocycloalkyl of R^{d} are each further optionally substituted with 1-3 independently selected R^{f} substituents;
each R^{b} substituent is independently selected from halo, C₁₋₄ alkyl, C₁₋₄ haloalkyl, C₁₋₄ haloalkoxy, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, NHR^{c}, NR^{c}R^{c}, NR^{c}C(O)R^{c}, and NR^{c}C(O)OR^{c}; wherein the C₁₋₄ alkyl, C₁₋₄ haloalkyl, and C₁₋₄haloalkoxy of R^{b} are each further optionally substituted with 1-3 independently selected R^{d} substituents;
each R is independently selected from H, C₁₋₆ alkyl, C₁₋₄ haloalkyl, C₂₋₆ alkenyl, and C₂₋₆ alkynyl, wherein the C₁₋₆ alkyl, C₂₋₆ alkenyl, and C₂₋₆ alkynyl of R^{c} are each optionally substituted with 1, 2, 3, 4, or 5 R^{f} substituents independently selected from C₁₋₄ alkyl, C₁₋₄ haloalkyl, C₂₋₆ alkenyl, C₂₋₆ alkynyl, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, and NR^{g}C(O)R^{g};
each R^{g} is independently selected from H, C₁₋₆ alkyl, C₁₋₄haloalkyl, C₂₋₆ alkenyl, and C₂₋₆ alkynyl;
each R^{e} is independently selected from H, C₁₋₄ alkyl, C₁₋₄ haloalkyl, C₂₋₄ alkenyl, and C₂₋₄ alkynyl;
[symbol] is a single bond or a double bond, wherein ring A includes at least one double bond; and
the subscript n is an integer of 1, 2, 3, 4 or 5.

In some embodiments, provided herein is a compound of Formula (I′):

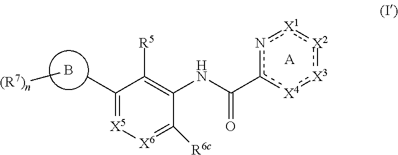

or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
X⁵ is N or CR^{6a};
X⁶ is CR^{6b};
ring B is phenyl, cyclohexyl, piperidinyl, or tetrahydropyridinyl;
R¹, R², R³, R⁴ and R⁷ are each independently selected from H, halo, C₁₋₆ alkyl, C₂₋₆ alkenyl, C₂₋₆ alkynyl, 5-14 membered heteroaryl, 4-10 membered heterocycloalkyl, (5-14 membered heteroaryl)-C₁₋₄ alkyl-, (4-10 membered heterocycloalkyl)-C₁₋₄ alkyl-, CN, OR^{a}, NHR^{a}, and NR^{a}R^{a}, wherein the C₁₋₆ alkyl, C₂₋₆ alkenyl, C₂₋₆ alkynyl, 5-14 membered heteroaryl, 4-10 membered heterocycloalkyl, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1, 2, 3, or 4 R^{b} substituents;
R⁵ is C₁₋₆ alkyl or CN;
R^{6a}, R^{6b} and R^{6c} are each independently selected from H, C₁₋₄ alkyl, C₂₋₄ alkenyl, C₂₋₄ alkynyl, halo, CN, OH, NH₂, —NH—C₁₋₄ alkyl, and —N(C₁₋₄ alkyl)₂;
or two adjacent R⁷ substituents, taken together with the atoms to which they are attached, form a fused 4- to 7-membered heterocycloalkyl ring or a fused 5- or 6-membered heteroaryl ring, wherein the fused 4- to 7-membered heterocycloalkyl ring and fused 5- or 6-membered heteroaryl ring each have 1-4 heteroatoms as ring members selected from N and O and wherein the fused 5- to 7-membered heterocycloalkyl ring and fused 5- or 6-membered heteroaryl ring are each optionally substituted with 1 or 2 independently selected R^{b} substituents;
each R^{a} is independently selected from H, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R are each optionally substituted with 1, 2, 3, 4, or 5 R^{d} substituents;
each R^{d} is independently selected from C₁₋₄ alkyl, C₁₋₄haloalkyl, halo, CN, and NH₂;
each R^{b} substituent is independently selected from halo, C₁₋₄ alkyl, C₁₋₄ haloalkyl, C₁₋₄ haloalkoxy, CN, OH, NH₂, C(O)OR^{c}, NHR^{c}, and NR^{c}R^{c};
each R^{c} is independently selected from H, C₁₋₆ alkyl, C₁₋₄ haloalkyl, C₂₋₆ alkenyl, and C₂₋₆ alkynyl, wherein the C₁₋₆ alkyl, C₂₋₆ alkenyl, and C₂₋₆ alkynyl of R^{c} are each optionally substituted with 1, 2, 3, 4, or 5 R^{f} substituents independently selected from C₁₋₄ alkyl, C₁₋₄ haloalkyl, C₂₋₆ alkenyl, C₂₋₆ alkynyl, halo, CN, OR^{g}, C(O)R^{g}, C(O)NR^{g}R^{g}, C(O)OR^{g}, NHR^{g}, NR^{g}R^{g}, and NR^{g}C(O)R^{g};
each R^{g} is independently selected from H, C₁₋₆ alkyl, C₂₋₆ alkenyl, and C₂₋₆ alkynyl;
[symbol] is a single bond or a double bond, wherein ring A includes at least one double bond; and
the subscript n is an integer of 1, 2, 3, 4 or 5.

In some embodiments, provided herein is a compound of Formula (I′):

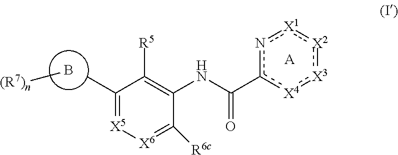

or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
X⁵ is N or CR^{6a};
X⁶ is CR^{6b};
ring B is phenyl, cyclohexyl, piperidinyl, or tetrahydropyridinyl;
R¹, R², R³, R⁴ and R⁷ are each independently selected from H, halo, C₁₋₆ alkyl, C₂₋₆ alkenyl, C₂₋₆ alkynyl, 5-14 membered heteroaryl, 4-10 membered heterocycloalkyl, (5-14 membered heteroaryl)-C₁₋₄ alkyl-, (4-10 membered heterocycloalkyl)-C₁₋₄ alkyl-, CN, OR^{a}, NHR^{a}, and NR^{a}R^{a}, wherein the C₁₋₆ alkyl, C₂₋₆ alkenyl, C₂₋₆ alkynyl, 5-14 membered heteroaryl, 4-10 membered heterocycloalkyl, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1 or 2, R^{b} substituents;
R⁵ is C₁₋₆ alkyl or CN;
R^{6a}, R^{6b} and R^{6c} are each independently selected from H, C₁₋₄ alkyl and halo;
or two adjacent R⁷ substituents, taken together with the atoms to which they are attached, form a fused 4- to 7-membered heterocycloalkyl ring or a fused 5- or 6-membered heteroaryl ring, wherein the fused 4- to 7-membered heterocycloalkyl ring and fused 5- or 6-membered heteroaryl ring each have 1-4 heteroatoms as ring members selected from N and O and wherein the fused 5- to 7-membered heterocycloalkyl ring and fused 5- or 6-membered heteroaryl ring are each optionally substituted with 1 or 2 independently selected R^{b} substituents;
each R^{a} is independently selected from H, CN, C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1 or 2 R^{d} substituents;
each R^{d} is independently selected from C₁₋₄ alkyl, halo, CN, and NH₂;
each R^{b} substituent is independently selected from halo, C₁₋₄ alkyl, CN, OH, NH₂, C(O)OR^{c}, NHR^{c}, and NR^{c}R^{c};
each R is independently selected from H, C₁₋₆ alkyl, C₂₋₆ alkenyl, and C₂₋₆ alkynyl, wherein the C₁₋₆ alkyl, C₂₋₆ alkenyl, and C₂₋₆ alkynyl of R^{c} are each optionally substituted with 1, 2 or R^{f} substituents independently selected from C₁₋₄ alkyl, C₂₋₆ alkenyl, C₂₋₆ alkynyl, halo, CN, and OR^{g};
each R^{g} is independently selected from H and C₁₋₆ alkyl;
[symbol] is a single bond or a double bond, wherein ring A includes at least one double bond; and
the subscript n is an integer of 1, 2, 3, 4 or 5.

It is further appreciated that certain features of the invention, which are, for clarity, described in the context of separate embodiments, can also be provided in combination in a single embodiment (while the embodiments are intended to be combined as if written in multiply dependent form). Conversely, various features of the invention which are, for brevity, described in the context of a single embodiment, can also be provided separately or in any suitable subcombination. Thus, it is contemplated as features described as embodiments of the compounds of Formula (I′) or formula (I) can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ— includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl,” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl,” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl,” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene,” employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl and the like.

The term “alkoxy,” employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “amino,” employed alone or in combination with other terms, refers to a group of formula —NH₂.

The term “carbamyl,” employed alone or in combination with other terms, refers to a group of formula —C(O)NH₂.

The term “carbonyl,” employed alone or in combination with other terms, refers to a —C(═O)— group, which also may be written as C(O).

The term “cyano” or “nitrile,” employed alone or in combination with other terms, refers to a group of formula —C—N, which also may be written as —CN.

The terms “halo” or “halogen,” used alone or in combination with other terms, refers to fluoro, chloro, bromo and iodo. In some embodiments, “halo” refers to a halogen atom selected from F, C₁, or Br. In some embodiments, halo groups are F.

The term “haloalkyl,” employed alone or in combination with other terms, as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1} halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CCl₃, CHCl₂, C₂Cl₅ and the like. In some embodiments, the haloalkyl group is a fluoroalkyl group.

The term “haloalkoxy,” employed alone or in combination with other terms, refers to a group of formula —O-haloalkyl, wherein the haloalkyl group is as defined above. The term “Cₙ₋ₘ haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “sulfido” refers to a sulfur atom as a divalent substituent, forming a thiocarbonyl group (C═S) when attached to carbon.

The term “aromatic” refers to a carbocycle or heterocycle having one or more polyunsaturated rings having aromatic character (i.e., having (4n+2) delocalized π (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, indanyl, indenyl and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl. In some embodiments, the aryl group is naphthyl.

The term “heteroaryl” or “heteroaromatic,” employed alone or in combination with other terms, refers to a monocyclic or polycyclic aromatic heterocycle having at least one heteroatom ring member selected from sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridintl (pyridyl), pyrimidinyl, pyrazinyl, pyridazinyl, pyrrolyl, pyrazolyl, azolyl, oxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, benzothiophenyl, benzofuranyl, benzisoxazolyl, imidazo[1,2-b]thiazolyl, purinyl, and the like.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6 or 7 ring-forming carbons (C₃₋₇). In some embodiments, the cycloalkyl group has 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from nitrogen, sulfur oxygen and phosphorus, and which has 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic (e.g., having two fused or bridged rings) ring systems. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, and thiomorpholino.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone-enol pairs, amide-imidic acid pairs, lactam-lactim pairs, enamine-imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

In some embodiments, the compounds of the invention, or salts thereof, are substantially isolated. By “substantially isolated” is meant that the compound is at least partially or substantially separated from the environment in which it was formed or detected. Partial separation can include, e.g., a composition enriched in the compounds of the invention. Substantial separation can include compositions containing at least about 50%, at least about 60%, at least about 70%, at least about 80%, at least about 90%, at least about 95%, at least about 97%, or at least about 99% by weight of the compounds of the invention, or salt thereof.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### II. Synthesis

Compounds of the invention, including salts thereof, can be prepared using known organic synthesis techniques and can be synthesized according to any of numerous possible synthetic routes, such as those in the Schemes below.

The reactions for preparing compounds of the invention can be carried out in suitable solvents which can be readily selected by one of skill in the art of organic synthesis. Suitable solvents can be substantially non-reactive with the starting materials (reactants), the intermediates or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups is described, e.g., in Kocienski, Protecting Groups, (Thieme, 2007); Robertson, Protecting Group Chemistry, (Oxford University Press, 2000); Smith et al., March's Advanced Organic Chemistry: Reactions, Mechanisms, and Structure, 6^{th} Ed. (Wiley, 2007); Peturssion et al., “Protecting Groups in Carbohydrate Chemistry,” J. Chem. Educ., 1997, 74(11), 1297; and Wuts et al., Protective Groups in Organic Synthesis, 4th Ed., (Wiley, 2006).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry or by chromatographic methods such as high performance liquid chromatography (HPLC) or thin layer chromatography (TLC).

The Schemes below provide general guidance in connection with preparing the compounds of the invention. One skilled in the art would understand that the preparations shown in the Schemes can be modified or optimized using general knowledge of organic chemistry to prepare various compounds of the invention.

Compounds of Formula (I′) and Formula (I) can be synthesized using a process shown in Scheme 1. In Scheme 1, a suitable halo (W₁)-substituted aromatic amine 1 is reacted with a suitable coupling reagent 2 (where M is, e.g., —B(OH)₂) to produce compound 3 under standard metal catalyzed cross-coupling reaction conditions (such as Suzuki coupling reaction, e.g., in the presence of a palladium catalyst (e.g., 1,1′-bis(diphenylphosphino)ferrocene]-dichloropalladium(II)) and a base (e.g., a bicarbonate or a carbonate base)). Then the aromatic amine 3 is reacted with an acid of formula 4 under suitable conditions forming an amide bond to provide the product of formula (I) using coupling reagents such as, but not limited to, HATU and DIPEA.

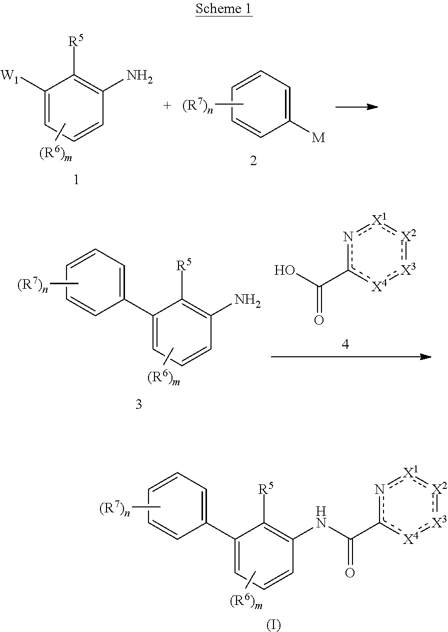

Compounds of the invention (8a) can be synthesized using a process shown in Scheme 2. The aromatic amine 3 is reacted with an acid of formula 5 under suitable conditions forming an amide bond to provide the product 6, using coupling reagents such as, but not limited to, HATU and DIPEA. The compound of formula 7 can be synthesized by coupling the halo group (W₂) of 6 with a vinyl reagent (e.g., vinyl boronic acid pinacol ester) under standard coupling reaction conditions (such as Suzuki coupling reaction, e.g., in the presence of a palladium catalyst (e.g., 1,1′-bis(diphenylphosphino)ferrocene]dichloropalladium(II)) and a base (e.g., a bicarbonate or a carbonate base)). The vinyl group in compound 7 is oxidatively cleaved to afford an aldehyde of formula 8 in the presence of suitable reagents such as, but not limited to, OsO₄ and NaIO₄. Then compounds of formula 8a are obtained by a reductive amination between the compound of formula 8 and a suitable amine in a proper solvent such as THF or DCM using a reducing agent such as, but not limited to, sodium triacetoxyborohydride, optionally in the presence of an acid such as acetic acid.

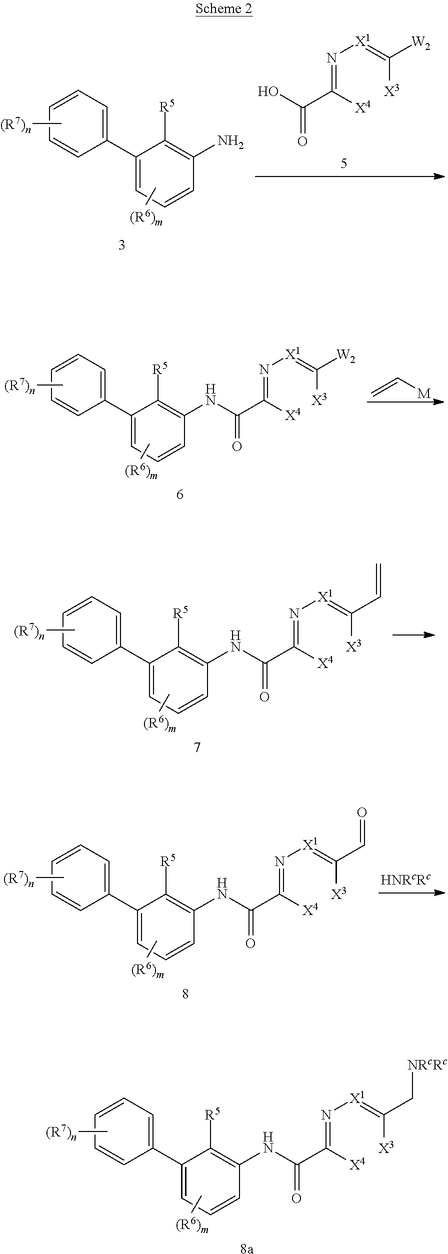

Compounds of formula 8a can be alternatively synthesized using a process shown in Scheme 3. The aromatic amine 3 is reacted with an acid of formula 9 under suitable conditions to form an amide bond providing the product of formula 10, using coupling reagents such as, but not limited to, HATU and DIPEA. Subsequent reduction of the ester group in compound 10 can give an alcohol of formula 11 using a suitable reducing agent such as, but not limited to, lithium aluminum hydride. The alcohol unit in compound 11 was oxidized to aldehyde in compound 8 with a suitable oxidant such as, but not limited to, Dess-Martin periodinane. Then the compound of formula 8a was obtained by a reductive amination between formula 8 and a suitable amine in a proper solvent such as THF or DCM using a reducing agent such as, but not limited to, sodium triacetoxyborohydride, optionally in the presence of an acid such as acetic acid.

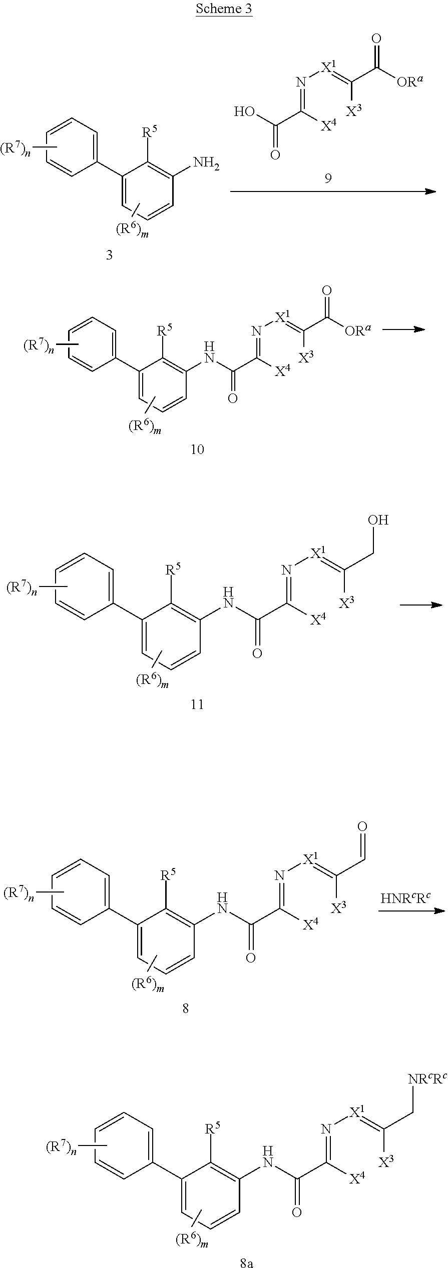

Compounds of the invention (15a) can be synthesized using a process shown in Scheme 4. The aromatic amine 3 is reacted with an acid of formula 12 under suitable conditions forming an amide bond to provide the product of formula 13, using coupling reagents such as, but not limited to, HATU and DIPEA. The compound of formula 14 can be synthesized by coupling the halo group (W₃) of 13 with a vinyl reagent (e.g., vinyl boronic acid pinacol ester) under standard coupling reaction conditions (such as Suzuki coupling reaction, e.g., in the presence of a palladium catalyst (e.g., 1,1′-bis(diphenylphosphino)ferrocene]dichloropalladium(II)) and a base (e.g., a bicarbonate or a carbonate base)). The vinyl group in compound 14 is oxidatively cleaved to afford an aldehyde of formula 15 in the presence of suitable reagents such as, but not limited to, OsO₄ and NaIO₄. Then the compound of formula 15a is obtained by a reductive amination between the compound of formula 15 and a suitable amine in a proper solvent such as THF or DCM using a reducing agent such as, but not limited to, sodium triacetoxyborohydride, optionally in the presence of an acid such as acetic acid.

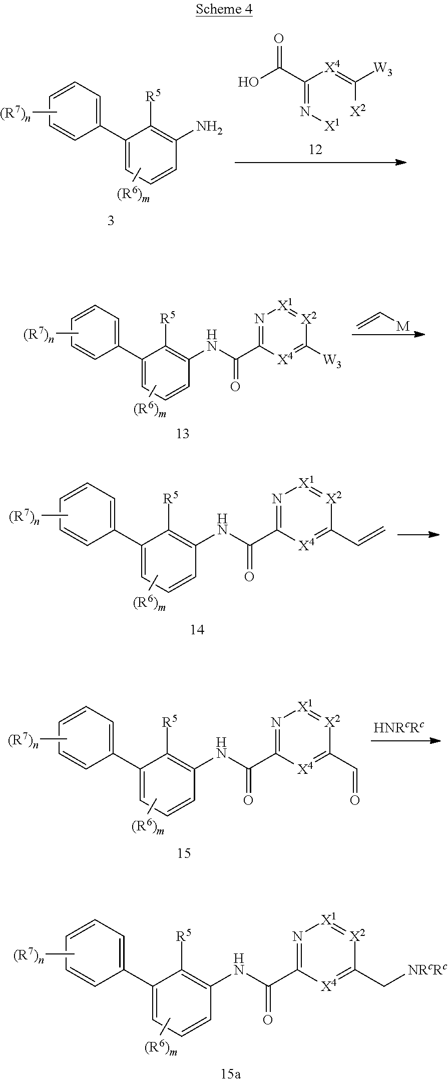

Compounds of Formula 19 can be prepared using procedures as outlined in Scheme 5. Acids of formula 4 can react with aromatic amines of formula 16 under amide coupling conditions [e.g., in the presence of 1-[Bis(dimethylamino)methylene]-1H-1,2,3-triazolo[4,5-b]pyridinium 3-oxid hexafluorophosphate (HATU) and a suitable base such as N,N-Diisopropylethylamine (DIPEA)] to give amide derivatives of formula 17. The halide (e.g., Hal¹=Cl, Br, I) in compounds 17 can be coupled to compounds of formula 18, in which M is a boronic acid, boronic ester or an appropriately substituted metal [e.g., M is B(OR)₂, Sn(Alkyl)₄, or Zn-Hal], under Suzuki coupling conditions (e.g., in the presence of a palladium catalyst and a suitable base) or Stille coupling conditions (e.g., in the presence of a palladium catalyst), or Negishi coupling conditions (e.g., in the presence of a palladium catalyst) to give derivatives of formula 19. Alternatively, compound 18 can be a cyclic amine (where M is H and attached to an amine nitrogen in ring A) and the coupling of aryl halide 17 with the cyclic amine 18 can be performed under Buchwald amination conditions (e.g., in the presence of a palladium catalyst and a base such as sodium tert-butoxide).

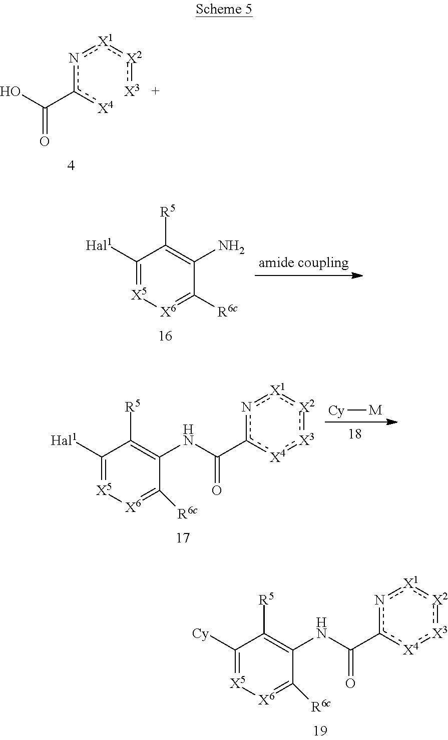

Alternatively, compounds of formula 19 can also be prepared using reaction sequence as outlined in Scheme 6. Coupling of aromatic halides of formula 16 with compounds of formula 18 can be achieved under similar conditions as described in Scheme 5 (e.g., conditions used for coupling of compounds 17 and compounds 18) to give compounds of formula 20. Acids of formula 4 can react with aromatic amines of formula 20 under amide coupling conditions to give compounds of formula 19.

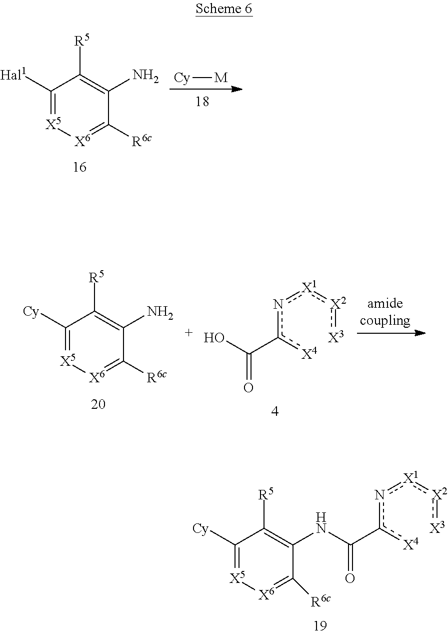

### III. Uses of the Compounds

Compounds of the present disclosure can inhibit the activity of PD-1/PD-L1 protein/protein interaction and, thus, are useful in treating diseases and disorders associated with activity of PD-1 and the diseases and disorders associated with PD-L1 including its interaction with other proteins such as PD-1 and B7-1 (CD80). In certain embodiments, the compounds of the present disclosure, or pharmaceutically acceptable salts or stereoisomers thereof, are useful for therapeutic administration to enhance stimulate and/or increase immunity in cancer or chronic infection, including enhancement of response to vaccination. In some embodiments, the present disclosure provides a method for inhibiting the PD-1/PD-L1 protein/protein interaction. The method includes administering to an individual or a patient a compound of Formula (I′), Formula (I) or of any of the formulas as described herein, or of a compound as recited in any of the claims and described herein, or a pharmaceutically acceptable salt or a stereoisomer thereof. The compounds of the present disclosure can be used alone, in combination with other agents or therapies or as an adjuvant or neoadjuvant for the treatment of diseases or disorders, including cancer or infection diseases. For the uses described herein, any of the compounds of the disclosure, including any of the embodiments thereof, may be used.

The compounds of the present disclosure inhibit the PD-1/PD-L1 protein/protein interaction, resulting in a PD-1 pathway blockade. The blockade of PD-1 can enhance the immune response to cancerous cells and infectious diseases in mammals, including humans. In some embodiments, the present disclosure provides treatment of an individual or a patient in vivo using a compound of Formula (I′), Formula (I) or a salt or stereoisomer thereof such that growth of cancerous tumors is inhibited. A compound of Formula (I′), Formula (I) or of any of the formulas as described herein, or a compound as recited in any of the claims and described herein, or a salt or stereoisomer thereof, can be used to inhibit the growth of cancerous tumors. Alternatively, a compound of Formula (I′), Formula (I) or of any of the formulas as described herein, or a compound as recited in any of the claims and described herein, or a salt or stereoisomer thereof, can be used in conjunction with other agents or standard cancer treatments, as described below. In one embodiment, the present disclosure provides a method for inhibiting growth of tumor cells in vitro. The method includes contacting the tumor cells in vitro with a compound of Formula (I′), Formula (I) or of any of the formulas as described herein, or of a compound as recited in any of the claims and described herein, or of a salt or stereoisomer thereof. In another embodiment, the present disclosure provides a method for inhibiting growth of tumor cells in an individual or a patient. The method includes administering to the individual or patient in need thereof a therapeutically effective amount of a compound of Formula (I′), Formula (I) or of any of the formulas as described herein, or of a compound as recited in any of the claims and described herein, or a salt or a stereoisomer thereof.

In some embodiments, provided herein is a method for treating cancer. The method includes administering to a patient in need thereof, a therapeutically effective amount of a compound of Formula (I′), Formula (I) or any of the formulas as described herein, a compound as recited in any of the claims and described herein, or a salt thereof. Examples of cancers include those whose growth may be inhibited using compounds of the disclosure and cancers typically responsive to immunotherapy.

In some embodiments, the present disclosure provides a method of enhancing, stimulating and/or increasing the immune response in a patient. The method includes administering to the patient in need thereof a therapeutically effective amount of a compound of Formula (I′), Formula (I) or any of the formulas as described herein, a compound or composition as recited in any of the claims and described herein, or a salt thereof.

Examples of cancers that are treatable using the compounds or combinations of the present disclosure include, but are not limited to, ewing sarcoma, cholangiocarcinoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva, Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers, especially metastatic cancers that express PD-L1.

In some embodiments, cancers treatable with compounds or combinations of the present disclosure include melanoma (e.g., metastatic malignant melanoma), renal cancer (e.g. clear cell carcinoma), prostate cancer (e.g. hormone refractory prostate adenocarcinoma), breast cancer, colon cancer and lung cancer (e.g. non-small cell lung cancer). Additionally, the disclosure includes refractory or recurrent malignancies whose growth may be inhibited using the compounds of the disclosure.

In some embodiments, cancers that are treatable using the compounds or combinations of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including relapsed or refractory NHL and recurrent follicular), Hodgkin lymphoma or multiple myeloma) and combinations of said cancers.

PD-1 pathway blockade with compounds of the present disclosure can also be used for treating infections such as viral, bacteria, fungus and parasite infections. The present disclosure provides a method for treating infections such as viral infections. The method includes administering to a patient in need thereof, a therapeutically effective amount of a compound of Formula (I′), Formula (I) or any of the formulas as described herein, a compound as recited in any of the claims and described herein, a salt thereof. Examples of viruses causing infections treatable by methods of the present disclosure include, but are not limit to, human immunodeficiency virus, human papillomavirus, influenza, hepatitis A, B, C or D viruses, adenovirus, poxvirus, herpes simplex viruses, human cytomegalovirus, severe acute respiratory syndrome virus, ebola virus, and measles virus. In some embodiments, viruses causing infections treatable by methods of the present disclosure include, but are not limit to, hepatitis (A, B, or C), herpes virus (e.g., VZV, HSV-1, HAV-6, HSV-II, and CMV, Epstein Barr virus), adenovirus, influenza virus, flaviviruses, echovirus, rhinovirus, coxsackie virus, cornovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

The present disclosure provides a method for treating bacterial infections. The method includes administering to a patient in need thereof, a therapeutically effective amount of a compound of Formula (I′), Formula (I) or any of the formulas as described herein, a compound as recited in any of the claims and described herein, or a salt thereof. Non-limiting examples of pathogenic bacteria causing infections treatable by methods of the disclosure include chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

The present disclosure provides a method for treating fungus infections. The method includes administering to a patient in need thereof, a therapeutically effective amount of a compound of Formula (I′), Formula (I) or any of the formulas as described herein, a compound as recited in any of the claims and described herein, or a salt thereof. Non-limiting examples of pathogenic fungi causing infections treatable by methods of the disclosure include Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

The present disclosure provides a method for treating parasite infections. The method includes administering to a patient in need thereof, a therapeutically effective amount of a compound of Formula (I′), Formula (I) or any of the formulas as described herein, a compound as recited in any of the claims and described herein, or a salt thereof. Non-limiting examples of pathogenic parasites causing infections treatable by methods of the disclosure include Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

Cancer cell growth and survival can be impacted by multiple signaling pathways. Thus, it is useful to combine different enzyme/protein/receptor inhibitors, exhibiting different preferences in the targets which they modulate the activities of, to treat such conditions. Targeting more than one signaling pathway (or more than one biological molecule involved in a given signaling pathway) may reduce the likelihood of drug-resistance arising in a cell population, and/or reduce the toxicity of treatment.

The compounds of the present disclosure can be used in combination with one or more other enzyme/protein/receptor inhibitors for the treatment of diseases, such as cancer or infections. Examples of cancers include solid tumors and liquid tumors, such as blood cancers. Examples of infections include viral infections, bacterial infections, fungus infections or parasite infections. For example, the compounds of the present disclosure can be combined with one or more inhibitors of the following kinases for the treatment of cancer: Akt1, Akt2, Akt3, TGF-βR, PKA, PKG, PKC, CaM-kinase, phosphorylase kinase, MEKK, ERK, MAPK, mTOR, EGFR, HER2, HER3, HER4, INS-R, IGF-1R, IR-R, PDGFαR, PDGFβR, CSFIR, KIT, FLK-II, KDR/FLK-1, FLK-4, fit-1, FGFR1, FGFR2, FGFR3, FGFR4, c-Met, Ron, Sea, TRKA, TRKB, TRKC, FLT3, VEGFR/Flt2, Flt4, EphA1, EphA2, EphA3, EphB2, EphB4, Tie2, Src, Fyn, Lck, Fgr, Btk, Fak, SYK, FRK, JAK, ABL, ALK and B-Raf. In some embodiments, the compounds of the present disclosure can be combined with one or more of the following inhibitors for the treatment of cancer or infections. Non-limiting examples of inhibitors that can be combined with the compounds of the present disclosure for treatment of cancer and infections include an FGFR inhibitor (FGFR1, FGFR2, FGFR3 or FGFR4, e.g., INCB54828, INCB62079 and INCB63904), a JAK inhibitor (JAK1 and/or JAK2, e.g., ruxolitinib, baricitinib or INCB39110), an IDO inhibitor (e.g., epacadostat and NLG919), an LSD1 inhibitor (e.g., INCB59872 and INCB60003), a TDO inhibitor, a PI3K-delta inhibitor, a PI3K-gamma inhibitor, such as PI3K-gamma selective inhibitor (e.g., INCB50797), a Pim inhibitor, a CSF1R inhibitor, a TAM receptor tyrosine kinases (Tyro-3, Axl, and Mer), an angiogenesis inhibitor, an interleukin receptor inhibitor, bromo and extra terminal family members inhibitors (for example, bromodomain inhibitors or BET inhibitors such as INCB54329 and INCB57643) and an adenosine receptor antagonist or combinations thereof.

Compounds of the present disclosure can be used in combination with one or more immune checkpoint inhibitors. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab or pembrolizumab. In some embodiments, the anti-PD1 antibody is pembrolizumab. In some embodiments, the anti PD-1 antibody is SHR-1210.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the inhibitor of an immune checkpoint molecule is an inhibitor of LAG3, e.g., an anti-LAG3 antibody. In some embodiments, the anti-LAG3 antibody is BMS-986016 or LAG525.

In some embodiments, the inhibitor of an immune checkpoint molecule is an inhibitor of GITR, e.g., an anti-GITR antibody. In some embodiments, the anti-GITR antibody is TRX518 or MK-4166.

In some embodiments, the inhibitor of an immune checkpoint molecule is an inhibitor of OX40, e.g., an anti-OX40 antibody or OX40L fusion protein. In some embodiments, the anti-OX40 antibody is MEDI0562. In some embodiments, the OX40L fusion protein is MEDI6383.

Compounds of the present disclosure can be used in combination with one or more agents for the treatment of diseases such as cancer. In some embodiments, the agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

The compounds of the present disclosure can further be used in combination with other methods of treating cancers, for example by chemotherapy, irradiation therapy, tumor-targeted therapy, adjuvant therapy, immunotherapy or surgery. Examples of immunotherapy include cytokine treatment (e.g., interferons, GM-CSF, G-CSF, IL-2), CRS-207 immunotherapy, cancer vaccine, monoclonal antibody, adoptive T cell transfer, oncolytic virotherapy and immunomodulating small molecules, including thalidomide or JAK1/2 inhibitor and the like. The compounds can be administered in combination with one or more anti-cancer drugs, such as a chemotherapeutics. Example chemotherapeutics include any of: abarelix, aldesleukin, alemtuzumab, alitretinoin, allopurinol, altretamine, anastrozole, arsenic trioxide, asparaginase, azacitidine, bevacizumab, bexarotene, baricitinib, bleomycin, bortezombi, bortezomib, busulfan intravenous, busulfan oral, calusterone, capecitabine, carboplatin, carmustine, cetuximab, chlorambucil, cisplatin, cladribine, clofarabine, cyclophosphamide, cytarabine, dacarbazine, dactinomycin, dalteparin sodium, dasatinib, daunorubicin, decitabine, denileukin, denileukin diftitox, dexrazoxane, docetaxel, doxorubicin, dromostanolone propionate, eculizumab, epirubicin, erlotinib, estramustine, etoposide phosphate, etoposide, exemestane, fentanyl citrate, filgrastim, floxuridine, fludarabine, fluorouracil, fulvestrant, gefitinib, gemcitabine, gemtuzumab ozogamicin, goserelin acetate, histrelin acetate, ibritumomab tiuxetan, idarubicin, ifosfamide, imatinib mesylate, interferon alfa 2a, irinotecan, lapatinib ditosylate, lenalidomide, letrozole, leucovorin, leuprolide acetate, levamisole, lomustine, meclorethamine, megestrol acetate, melphalan, mercaptopurine, methotrexate, methoxsalen, mitomycin C, mitotane, mitoxantrone, nandrolone phenpropionate, nelarabine, nofetumomab, oxaliplatin, paclitaxel, pamidronate, panitumumab, pegaspargase, pegfilgrastim, pemetrexed disodium, pentostatin, pipobroman, plicamycin, procarbazine, quinacrine, rasburicase, rituximab, ruxolitinib, sorafenib, streptozocin, sunitinib, sunitinib maleate, tamoxifen, temozolomide, teniposide, testolactone, thalidomide, thioguanine, thiotepa, topotecan, toremifene, tositumomab, trastuzumab, tretinoin, uracil mustard, valrubicin, vinblastine, vincristine, vinorelbine, vorinostat and zoledronate.

Other anti-cancer agent(s) include antibody therapeutics such as trastuzumab (Herceptin), antibodies to costimulatory molecules such as CTLA-4 (e.g., ipilimumab), 4-1BB, antibodies to PD-1 and PD-L1, or antibodies to cytokines (IL-10, TGF-β, etc.). Examples of antibodies to PD-1 and/or PD-L1 that can be combined with compounds of the present disclosure for the treatment of cancer or infections such as viral, bacteria, fungus and parasite infections include, but are not limited to, nivolumab, pembrolizumab, MPDL3280A, MEDI-4736 and SHR-1210.

In some embodiments, the anti-cancer agent is an alkylating agent, a proteasome inhibitor, a corticosteroid, or an immunomodulatory agent. Examples of an alkylating agent include cyclophosphamide (CY), melphalan (MEL), and bendamustine. In some embodiments, the proteasome inhibitor is carfilzomib. In some embodiments, the corticosteroid is dexamethasone (DEX). In some embodiments, the immunomodulatory agent is lenalidomide (LEN) or pomalidomide (POM).

Compounds of the present disclosure can be used in combination with one or more immune checkpoint inhibitors for the treatment of diseases, such as cancer or infections. Exemplary immune checkpoint inhibitors include inhibitors against immune checkpoint molecules such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, PD-1, PD-L1 and PD-L2. In some embodiments, the immune checkpoint molecule is a stimulatory checkpoint molecule selected from CD27, CD28, CD40, ICOS, OX40, GITR and CD137. In some embodiments, the immune checkpoint molecule is an inhibitory checkpoint molecule selected from A2AR, B7-H3, B7-H4, BTLA, CTLA-4, IDO, KIR, LAG3, PD-1, TIM3, and VISTA. In some embodiments, the compounds provided herein can be used in combination with one or more agents selected from KIR inhibitors, TIGIT inhibitors, LAIR1 inhibitors, CD160 inhibitors, 2B4 inhibitors and TGFR beta inhibitors.

In some embodiments, the inhibitor of an immune checkpoint molecule is anti-PD1 antibody, anti-PD-L1 antibody, or anti-CTLA-4 antibody.

In some embodiments, the inhibitor of an immune checkpoint molecule is an inhibitor of PD-1, e.g., an anti-PD-1 monoclonal antibody. In some embodiments, the anti-PD-1 monoclonal antibody is nivolumab, pembrolizumab (also known as MK-3475), pidilizumab, SHR-1210, PDR001, or AMP-224. In some embodiments, the anti-PD-1 monoclonal antibody is nivolumab or pembrolizumab. In some embodiments, the anti-PD1 antibody is pembrolizumab.

In some embodiments, the inhibitor of an immune checkpoint molecule is an inhibitor of PD-L1, e.g., an anti-PD-L1 monoclonal antibody. In some embodiments, the anti-PD-L1 monoclonal antibody is BMS-935559, MEDI4736, MPDL3280A (also known as RG7446), or MSB0010718C. In some embodiments, the anti-PD-L1 monoclonal antibody is MPDL3280A or MEDI4736.

In some embodiments, the inhibitor of an immune checkpoint molecule is an inhibitor of CTLA-4, e.g., an anti-CTLA-4 antibody. In some embodiments, the anti-CTLA-4 antibody is ipilimumab.

In some embodiments, the inhibitor of an immune checkpoint molecule is an inhibitor of LAG3, e.g., an anti-LAG3 antibody. In some embodiments, the anti-LAG3 antibody is BMS-986016 or LAG525.

In some embodiments, the inhibitor of an immune checkpoint molecule is an inhibitor of GITR, e.g., an anti-GITR antibody. In some embodiments, the anti-GITR antibody is TRX518 or MK-4166.

In some embodiments, the inhibitor of an immune checkpoint molecule is an inhibitor of OX40, e.g., an anti-OX40 antibody or OX40L fusion protein. In some embodiments, the anti-OX40 antibody is MEDI0562. In some embodiments, the OX40L fusion protein is MEDI6383.

The compounds of the present disclosure can further be used in combination with one or more anti-inflammatory agents, steroids, immunosuppressants or therapeutic antibodies.

The compounds of Formula (I′), Formula (I) or any of the formulas as described herein, a compound as recited in any of the claims and described herein, or salts thereof can be combined with another immunogenic agent, such as cancerous cells, purified tumor antigens (including recombinant proteins, peptides, and carbohydrate molecules), cells, and cells transfected with genes encoding immune stimulating cytokines. Non-limiting examples of tumor vaccines that can be used include peptides of melanoma antigens, such as peptides of gp100, MAGE antigens, Trp-2, MARTI and/or tyrosinase, or tumor cells transfected to express the cytokine GM-CSF.

The compounds of Formula (I′), Formula (I) or any of the formulas as described herein, a compound as recited in any of the claims and described herein, or salts thereof can be used in combination with a vaccination protocol for the treatment of cancer. In some embodiments, the tumor cells are transduced to express GM-CSF. In some embodiments, tumor vaccines include the proteins from viruses implicated in human cancers such as Human Papilloma Viruses (HPV), Hepatitis Viruses (HBV and HCV) and Kaposi's Herpes Sarcoma Virus (KHSV). In some embodiments, the compounds of the present disclosure can be used in combination with tumor specific antigen such as heat shock proteins isolated from tumor tissue itself. In some embodiments, the compounds of Formula (I′), Formula (I) or any of the formulas as described herein, a compound as recited in any of the claims and described herein, or salts thereof can be combined with dendritic cells immunization to activate potent anti-tumor responses.

The compounds of the present disclosure can be used in combination with bispecific macrocyclic peptides that target Fe alpha or Fe gamma receptor-expressing effectors cells to tumor cells. The compounds of the present disclosure can also be combined with macrocyclic peptides that activate host immune responsiveness.

The compounds of the present disclosure can be used in combination with bone marrow transplant for the treatment of a variety of tumors of hematopoietic origin.

The compounds of Formula (I′), Formula (I) or any of the formulas as described herein, a compound as recited in any of the claims and described herein, or salts thereof can be used in combination with vaccines, to stimulate the immune response to pathogens, toxins, and self antigens. Examples of pathogens for which this therapeutic approach may be particularly useful, include pathogens for which there is currently no effective vaccine, or pathogens for which conventional vaccines are less than completely effective. These include, but are not limited to, HIV, Hepatitis (A, B, & C), Influenza, Herpes, Giardia, Malaria, Leishmania, Staphylococcus aureus, Pseudomonas aeruginosa.

Viruses causing infections treatable by methods of the present disclosure include, but are not limit to human papillomavirus, influenza, hepatitis A, B, C or D viruses, adenovirus, poxvirus, herpes simplex viruses, human cytomegalovirus, severe acute respiratory syndrome virus, ebola virus, measles virus, herpes virus (e.g., VZV, HSV-1, HAV-6, HSV-II, and CMV, Epstein Barr virus), flaviviruses, echovirus, rhinovirus, coxsackie virus, cornovirus, respiratory syncytial virus, mumpsvirus, rotavirus, measles virus, rubella virus, parvovirus, vaccinia virus, HTLV virus, dengue virus, papillomavirus, molluscum virus, poliovirus, rabies virus, JC virus and arboviral encephalitis virus.

Pathogenic bacteria causing infections treatable by methods of the disclosure include, but are not limited to, chlamydia, rickettsial bacteria, mycobacteria, staphylococci, streptococci, pneumonococci, meningococci and conococci, klebsiella, proteus, serratia, pseudomonas, legionella, diphtheria, salmonella, bacilli, cholera, tetanus, botulism, anthrax, plague, leptospirosis, and Lyme's disease bacteria.

Pathogenic fungi causing infections treatable by methods of the disclosure include, but are not limited to, Candida (albicans, krusei, glabrata, tropicalis, etc.), Cryptococcus neoformans, Aspergillus (fumigatus, niger, etc.), Genus Mucorales (mucor, absidia, rhizophus), Sporothrix schenkii, Blastomyces dermatitidis, Paracoccidioides brasiliensis, Coccidioides immitis and Histoplasma capsulatum.

Pathogenic parasites causing infections treatable by methods of the disclosure include, but are not limited to, Entamoeba histolytica, Balantidium coli, Naegleriafowleri, Acanthamoeba sp., Giardia lambia, Cryptosporidium sp., Pneumocystis carinii, Plasmodium vivax, Babesia microti, Trypanosoma brucei, Trypanosoma cruzi, Leishmania donovani, Toxoplasma gondi, and Nippostrongylus brasiliensis.

When more than one pharmaceutical agent is administered to a patient, they can be administered simultaneously, separately, sequentially, or in combination (e.g., for more than two agents).

### IV. Formulation, Dosage Forms and Administration

When employed as pharmaceuticals, the compounds of the present disclosure can be administered in the form of pharmaceutical compositions. Thus the present disclosure provides a composition comprising a compound of Formula (I), Formula (I) or any of the formulas as described herein, a compound as recited in any of the claims and described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This invention also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the invention, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel KOOLV™) In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 100 mg to about 500 mg, of the active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present invention.

The tablets or pills of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present invention can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt % of the compound of the invention. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present invention can vary according to, e.g., the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. Labeled Compounds and Assay Methods

The compounds of the present disclosure can further be useful in investigations of biological processes in normal and abnormal tissues. Thus, another aspect of the present invention relates to labeled compounds of the invention (radio-labeled, fluorescent-labeled, etc.) that would be useful not only in imaging techniques but also in assays, both in vitro and in vivo, for localizing and quantitating PD-1 or PD-L1 protein in tissue samples, including human, and for identifying PD-L1 ligands by inhibition binding of a labeled compound. Accordingly, the present invention includes PD-1/PD-L1 binding assays that contain such labeled compounds.

The present invention further includes isotopically-substituted compounds of the disclosure. An “isotopically-substituted” compound is a compound of the invention where one or more atoms are replaced or substituted by an atom having an atomic mass or mass number different from the atomic mass or mass number typically found in nature (i.e., naturally occurring). It is to be understood that a “radio-labeled” compound is a compound that has incorporated at least one isotope that is radioactive (e.g., radionuclide). Suitable radionuclides that may be incorporated in compounds of the present invention include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. The radionuclide that is incorporated in the instant radio-labeled compounds will depend on the specific application of that radio-labeled compound. For example, for in vitro PD-L1 protein labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br will generally be most useful. In some embodiments the radionuclide is selected from the group consisting of ³H, ¹⁴C, ¹²⁵I, ³⁵S and ⁸²Br. Synthetic methods for incorporating radio-isotopes into organic compounds are known in the art.

Specifically, a labeled compound of the invention can be used in a screening assay to identify and/or evaluate compounds. For example, a newly synthesized or identified compound (i.e., test compound) which is labeled can be evaluated for its ability to bind a PD-L1 protein by monitoring its concentration variation when contacting with the PD-L1 protein, through tracking of the labeling. For example, a test compound (labeled) can be evaluated for its ability to reduce binding of another compound which is known to bind to a PD-L1 protein (i.e., standard compound). Accordingly, the ability of a test compound to compete with the standard compound for binding to the PD-L1 protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### VI. Kits

The present disclosure also includes pharmaceutical kits useful, e.g., in the treatment or prevention of diseases or disorders associated with the activity of PD-L1 including its interaction with other proteins such as PD-1 and B7-1 (CD80), such as cancer or infections, which include one or more containers containing a pharmaceutical composition comprising a therapeutically effective amount of a compound of Formula (I′), Formula (I), or any of the embodiments thereof. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the Examples have been found to inhibit the activity of PD-1/PD-L1 protein/protein interaction according to at least one assay described herein.

### EXAMPLES

Experimental procedures for compounds of the invention are provided below. Open Access Preparative LCMS Purification of some of the compounds prepared was performed on Waters mass directed fractionation systems. The basic equipment setup, protocols and control software for the operation of these systems have been described in detail in literature. See, e.g., Blom, “Two-Pump At Column Dilution Configuration for Preparative LC-MS”, K. Blom, J. Combi. Chem., 2002, 4, 295-301; Blom et al., “Optimizing Preparative LC-MS Configurations and Methods for Parallel Synthesis Purification”, J. Combi. Chem., 2003, 5, 670-83; and Blom et al., “Preparative LC-MS Purification: Improved Compound Specific Method Optimization”, J. Combi. Chem., 2004, 6, 874-883.

### Example 1: (2S)-1-[(6-{[(2-methylbiphenyl-3-yl)amino]carbonyl}pyridin-3-yl)methyl]piperidine-2-carboxylic acid

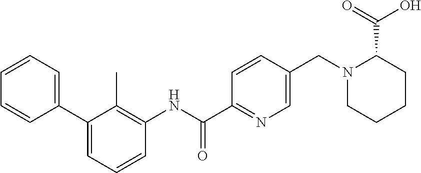 [SMILES: Cc1c(NC(=O)c2ccc(CN3CCCC[C@H]3C(=O)O)cn2)cccc1-c1ccccc1]

### Step 1: 2-methylbiphenyl-3-amine

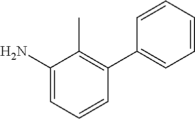

Tetrakis(triphenylphosphine)palladium(0) (300 mg, 0.3 mmol) was added to a mixture of 3-bromo-2-methylaniline (1000 mg, 5 mmol), phenylboronic acid (600 mg, 5 mmol), 1,4-dioxane (20 mL, 200 mmol) and water (3 mL, 200 mmol), and the mixture was allowed to stir at 100° C. overnight. After concentration, the residue was dissolved in dichloromethane (DCM) and washed with brine. The organic layer was dried and concentrated to afford the desired product as a brown solid, which was used in the next step without further purification. LC-MS calculated for C₁₃H₁₄N (M+H)⁺: m/z=184.1; found 184.1.

### Step 2: methyl 6-{[(2-methylbiphenyl-3-yl)amino]carbonyl}nicotinate

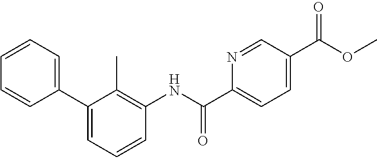

N,N-Diisopropylethylamine (200 μL, 1 mmol) was added to a mixture of 2-methylbiphenyl-3-amine (70 mg, 0.4 mmol), 5-(methoxycarbonyl)pyridine-2-carboxylic acid (Oakwood Chemical, cat #017196, 75 mg, 0.42 mmol), and N,N,N′,N′-tetramethyl-O-(7-azabenzotriazol-1-yl)uronium hexafluorophosphate (220 mg, 0.57 mmol) in N,N-dimethylformamide (2 mL, 20 mmol). The reaction mixture was allowed to stir at room temperature (rt) overnight. The reaction mixture was quenched with saturated aqueous NaHCO₃, and extracted with ethyl acetate (3×20 mL). The combined organic layers were washed with brine, dried over MgSO₄, filtered and concentrated under reduced pressure. The residue was purified by flash chromatography on a silica gel column with ethyl acetate in hexanes (0-35%) to afford the desired product. LC-MS calculated for C₂₁H₁₉N₂O₃ (M+H)⁺: m/z=347.1; found 347.1.

### Step 3: 5-(hydroxymethyl)-N-(2-methylbiphenyl-3-yl)pyridine-2-carboxamide

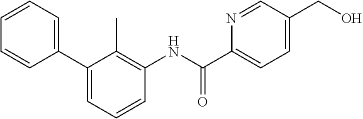

1.0 M Lithium tetrahydroaluminate in THF (0.6 mL, 0.6 mmol) was added to a THF (4.0 mL) solution of methyl 6-{[(2-methylbiphenyl-3-yl)amino]carbonyl}nicotinate (100 mg, 0.3 mmol) at 0° C. The reaction mixture was allowed to stir at 0° C. for 1 h to reach full conversion. The mixture was quenched with aqueous potassium sodium tartrate. The mixture was allowed to stir at rt for 2 h. The reaction mixture was extracted with ethyl acetate (3×10 mL). The combined organic layers were washed with brine, dried over MgSO₄, filtered and concentrated under reduced pressure. The mixture was dried, concentrated, and the residue was used in the next step without further purification. LC-MS calculated for C₂₀H₁₉N₂O₂ (M+H)⁺: m/z=319.1; found 319.0.

### Step 4: 5-formyl-N-(2-methylbiphenyl-3-yl)pyridine-2-carboxamide

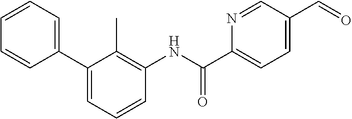

Dess-Martin periodinane (76 mg, 0.18 mmol) was added to a mixture of 5-(hydroxymethyl)-N-(2-methylbiphenyl-3-yl)pyridine-2-carboxamide (48 mg, 0.15 mmol), sodium bicarbonate (40 mg, 0.4 mmol), and methylene chloride (1 mL, 20 mmol), and the mixture was allowed to stir at rt for 2 h to reach full conversion. After concentration, the residue was dissolved in DCM and filtered. The organic layer was concentrated to afford desired product which was used in the next step without further purification. LC-MS calculated for C₂₀H₁₇N₂O₂ (M+H)⁺: m/z=317.1; found 317.0.

### Step 5: (2S)-1-[(6-{[(2-methylbiphenyl-3-yl)amino]carbonyl}pyridin-3-yl)methyl]piperidine-2-carboxylic acid

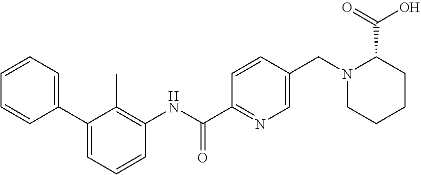

(2S)-Piperidine-2-carboxylic acid (20 mg, 0.1 mmol), sodium triacetoxyborohydride (60 mg, 0.3 mmol) and one drop of acetic acid were added to a methylene chloride (1 mL) solution of 5-formyl-N-(2-methylbiphenyl-3-yl)pyridine-2-carboxamide, and the mixture was allowed to stir at rt overnight. The reaction mixture was purified by prep-HPLC (pH=10, acetonitrile/water+NH₄OH) to give the desired product. LC-MS calculated for C₂₆H₂₈N₃O₃ (M+H)⁺: m/z=430.2; found 430.2.

### Example 2: 5-{[(2-hydroxyethyl)amino]methyl}-N-(2-methylbiphenyl-3-yl)pyridine-2-carboxamide

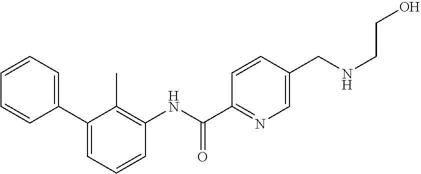

Ethanolamine (20 μL, 0.3 mmol) and sodium triacetoxyborohydride (60 mg, 0.3 mmol) were added to a methylene chloride (1 mL) solution of 5-formyl-N-(2-methylbiphenyl-3-yl)pyridine-2-carboxamide (Example 1, Step 4), and the mixture was allowed to stir at rt overnight. The reaction mixture was purified by prep-HPLC (pH=10, acetonitrile/water+NH₄OH) to give the desired product. LC-MS calculated for C₂₂H₂₄N₃O₂ (M+H)⁺: m/z=362.2; found 362.2.

### Example 3: (2S)-1-[(5-methyl-6-{[(2-methylbiphenyl-3-yl)amino]carbonyl}pyridin-3-yl)methyl]piperidine-2-carboxylic acid

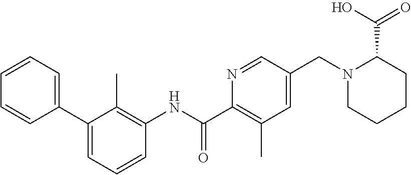

### Step 1: 5-bromo-3-methyl-N-(2-methylbiphenyl-3-yl)pyridine-2-carboxamide

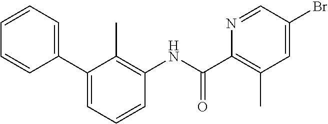

This compound was prepared using a similar procedure as described for Example 1, Step 2 with 5-bromo-2-carboxy-3-methylpyridine replacing 5-(methoxycarbonyl)pyridine-2-carboxylic acid. LC-MS calculated for C₂₀H₁₈BrN₂O (M+H)⁺: m/z=381.0; found: 381.1.

### Step 2: 3-methyl-N-(2-methylbiphenyl-3-yl)-5-vinylpyridine-2-carboxamide

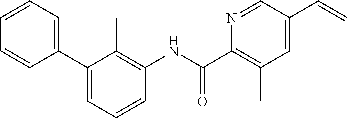

A mixture of 5-bromo-3-methyl-N-(2-methylbiphenyl-3-yl)pyridine-2-carboxamide (150 mg, 0.39 mmol), 1,1′-bis(diphenylphosphino)ferrocene]dichloropalladium(II) complexed with DCM (20 mg, 0.02 mmol), sodium bicarbonate, and 4,4,5,5-tetramethyl-2-vinyl-1,3,2-dioxaborolane (70 μL, 0.4 mmol) in dioxane (2.83 mL, 29.6 mmol) and water (1 mL, 60 mmol) was degassed and sealed. It was stirred at 95° C. overnight. The reaction mixture was diluted with EtOAc and washed with water and brine. The organic layer was separated and concentrated to afford desired product, which was used in the next step without further purification. LC-MS calculated for C₂₂H₂₁N₂O (M+H)⁺: m/z=329.2; found: 329.2.

### Step 3: 5-formyl-3-methyl-N-(2-methylbiphenyl-3-yl)pyridine-2-carboxamide

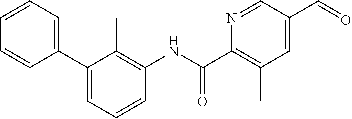

3-Methyl-N-(2-methylbiphenyl-3-yl)-5-vinylpyridine-2-carboxamide (100 mg, 0.4 mmol) was dissolved in 1,4-dioxane (6 mL, 80 mmol) and water (1 mL, 70 mmol). To this mixture was added a mixture of osmium tetraoxide (4% w/w in water, 0.38 mL, 0.06 mmol). After stirring for 5 min, sodium periodate (342 mg, 1.60 mmol) was added and the mixture was allowed to stir for 1 h to reach full conversion. The reaction mixture was extracted with ethyl acetate, and the combined organic layers were washed with brine, dried over MgSO₄, filtered and concentrated under reduced pressure to afford desired product, which was used in the next step without further purification. LC-MS calculated for C₂₁H₁₉N₂O₂ (M+H)⁺: m/z=331.1; found: 331.1.

### Step 4: (2S)-1-[(5-methyl-6-{[(2-methylbiphenyl-3-yl)amino]carbonyl}pyridin-3-yl)methyl]piperidine-2-carboxylic acid

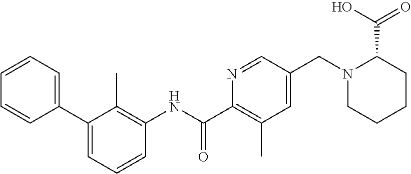

This compound was prepared using a similar procedure as described for Example 1, Step 5, with 5-formyl-3-methyl-N-(2-methylbiphenyl-3-yl)pyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₇H₃₀N₃O₃ (M+H)⁺: m/z=444.2; found 444.2.

### Example 4: 5-{[(2-hydroxyethyl)amino]methyl}-3-methyl-N-(2-methylbiphenyl-3-yl)pyridine-2-carboxamide

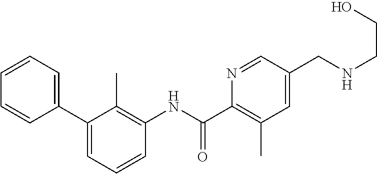

Ethanolamine (20 μL, 0.3 mmol) and sodium triacetoxyborohydride (60 mg, 0.3 mmol) were added to a methylene chloride (1 mL) solution of 5-formyl-3-methyl-N-(2-methylbiphenyl-3-yl)pyridine-2-carboxamide (Example 3, Step 3), and the mixture was allowed to stir at rt overnight. The reaction mixture was purified by prep-HPLC (pH=10, acetonitrile/water+NH₄OH) to give the desired product. LC-MS calculated for C₂₃H₂₆N₃O₂ (M+H)⁺: m/z=376.2; found 376.1.

### Example 5: (2S)-1-[(2-{[(2-methylbiphenyl-3-yl)amino]carbonyl}pyrimidin-5-yl)methyl]piperidine-2-carboxylic acid

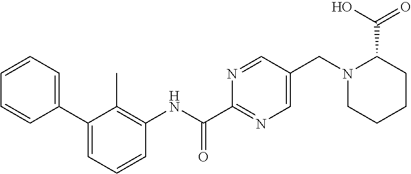

### Step 1: 5-bromo-N-(2-methylbiphenyl-3-yl)pyrimidine-2-carboxamide

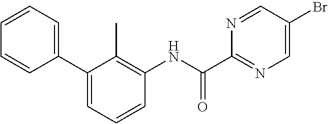

This compound was prepared using a similar procedure as described for Example 1, Step 2, with 5-bromopyrimidine-2-carboxylic acid (Ark Pharma, cat #AK-21808) replacing 5-(methoxycarbonyl)pyridine-2-carboxylic acid. LC-MS calculated for C₁₈H₁₅BrN₃O (M+H)⁺: m/z=368.0; found 368.0.

### Step 2: N-(2-methylbiphenyl-3-yl)-5-vinylpyrimidine-2-carboxamide

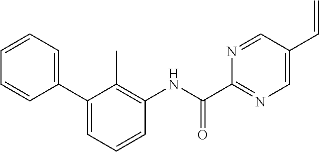

This compound was prepared using a similar procedure as described for Example 3, Step 2, with 5-bromo-N-(2-methylbiphenyl-3-yl)pyrimidine-2-carboxamide replacing 5-bromo-3-methyl-N-(2-methylbiphenyl-3-yl)pyridine-2-carboxamide. LC-MS calculated for C₂₀H₁₈N₃O (M+H)⁺: m/z=316.1; found 316.1.

### Step 3: 5-formyl-N-(2-methylbiphenyl-3-yl)pyrimidine-2-carboxamide

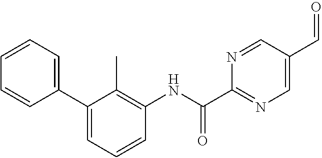

This compound was prepared using a similar procedure as described for Example 3, Step 3, with N-(2-methylbiphenyl-3-yl)-5-vinylpyrimidine-2-carboxamide replacing 3-methyl-N-(2-methylbiphenyl-3-yl)-5-vinylpyridine-2-carboxamide. LC-MS calculated for C₁₉H₁₆N₃O₂ (M+H)⁺: m/z=318.2; found 318.1.

### Step 4: (2S)-1-[(2-{[(2-methylbiphenyl-3-yl)amino]carbonyl}pyrimidin-5-yl)methyl]piperidine-2-carboxylic acid

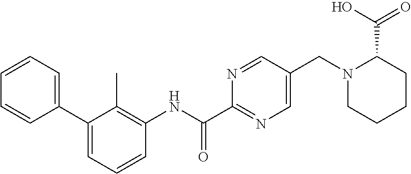

This compound was prepared using a similar procedure as described for Example 1, Step 5, with 5-formyl-N-(2-methylbiphenyl-3-yl)pyrimidine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₅H₂₇N₄O₃ (M+H)⁺: m/z=431.2; found 431.1.

### Example 6: 5-{[(2-hydroxyethyl)amino]methyl}-N-(2-methylbiphenyl-3-yl)pyrimidine-2-carboxamide

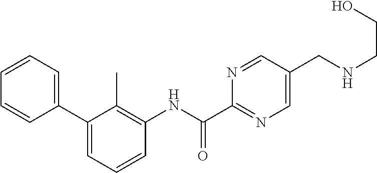 [SMILES: Cc1c(NC(=O)c2ncc(CNCCO)cn2)cccc1-c1ccccc1]

This compound was prepared using a similar procedure as described for Example 4 with 5-formyl-N-(2-methylbiphenyl-3-yl)pyrimidine-2-carboxamide (Example 5, Step 3) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₁H₂₃N₄O₂ (M+H)⁺: m/z=363.2; found 363.2.

### Example 7: (2S)-1-[(6-{[(2-cyanobiphenyl-3-yl)amino]carbonyl}pyridin-3-yl)methyl]piperidine-2-carboxylic acid

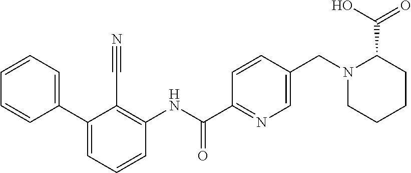 [SMILES: N#Cc1c(NC(=O)c2ccc(CN3CCCC[C@H]3C(=O)O)cn2)cccc1-c1ccccc1]

### Step 1: 3-aminobiphenyl-2-carbonitrile

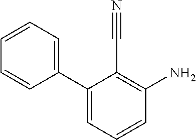 [SMILES: N#Cc1c(N)cccc1-c1ccccc1]

This compound was prepared using a similar procedure as described for Example 1, Step 1, with 2-amino-6-bromobenzonitrile (Astatech, cat #CL8148) replacing 3-bromo-2-methylaniline. LC-MS calculated for C₁₃H₁₁N₂(M+H)⁺: m/z=195.1; found 195.1.

### Step 2: 5-bromo-N-(2-cyanobiphenyl-3-yl)pyridine-2-carboxamide

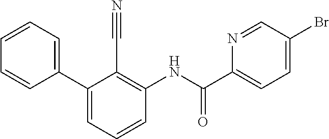 [SMILES: N#Cc1c(NC(=O)c2ccc(Br)cn2)cccc1-c1ccccc1]

N,N-Diisopropylethylamine (200 μL, 1 mmol) was added to a mixture of 3-aminobiphenyl-2-carbonitrile (80 mg, 0.4 mmol), 5-bromopyridine-2-carboxylic acid (Frontier Scientific cat #B1704, 96 mg, 0.47 mmol), and N,N,N′,N′-tetramethyl-O-(7-azabenzotriazol-1-yl)uronium hexafluorophosphate (250 mg, 0.65 mmol) in N,N-dimethylformamide (2 mL, 20 mmol). The reaction mixture was allowed to stir at rt overnight. The reaction mixture was quenched with saturated aqueous NaHCO₃, and extracted with ethyl acetate (3×20 mL). The combined organic layers were washed with brine, dried over MgSO₄, filtered and concentrated under reduced pressure. The residue was purified by flash chromatography on a silica gel column with ethyl acetate in hexanes (0-35%) to afford the desired product. LC-MS calculated for C₁₉H₁₃BrN₃O (M+H)⁺: m/z=378.0; found 378.0.

### Step 3: N-(2-cyanobiphenyl-3-yl)-5-vinylpyridine-2-carboxamide

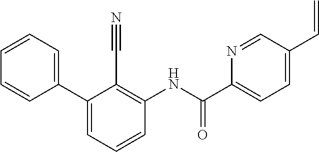 [SMILES: C=Cc1ccc(C(=O)Nc2cccc(-c3ccccc3)c2C#N)nc1]

This compound was prepared using a similar procedure as described for Example 3, Step 2, with 5-bromo-N-(2-cyanobiphenyl-3-yl)pyridine-2-carboxamide replacing 5-bromo-3-methyl-N-(2-methylbiphenyl-3-yl)pyridine-2-carboxamide. LC-MS calculated for C₂₁H₁₆N₃O (M+H)⁺: m/z=326.1; found 326.1.

### Step 4: N-(2-cyanobiphenyl-3-yl)-5-formylpyridine-2-carboxamide

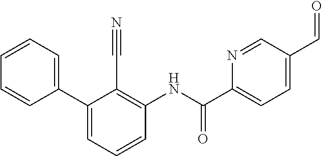 [SMILES: N#Cc1c(NC(=O)c2ccc(C=O)cn2)cccc1-c1ccccc1]

This compound was prepared using a similar procedure as described for Example 3, Step 3, with N-(2-cyanobiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₀H₁₄N₃O₂ (M+H)⁺: m/z=328.1; found 328.1.

### Step 5: (2S)-1-[(6-{[(2-cyanobiphenyl-3-yl)amino]carbonyl}pyridin-3-yl)methyl]piperidine-2-carboxylic acid

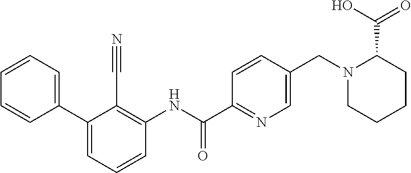 [SMILES: N#Cc1c(NC(=O)c2ccc(CN3CCCC[C@H]3C(=O)O)cn2)cccc1-c1ccccc1]

This compound was prepared using a similar procedure as described for Example 1, Step 5, with N-(2-cyanobiphenyl-3-yl)-5-formylpyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₆H₂₅N₄O₃ (M+H)⁺: m/z=441.2; found 441.1.

### Example 8: N-(2-cyanobiphenyl-3-yl)-5-{[(2-hydroxyethyl)amino]methyl}pyridine-2-carboxamide

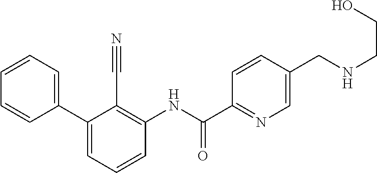 [SMILES: N#Cc1c(NC(=O)c2ccc(CNCCO)cn2)cccc1-c1ccccc1]

This compound was prepared using a similar procedure as described for Example 4 with N-(2-cyanobiphenyl-3-yl)-5-formylpyridine-2-carboxamide (Example 7, Step 4) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₂H₂₁N₄O₂ (M+H)⁺: m/z=373.2; found 373.2.

### Example 9: (2S)-1-{[6-({[2-cyano-3-(2,3-dihydro-1,4-benzodioxin-6-yl)phenyl]amino}carbonyl)pyridin-3-yl]methyl}piperidine-2-carboxylic acid

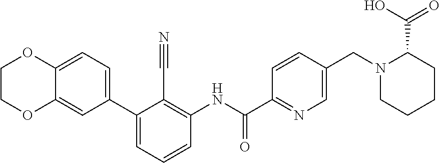 [SMILES: N#Cc1c(NC(=O)c2ccc(CN3CCCC[C@H]3C(=O)O)cn2)cccc1-c1ccc2c(c1)OCCO2]

### Step 1: 2-amino-6-(2,3-dihydro-1,4-benzodioxin-6-yl)benzonitrile

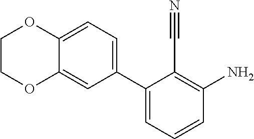 [SMILES: N#Cc1c(N)cccc1-c1ccc2c(c1)OCCO2]

This compound was prepared using a similar procedure as described for Example 1, Step 1, with 2,3-dihydro-1,4-benzodioxin-6-ylboronic acid (Sigma-Aldrich, cat #635995) replacing phenylboronic acid, and with 2-amino-6-bromobenzonitrile (Astatech, cat #CL8148) replacing 3-bromo-2-methylaniline. LC-MS calculated for C₁₅H₁₃N₂O₂ (M+H)⁺: m/z=253.1; found 253.1.

### Step 2: 5-bromo-N-[2-cyano-3-(2,3-dihydro-1,4-benzodioxin-6-yl)phenyl]pyridine-2-carboxamide

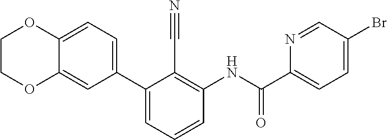 [SMILES: N#Cc1c(NC(=O)c2ccc(Br)cn2)cccc1-c1ccc2c(c1)OCCO2]

N,N-Diisopropylethylamine (200 μL, 1 mmol) was added to a mixture of 2-amino-6-(2,3-dihydro-1,4-benzodioxin-6-yl)benzonitrile (100 mg, 0.4 mmol), 5-bromopyridine-2-carboxylic acid (Frontier Scientific cat #B1704, 96 mg, 0.47 mmol), and N,N,N′,N′-tetramethyl-O-(7-azabenzotriazol-1-yl)uronium hexafluorophosphate (250 mg, 0.65 mmol) in N,N-dimethylformamide (2 mL, 20 mmol). The reaction mixture was allowed to stir at rt overnight. The reaction mixture was quenched with saturated aqueous NaHCO₃, and extracted with ethyl acetate (3×20 mL). The combined organic layers were washed with brine, dried over MgSO₄, filtered and concentrated under reduced pressure. The residue was purified by flash chromatography on a silica gel column with ethyl acetate in hexanes (0-35%) to afford the desired product. LC-MS calculated for C₂₁H₁₅BrN₃O₃ (M+H)⁺: m/z=436.0; found 436.0.

### Step 3: N-[2-cyano-3-(2,3-dihydro-1,4-benzodioxin-6-yl)phenyl]-5-vinylpyridine-2-carboxamide

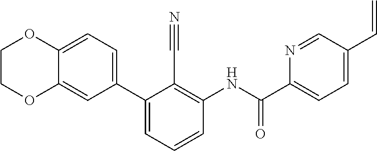 [SMILES: C=Cc1ccc(C(=O)Nc2cccc(-c3ccc4c(c3)OCCO4)c2C#N)nc1]

This compound was prepared using a similar procedure as described for Example 3, Step 2, with 5-bromo-N-[2-cyano-3-(2,3-dihydro-1,4-benzodioxin-6-yl)phenyl]pyridine-2-carboxamide replacing 5-bromo-3-methyl-N-(2-methylbiphenyl-3-yl)pyridine-2-carboxamide. LC-MS calculated for C₂₃H₁₈N₃O₃ (M+H)⁺: m/z=384.1; found 384.1.

### Step 4: N-[2-cyano-3-(2,3-dihydro-1,4-benzodioxin-6-yl)phenyl]-5-formylpyridine-2-carboxamide

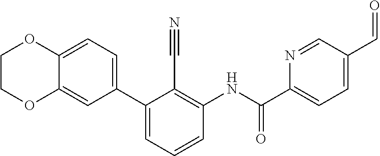 [SMILES: N#Cc1c(NC(=O)c2ccc(C=O)cn2)cccc1-c1ccc2c(c1)OCCO2]

This compound was prepared using a similar procedure as described for Example 3, Step 3, with N-[2-cyano-3-(2,3-dihydro-1,4-benzodioxin-6-yl)phenyl]-5-vinylpyridine-2-carboxamide replacing 3-methyl-N-(2-methylbiphenyl-3-yl)-5-vinylpyridine-2-carboxamide. LC-MS calculated for C₂₂H₁₆N₃O₄ (M+H)⁺: m/z=386.1; found 386.1.

### Step 5: (2S)-1-{[6-({[2-cyano-3-(2,3-dihydro-1,4-benzodioxin-6-yl)phenyl]amino}carbonyl)pyridin-3-yl]methyl}piperidine-2-carboxylic acid

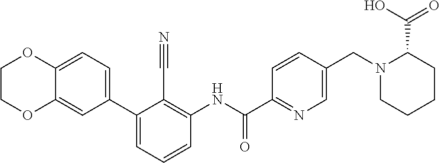 [SMILES: N#Cc1c(NC(=O)c2ccc(CN3CCCC[C@H]3C(=O)O)cn2)cccc1-c1ccc2c(c1)OCCO2]

This compound was prepared using a similar procedure as described for Example 1, Step 5, with N-[2-cyano-3-(2,3-dihydro-1,4-benzodioxin-6-yl)phenyl]-5-formylpyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₈H₂₇N₄O₅ (M+H)⁺: m/z=499.2; found 499.2.

### Example 10: N-(2-cyano-3-(2,3-dihydrobenzo[b][1,4]dioxin-6-yl)phenyl)-5-((2-hydroxyethylamino)methyl)picolinamide

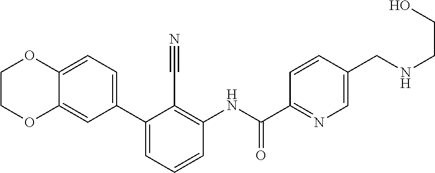 [SMILES: N#Cc1c(NC(=O)c2ccc(CNCCO)cn2)cccc1-c1ccc2c(c1)OCCO2]

This compound was prepared using a similar procedure as described for Example 4 with N-[2-cyano-3-(2,3-dihydro-1,4-benzodioxin-6-yl)phenyl]-5-formylpyridine-2-carboxamide (Example 9, Step 4) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₂H₂₁N₄O₂ (M+H)⁺: m/z=431.2; found 431.2.

### Example 11: 5-{[(2-hydroxyethyl)amino]methyl}-N-(2-methylbiphenyl-3-yl)pyrazine-2-carboxamide

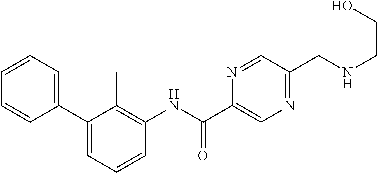 [SMILES: Cc1c(NC(=O)c2cnc(CNCCO)cn2)cccc1-c1ccccc1]

### Step 1: pyrazine-2,5-dicarboxylic acid

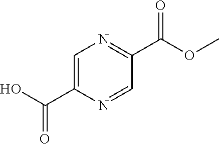

Sulfuric acid (19 μL, 0.36 mmol) was added to a methanol (1.2 mL, 29 mmol) solution of pyrazine-2,5-dicarboxylic acid (Ark Pharma cat #AK-76746, 100 mg, 0.6 mmol). Then the mixture was allowed to stir at 90° C. overnight. After cooling to rt, the mixture was diluted with methanol and basified with NaOH aq. Then the mixture was allowed to stir at rt for 5 h to reach ˜50% conversion to afford the mono-ester. Then the mixture was acidified with HCl aq, and extracted with DCM/iPrOH×3. The organic layer was dried and concentrated to afford desired product, which was used in the next step without further purification. LC-MS calculated for C₇H₇N₂O₄(M+H)⁺: m/z=183.0; found 183.0.

### Step 2: methyl 5-{[(2-methylbiphenyl-3-yl)amino]carbonyl}pyrazine-2-carboxylate

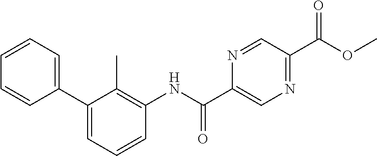 [SMILES: COC(=O)c1cnc(C(=O)Nc2cccc(-c3ccccc3)c2C)cn1]

This compound was prepared using a similar procedure as described for Example 1, Step 2, with pyrazine-2,5-dicarboxylic acid replacing 5-(methoxycarbonyl)pyridine-2-carboxylic acid. LC-MS calculated for C₂₀H₁₈N₃O₃ (M+H)⁺: m/z=348.1; found 348.1.

### Step 3: 5-(hydroxymethyl)-N-(2-methylbiphenyl-3-yl)pyrazine-2-carboxamide

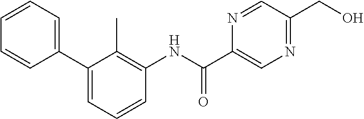 [SMILES: Cc1c(NC(=O)c2cnc(CO)cn2)cccc1-c1ccccc1]

This compound was prepared using a similar procedure as described for Example 1, Step 3, with methyl 5-{[(2-methylbiphenyl-3-yl)amino]carbonyl}pyrazine-2-carboxylate replacing methyl 6-{[(2-methylbiphenyl-3-yl)amino]carbonyl}nicotinate. Part of the reaction mixture was purified by prep-HPLC (pH=10, acetonitrile/water+NH₄OH) to give the desired product. LC-MS calculated for C₁₉H₁₈N₃O₂ (M+H)⁺: m/z=320.1; found 320.1.

### Step 4: 5-formyl-N-(2-methylbiphenyl-3-yl)pyrazine-2-carboxamide

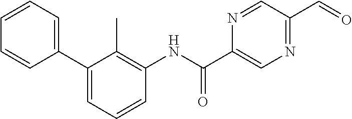 [SMILES: Cc1c(NC(=O)c2cnc(C=O)cn2)cccc1-c1ccccc1]

This compound was prepared using a similar procedure as described for Example 1, Step 4, with 5-(hydroxymethyl)-N-(2-methylbiphenyl-3-yl)pyrazine-2-carboxamide replacing 5-(hydroxymethyl)-N-(2-methylbiphenyl-3-yl)pyridine-2-carboxamid. LC-MS calculated for C₁₉H₁₆N₃O₂ (M+H)⁺: m/z=318.1; found 318.1.

### Step 5: 5-{[(2-hydroxyethyl)amino]methyl}-N-(2-methylbiphenyl-3-yl)pyrazine-2-carboxamide

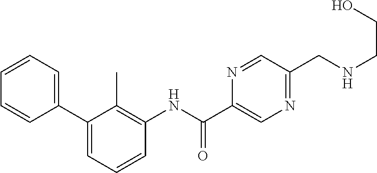 [SMILES: Cc1c(NC(=O)c2cnc(CNCCO)cn2)cccc1-c1ccccc1]

This compound was prepared using a similar procedure as described for Example 4 with 5-formyl-N-(2-methylbiphenyl-3-yl)pyrazine-2-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₁H₂₃N₄O₂ (M+H)⁺: m/z=363.2; found 363.2.

### Example 12: 6-{[(2-hydroxyethyl)amino]methyl}-N-(2-methylbiphenyl-3-yl)pyridazine-3-carboxamide

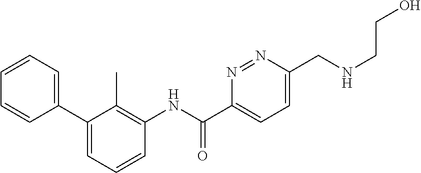 [SMILES: Cc1c(NC(=O)c2ccc(CNCCO)nn2)cccc1-c1ccccc1]

### Step 1: 6-(methoxycarbonyl)pyridazine-3-carboxylic acid

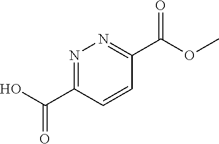

This compound was prepared using a similar procedure as described for Example 11, Step 1, with pyridazine-3,6-dicarboxylic acid (Astatech, cat #37156) replacing pyrazine-2,5-dicarboxylic acid. The reaction mixture was purified by prep-HPLC (pH=10, acetonitrile/water+NH₄OH) to give the desired product. LC-MS calculated for C₇H₇N₂O₄ (M+H)⁺: m/z=183.0; found 183.0.

### Step 2: methyl 6-{[(2-methylbiphenyl-3-yl)amino]carbonyl}pyridazine-3-carboxylate

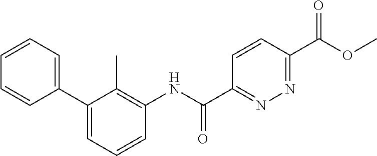 [SMILES: COC(=O)c1ccc(C(=O)Nc2cccc(-c3ccccc3)c2C)nn1]

This compound was prepared using a similar procedure as described for Example 1, Step 2, with 6-(methoxycarbonyl)pyridazine-3-carboxylic acid replacing 5-(methoxycarbonyl)pyridine-2-carboxylic acid. LC-MS calculated for C₂₁H₁₇N₃O₃ (M+H)⁺: m/z=348.1; found 348.1.

### Step 3: 6-(hydroxymethyl)-N-(2-methylbiphenyl-3-yl)pyridazine-3-carboxamide

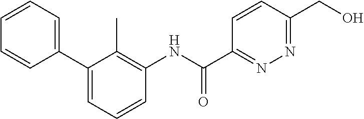 [SMILES: Cc1c(NC(=O)c2ccc(CO)nn2)cccc1-c1ccccc1]

This compound was prepared using a similar procedure as described for Example 1, Step 3, with methyl 6-{[(2-methylbiphenyl-3-yl)amino]carbonyl}pyridazine-3-carboxylate replacing methyl 6-{[(2-methylbiphenyl-3-yl)amino]carbonyl}nicotinate. LC-MS calculated for C₁₉H₁₈N₃O₂ (M+H)⁺: m/z=320.1; found 320.1.

### Step 4: 6-formyl-N-(2-methylbiphenyl-3-yl)pyridazine-3-carboxamide

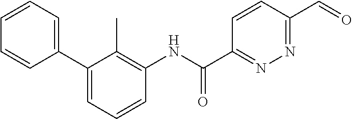 [SMILES: Cc1c(NC(=O)c2ccc(C=O)nn2)cccc1-c1ccccc1]

This compound was prepared using a similar procedure as described for Example 1, Step 4, with 6-(hydroxymethyl)-N-(2-methylbiphenyl-3-yl)pyridazine-3-carboxamide replacing 5-(hydroxymethyl)-N-(2-methylbiphenyl-3-yl)pyridine-2-carboxamide. LC-MS calculated for C₁₉H₁₆N₃O₂ (M+H)⁺: m/z=318.1; found 318.1.

### Step 5: 6-{[(2-hydroxyethyl)amino]methyl}-N-(2-methylbiphenyl-3-yl)pyridazine-3-carboxamide

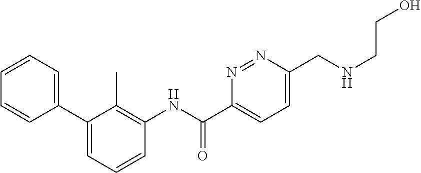 [SMILES: Cc1c(NC(=O)c2ccc(CNCCO)nn2)cccc1-c1ccccc1]

This compound was prepared using a similar procedure as described for Example 4 with 6-formyl-N-(2-methylbiphenyl-3-yl)pyridazine-3-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₁H₂₃N₄O₂ (M+H)⁺: m/z=363.2; found 363.2.

### Example 13: (2S)-1-[(2-methyl-6-{[(2-methylbiphenyl-3-yl)amino]carbonyl}pyridin-3-yl)methyl]piperidine-2-carboxylic acid

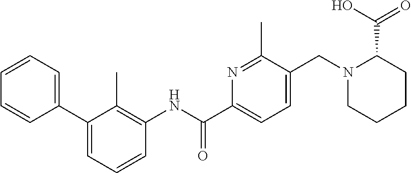 [SMILES: Cc1nc(C(=O)Nc2cccc(-c3ccccc3)c2C)ccc1CN1CCCC[C@H]1C(=O)O]

### Step 1: 5-bromo-6-methyl-N-(2-methylbiphenyl-3-yl)pyridine-2-carboxamide

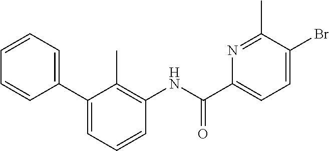 [SMILES: Cc1nc(C(=O)Nc2cccc(-c3ccccc3)c2C)ccc1Br]

This compound was prepared using a similar procedure as described for Example 1, Step 2, with 5-bromo-6-methylpyridine-2-carboxylic acid (Ark Pharma cat #Ak-61563) replacing 5-(methoxycarbonyl)pyridine-2-carboxylic acid. LC-MS calculated for C₂₀H₁₈BrN₂O (M+H)⁺: m/z=381.0; found 381.0.

### Step 2: 6-methyl-N-(2-methylbiphenyl-3-yl)-5-vinylpyridine-2-carboxamide

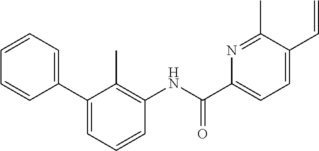 [SMILES: C=Cc1ccc(C(=O)Nc2cccc(-c3ccccc3)c2C)nc1C]

This compound was prepared using a similar procedure as described for Example 3, Step 2, with 5-bromo-6-methyl-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₂H₂₁N₂O (M+H)⁺: m/z=329.2; found 329.2.

### Step 3: 5-formyl-6-methyl-N-(2-methylbiphenyl-3-yl)pyridine-2-carboxamide

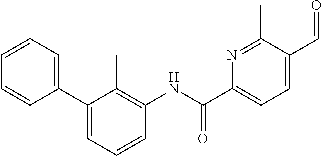 [SMILES: Cc1nc(C(=O)Nc2cccc(-c3ccccc3)c2C)ccc1C=O]

This compound was prepared using a similar procedure as described for Example 3, Step 3, with 6-methyl-N-(2-methylbiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₁H₁₉N₂O₂ (M+H)⁺: m/z=331.1; found 331.1.

### Step 4: (2S)-1-[(2-methyl-6-{[(2-methylbiphenyl-3-yl)amino]carbonyl}pyridin-3-yl)methyl]piperidine-2-carboxylic acid

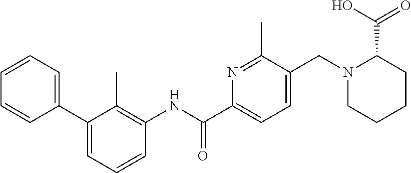 [SMILES: Cc1nc(C(=O)Nc2cccc(-c3ccccc3)c2C)ccc1CN1CCCC[C@H]1C(=O)O]

This compound was prepared using a similar procedure as described for Example 1, Step 5, with 5-formyl-6-methyl-N-(2-methylbiphenyl-3-yl)pyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₇H₃₀N₃O₃ (M+H)⁺: m/z=444.2; found 444.2.

### Example 14: 5-{[(2-hydroxyethyl)amino]methyl}-6-methyl-N-(2-methylbiphenyl-3-yl)pyridine-2-carboxamide

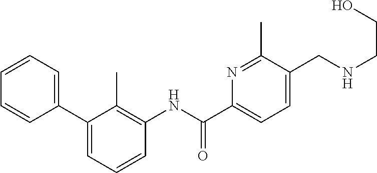 [SMILES: Cc1nc(C(=O)Nc2cccc(-c3ccccc3)c2C)ccc1CNCCO]

This compound was prepared using a similar procedure as described for Example 4 with 5-formyl-6-methyl-N-(2-methylbiphenyl-3-yl)pyridine-2-carboxamide (Example 13, Step 3) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₃H₂₆N₃O₂ (M+H)⁺: m/z=376.2; found 376.2.

### Example 15: (2S)-1-[(5-chloro-6-{[(2-methylbiphenyl-3-yl)amino]carbonyl}pyridin-3-yl)methyl]piperidine-2-carboxylic acid

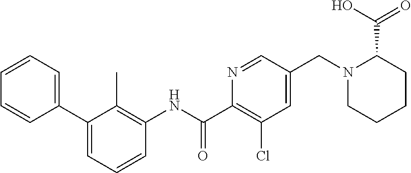 [SMILES: Cc1c(NC(=O)c2ncc(CN3CCCC[C@H]3C(=O)O)cc2Cl)cccc1-c1ccccc1]

### Step 1: 5-bromo-3-chloro-N-(2-methylbiphenyl-3-yl)pyridine-2-carboxamide

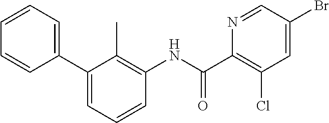 [SMILES: Cc1c(NC(=O)c2ncc(Br)cc2Cl)cccc1-c1ccccc1]

This compound was prepared using a similar procedure as described for Example 1, Step 2, with 5-bromo-3-chloropyridine-2-carboxylic acid (Ark Pharma cat #AK-53906) replacing 5-(methoxycarbonyl)pyridine-2-carboxylic acid. LC-MS calculated for C₁₉H₁₅BrClN₂O (M+H)⁺: m/z=401.0; found 401.1.

### Step 2: 3-chloro-N-(2-methylbiphenyl-3-yl)-5-vinylpyridine-2-carboxamide

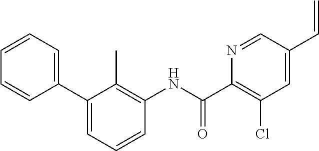 [SMILES: C=Cc1cnc(C(=O)Nc2cccc(-c3ccccc3)c2C)c(Cl)c1]

This compound was prepared using a similar procedure as described for Example 3, Step 2, with 5-bromo-3-chloro-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₁H₁₈ClN₂O (M+H)⁺: m/z=349.1; found 349.0.

### Step 3: 3-chloro-5-formyl-N-(2-methylbiphenyl-3-yl)pyridine-2-carboxamide

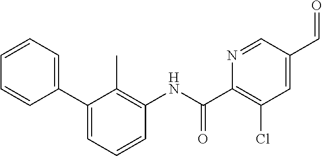 [SMILES: Cc1c(NC(=O)c2ncc(C=O)cc2Cl)cccc1-c1ccccc1]

This compound was prepared using a similar procedure as described for Example 3, Step 3, with 3-chloro-N-(2-methylbiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₀H₁₆C₁N₂O₂(M+H)⁺: m/z=351.1; found 351.0.

### Step 4: (2S)-1-[(5-chloro-6-{[(2-methylbiphenyl-3-yl)amino]carbonyl}pyridin-3-yl)methyl]piperidine-2-carboxylic acid

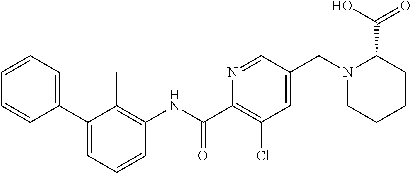 [SMILES: Cc1c(NC(=O)c2ncc(CN3CCCC[C@H]3C(=O)O)cc2Cl)cccc1-c1ccccc1]

This compound was prepared using a similar procedure as described for Example 1, Step 5 with 3-chloro-5-formyl-N-(2-methylbiphenyl-3-yl)pyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₆H₂₇C₁N₃O₃(M+H)⁺: m/z=464.2; found 464.0.

### Example 16: 3-chloro-5-{[(2-hydroxyethyl)amino]methyl}-N-(2-methylbiphenyl-3-yl)pyridine-2-carboxamide

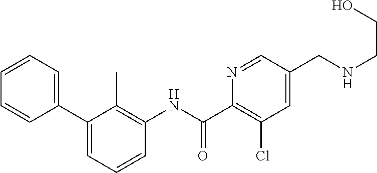 [SMILES: Cc1c(NC(=O)c2ncc(CNCCO)cc2Cl)cccc1-c1ccccc1]

This compound was prepared using a similar procedure as described for Example 4 with 3-chloro-5-formyl-N-(2-methylbiphenyl-3-yl)pyridine-2-carboxamide (Example 15, Step 3) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₂H₂₃C₁N₃O₂(M+H)⁺: m/z=396.1; found 396.1.

### Example 17: 3-fluoro-5-{[(2-hydroxyethyl)amino]methyl}-N-(2-methylbiphenyl-3-yl)pyridine-2-carboxamide

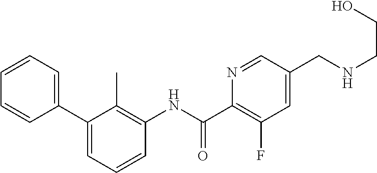 [SMILES: Cc1c(NC(=O)c2ncc(CNCCO)cc2F)cccc1-c1ccccc1]

### Step 1: 5-bromo-3-fluoro-N-(2-methylbiphenyl-3-yl)pyridine-2-carboxamide

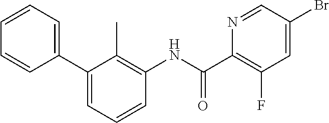 [SMILES: Cc1c(NC(=O)c2ncc(Br)cc2F)cccc1-c1ccccc1]

This compound was prepared using a similar procedure as described for Example 1, Step 2, with 5-bromo-3-fluoropyridine-2-carboxylic acid (Aldrich, cat #753483) replacing 5-(methoxycarbonyl)pyridine-2-carboxylic acid and prolong the reaction time to 3 days. LC-MS calculated for C₁₉H₁₅BrFN₂O (M+H)⁺: m/z=385.0; found 385.0.

### Step 2: 3-fluoro-N-(2-methylbiphenyl-3-yl)-5-vinylpyridine-2-carboxamide

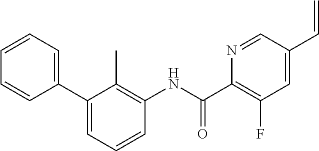 [SMILES: C=Cc1cnc(C(=O)Nc2cccc(-c3ccccc3)c2C)c(F)c1]

This compound was prepared using a similar procedure as described for Example 3, Step 2, with 5-bromo-3-fluoro-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₁H₁₈FN₂O (M+H)⁺: m/z=333.1; found 333.1.

### Step 3: 3-fluoro-5-formyl-N-(2-methylbiphenyl-3-yl)pyridine-2-carboxamide

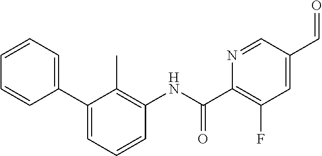 [SMILES: Cc1c(NC(=O)c2ncc(C=O)cc2F)cccc1-c1ccccc1]

This compound was prepared using a similar procedure as described for Example 3, Step 3, with 3-fluoro-N-(2-methylbiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₀H₁₆FN₂O₂(M+H)⁺: m/z=335.1; found 335.1.

### Step 4: 3-fluoro-5-{[(2-hydroxyethyl)amino]methyl}-N-(2-methylbiphenyl-3-yl)pyridine-2-carboxamide

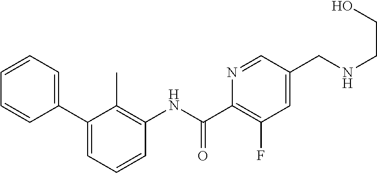 [SMILES: Cc1c(NC(=O)c2ncc(CNCCO)cc2F)cccc1-c1ccccc1]

This compound was prepared using a similar procedure as described for Example 4 with 3-fluoro-5-formyl-N-(2-methylbiphenyl-3-yl)pyridine-2-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₂H₂₃FN₃O₂(M+H)⁺: m/z=380.2; found 380.2.

### Example 18: 4-[(3-cyanobenzyl)oxy]-5-{[(2-hydroxyethyl)amino]methyl}-N-(2-methylbiphenyl-3-yl)pyridine-2-carboxamide

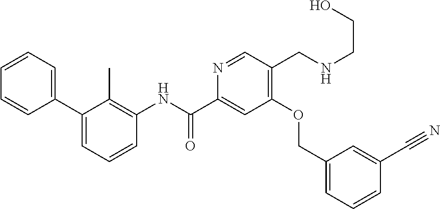 [SMILES: Cc1c(NC(=O)c2cc(OCc3cccc(C#N)c3)c(CNCCO)cn2)cccc1-c1ccccc1]

### Step 1: 5-bromo-4-chloro-N-(2-methylbiphenyl-3-yl)pyridine-2-carboxamide

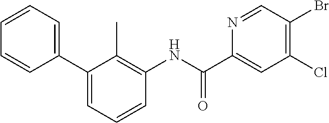 [SMILES: Cc1c(NC(=O)c2cc(Cl)c(Br)cn2)cccc1-c1ccccc1]

This compound was prepared using a similar procedure as described for Example 1, Step 2 with 5-bromo-4-chloropyridine-2-carboxylic acid (Ark Pharma, cat #AK-55136) replacing 5-(methoxycarbonyl)pyridine-2-carboxylic acid. LC-MS calculated for C₁₉H₁₅BrClN₂O (M+H)⁺: m/z=401.0; found 401.0.

### Step 2: 5-bromo-4-[(3-cyanobenzyl)oxy]-N-(2-methylbiphenyl-3-yl)pyridine-2-carboxamide

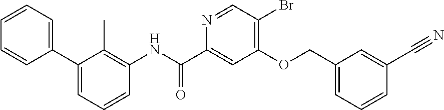 [SMILES: Cc1c(NC(=O)c2cc(OCc3cccc(C#N)c3)c(Br)cn2)cccc1-c1ccccc1]

Sodium hydride (60% w/w in oil, 9 mg, 0.4 mmol) was added to a mixture of 5-bromo-4-chloro-N-(2-methylbiphenyl-3-yl)pyridine-2-carboxamide (50 mg, 0.1 mmol), 3-(hydroxymethyl)benzonitrile (TCI cat #C1510, 40 mg, 0.30 mmol) and N,N-dimethylformamide (1 mL, 10 mmol) at rt. Then the mixture was allowed to stir at room temperature for 4 h to reach full conversion. The mixture was quenched with water and extracted with EtOAc×3, and the combined organic layers were dried and concentrated to afford desired product. LC-MS calculated for C₂₇H₂₁BrN₃O₂ (M+H)⁺: m/z=498.1; found 498.1.

### Step 3: 4-[(3-cyanobenzyl)oxy]-N-(2-methylbiphenyl-3-yl)-5-vinylpyridine-2-carboxamide

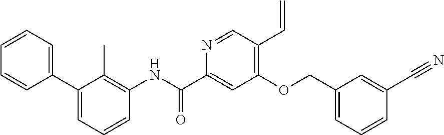 [SMILES: C=Cc1cnc(C(=O)Nc2cccc(-c3ccccc3)c2C)cc1OCc1cccc(C#N)c1]

This compound was prepared using a similar procedure as described for Example 3, Step 2, with 5-bromo-4-[(3-cyanobenzyl)oxy]-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₉H₂₄N₃O₂ (M+H)⁺: m/z=446.2; found 446.1.

### Step 4: 4-[(3-cyanobenzyl)oxy]-5-formyl-N-(2-methylbiphenyl-3-yl)pyridine-2-carboxamide

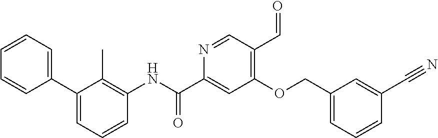 [SMILES: Cc1c(NC(=O)c2cc(OCc3cccc(C#N)c3)c(C=O)cn2)cccc1-c1ccccc1]

This compound was prepared using a similar procedure as described for Example 3, Step 3, with 4-[(3-cyanobenzyl)oxy]-N-(2-methylbiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₈H₂₂N₃O₃ (M+H)⁺: m/z=448.2; found 448.1.

### Step 5: 4-[(3-cyanobenzyl)oxy]-5-{[(2-hydroxyethyl)amino]methyl}-N-(2-methylbiphenyl-3-yl)pyridine-2-carboxamide

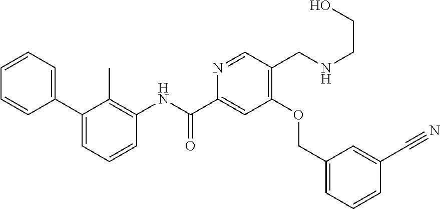 [SMILES: Cc1c(NC(=O)c2cc(OCc3cccc(C#N)c3)c(CNCCO)cn2)cccc1-c1ccccc1]

This compound was prepared using a similar procedure as described for Example 4 with 4-[(3-cyanobenzyl)oxy]-5-formyl-N-(2-methylbiphenyl-3-yl)pyridine-2-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₃₀H₂₉N₄O₃ (M+H)⁺: m/z=493.2; found 493.2.

### Example 19: 5-{[(2-hydroxyethyl)amino]methyl}-N-(2-methylbiphenyl-3-yl)-4-(pyridin-3-ylmethoxy)pyridine-2-carboxamide

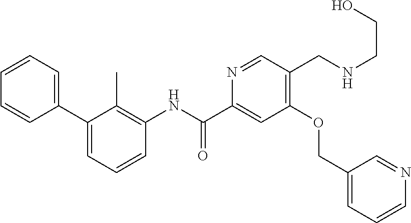 [SMILES: Cc1c(NC(=O)c2cc(OCc3cccnc3)c(CNCCO)cn2)cccc1-c1ccccc1]

### Step 1: 5-bromo-N-(2-methylbiphenyl-3-yl)-4-(pyridin-3-ylmethoxy)pyridine-2-carboxamide

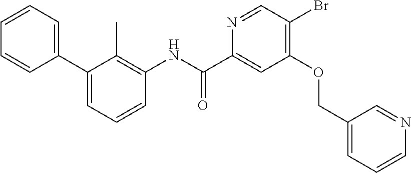 [SMILES: Cc1c(NC(=O)c2cc(OCc3cccnc3)c(Br)cn2)cccc1-c1ccccc1]

This compound was prepared using a similar procedure as described for Example 18, Step 2, with nicotinyl alcohol replacing 3-(hydroxymethyl)benzonitrile. LC-MS calculated for C₂₅H₂₁BrN₃O₂ (M+H)⁺: m/z=474.0; found 474.1.

### Step 2: N-(2-methylbiphenyl-3-yl)-4-(pyridin-3-ylmethoxy)-5-vinylpyridine-2-carboxamide

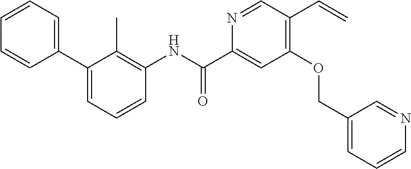 [SMILES: C=Cc1cnc(C(=O)Nc2cccc(-c3ccccc3)c2C)cc1OCc1cccnc1]

This compound was prepared using a similar procedure as described for Example 3, Step 2, with 5-bromo-N-(2-methylbiphenyl-3-yl)-4-(pyridin-3-ylmethoxy)pyridine-2-carboxamide replacing 5-bromo-3-methyl-N-(2-methylbiphenyl-3-yl)pyridine-2-carboxamide. LC-MS calculated for C₂₇H₂₄N₃O₂ (M+H)⁺: m/z=422.2; found 422.1.

### Step 3: 5-formyl-N-(2-methylbiphenyl-3-yl)-4-(pyridin-3-ylmethoxy)pyridine-2-carboxamide

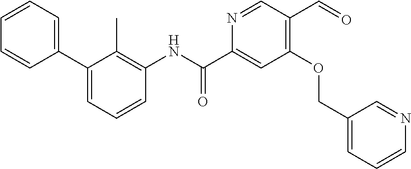 [SMILES: Cc1c(NC(=O)c2cc(OCc3cccnc3)c(C=O)cn2)cccc1-c1ccccc1]

This compound was prepared using a similar procedure as described for Example 3, Step 3, with N-(2-methylbiphenyl-3-yl)-4-(pyridin-3-ylmethoxy)-5-vinylpyridine-2-carboxamide replacing 3-methyl-N-(2-methylbiphenyl-3-yl)-5-vinylpyridine-2-carboxamide. LC-MS calculated for C₂₆H₂₂N₃O₃ (M+H)⁺: m/z=424.2; found 424.2.

### Step 4: 5-{[(2-hydroxyethyl)amino]methyl}-N-(2-methylbiphenyl-3-yl)-4-(pyridin-3-ylmethoxy)pyridine-2-carboxamide

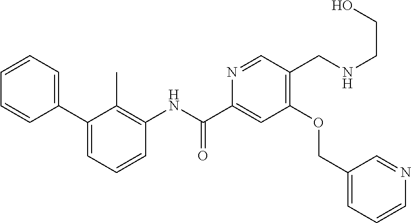 [SMILES: Cc1c(NC(=O)c2cc(OCc3cccnc3)c(CNCCO)cn2)cccc1-c1ccccc1]

This compound was prepared using a similar procedure as described for Example 4 with 5-formyl-N-(2-methylbiphenyl-3-yl)-4-(pyridin-3-ylmethoxy)pyridine-2-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product, which was further purified by prep-HPLC (pH=2, acetonitrile/water+TFA) to give the desired product as its TFA salt. LC-MS calculated for C₂₈H₂₉N₄O₃ (M+H)⁺: m/z=469.2; found 469.2.

### Example 20: 5-{[(2-hydroxyethyl)amino]methyl}-N-(2-methylbiphenyl-3-yl)-4-(pyridin-2-ylmethoxy)pyridine-2-carboxamide

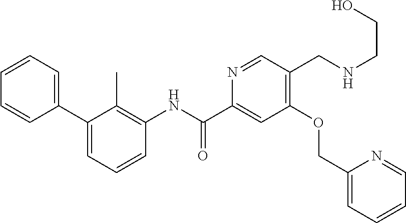 [SMILES: Cc1c(NC(=O)c2cc(OCc3ccccn3)c(CNCCO)cn2)cccc1-c1ccccc1]

### Step 1: 5-bromo-N-(2-methylbiphenyl-3-yl)-4-(pyridin-2-ylmethoxy)pyridine-2-carboxamide

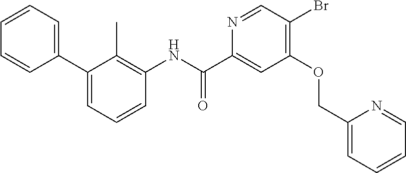 [SMILES: Cc1c(NC(=O)c2cc(OCc3ccccn3)c(Br)cn2)cccc1-c1ccccc1]

This compound was prepared using a similar procedure as described for Example 18, Step 2, with pyridin-2-ylmethanol replacing 3-(hydroxymethyl)benzonitrile. LC-MS calculated for C₂₅H₂₁BrN₃O₂ (M+H)⁺: m/z=474.0; found 474.1.

### Step 2: N-(2-methylbiphenyl-3-yl)-4-(pyridin-2-ylmethoxy)-5-vinylpyridine-2-carboxamide

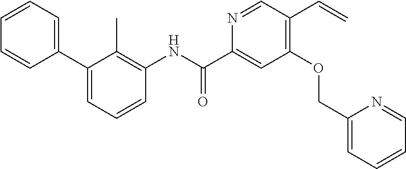 [SMILES: C=Cc1cnc(C(=O)Nc2cccc(-c3ccccc3)c2C)cc1OCc1ccccn1]

This compound was prepared using a similar procedure as described for Example 3, Step 2, with 5-bromo-N-(2-methylbiphenyl-3-yl)-4-(pyridin-2-ylmethoxy)pyridine-2-carboxamide replacing 5-bromo-3-methyl-N-(2-methylbiphenyl-3-yl)pyridine-2-carboxamide. LC-MS calculated for C₂₇H₂₄N₃O₂ (M+H)⁺: m/z=422.2; found 422.1.

### Step 3: 5-formyl-N-(2-methylbiphenyl-3-yl)-4-(pyridin-2-ylmethoxy)pyridine-2-carboxamide

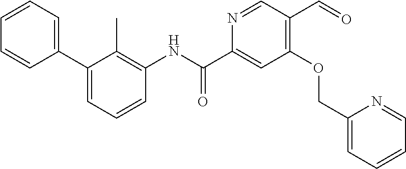 [SMILES: Cc1c(NC(=O)c2cc(OCc3ccccn3)c(C=O)cn2)cccc1-c1ccccc1]

This compound was prepared using a similar procedure as described for Example 3, Step 3, with N-(2-methylbiphenyl-3-yl)-4-(pyridin-2-ylmethoxy)-5-vinylpyridine-2-carboxamide replacing 3-methyl-N-(2-methylbiphenyl-3-yl)-5-vinylpyridine-2-carboxamide. LC-MS calculated for C₂₆H₂₂N₃O₃ (M+H)⁺: m/z=424.2; found 424.2.

### Step 4: 5-{[(2-hydroxyethyl)amino]methyl}-N-(2-methylbiphenyl-3-yl)-4-(pyridin-2-ylmethoxy)pyridine-2-carboxamide

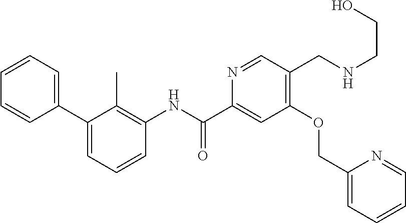 [SMILES: Cc1c(NC(=O)c2cc(OCc3ccccn3)c(CNCCO)cn2)cccc1-c1ccccc1]

This compound was prepared using a similar procedure as described for Example 4 with 5-formyl-N-(2-methylbiphenyl-3-yl)-4-(pyridin-2-ylmethoxy)pyridine-2-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₈H₂₉N₄O₃ (M+H)⁺: m/z=469.2; found 469.2.

### Example 21: 3-(dimethylamino)-5-{[(2-hydroxyethyl)amino]methyl}-N-(2-methylbiphenyl-3-yl)pyridine-2-carboxamide

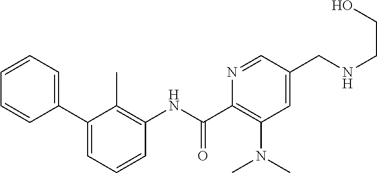 [SMILES: Cc1c(NC(=O)c2ncc(CNCCO)cc2N(C)C)cccc1-c1ccccc1]

### Step 1: 5-bromo-3-(dimethylamino)-N-(2-methylbiphenyl-3-yl)pyridine-2-carboxamide

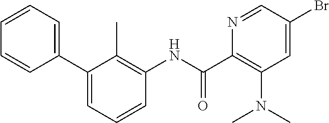 [SMILES: Cc1c(NC(=O)c2ncc(Br)cc2N(C)C)cccc1-c1ccccc1]

1.0 M Dimethylamine in THF (0.3 mL, 0.3 mmol) was added to 5-bromo-3-fluoro-N-(2-methylbiphenyl-3-yl)pyridine-2-carboxamide (Example 17, Step 1, 40 mg, 0.1 mmol) at rt, then the reaction was allowed to stir at rt overnight. The reaction was concentrated to afford desired product, which was used in the next step without further purification. LC-MS calculated for C₂₁H₂₁BrN₃O (M+H)⁺: m/z=410.1; found 410.1.

### Step 2: 3-(dimethylamino)-N-(2-methylbiphenyl-3-yl)-5-vinylpyridine-2-carboxamide

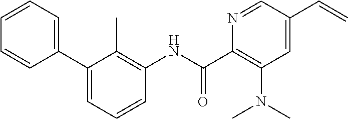 [SMILES: C=Cc1cnc(C(=O)Nc2cccc(-c3ccccc3)c2C)c(N(C)C)c1]

This compound was prepared using a similar procedure as described for Example 3, Step 2, with 5-bromo-3-(dimethylamino)-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₃H₂₄N₃O (M+H)⁺: m/z=358.2; found 358.1.

### Step 3: 3-(dimethylamino)-5-formyl-N-(2-methylbiphenyl-3-yl)pyridine-2-carboxamide

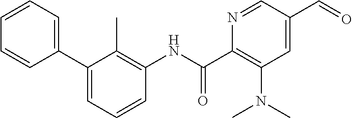 [SMILES: Cc1c(NC(=O)c2ncc(C=O)cc2N(C)C)cccc1-c1ccccc1]

This compound was prepared using a similar procedure as described for Example 3, Step 3, with 3-(dimethylamino)-N-(2-methylbiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₂H₂₂N₃O₂ (M+H)⁺: m/z=360.2; found 360.1.

### Step 4: 3-(dimethylamino)-5-{[(2-hydroxyethyl)amino]methyl}-N-(2-methylbiphenyl-3-yl)pyridine-2-carboxamide

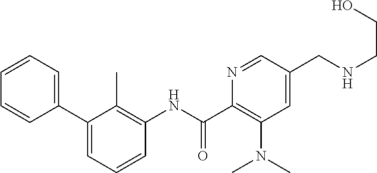 [SMILES: Cc1c(NC(=O)c2ncc(CNCCO)cc2N(C)C)cccc1-c1ccccc1]

This compound was prepared using a similar procedure as described for Example 4 with 3-(dimethylamino)-5-formyl-N-(2-methylbiphenyl-3-yl)pyridine-2-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₄H₂₉N₄O₂ (M+H)⁺: m/z=405.2; found 405.2.

### Example 22: (2S)-1-[(2-{[(2-methylbiphenyl-3-yl)amino]carbonyl}pyridin-4-yl)methyl]piperidine-2-carboxylic acid

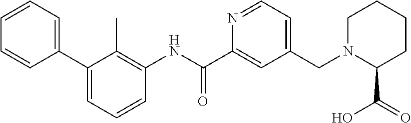 [SMILES: Cc1c(NC(=O)c2cc(CN3CCCC[C@H]3C(=O)O)ccn2)cccc1-c1ccccc1]

### Step 1: 4-bromo-N-(2-methylbiphenyl-3-yl)pyridine-2-carboxamide

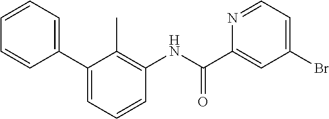 [SMILES: Cc1c(NC(=O)c2cc(Br)ccn2)cccc1-c1ccccc1]

This compound was prepared using a similar procedure as described for Example 1, Step 2, with 4-bromopyridine-2-carboxylic acid (Ark Pharm, cat #AK-23753) replacing 5-(methoxycarbonyl)pyridine-2-carboxylic acid. LC-MS calculated for C₁₉H₁₆BrN₂O (M+H)⁺: m/z=367.0; found 367.0.

### Step 2: N-(2-methylbiphenyl-3-yl)-4-vinylpyridine-2-carboxamide

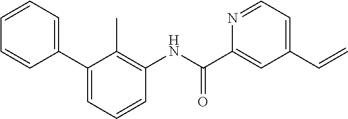 [SMILES: C=Cc1ccnc(C(=O)Nc2cccc(-c3ccccc3)c2C)c1]

This compound was prepared using a similar procedure as described for Example 3, Step 2, with 4-bromo-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₁H₁₉N₂O (M+H)⁺: m/z=315.1; found 315.2.

### Step 3: 4-formyl-N-(2-methylbiphenyl-3-yl)pyridine-2-carboxamide

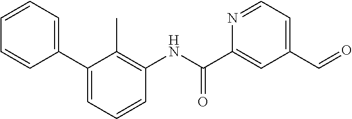 [SMILES: Cc1c(NC(=O)c2cc(C=O)ccn2)cccc1-c1ccccc1]

This compound was prepared using a similar procedure as described for Example 3, Step 3, with N-(2-methylbiphenyl-3-yl)-4-vinylpyridine-2-carboxamide replacing 3-methyl-N-(2-methylbiphenyl-3-yl)-5-vinylpyridine-2-carboxamide. LC-MS calculated for C₂₀H₁₇N₂O₂ (M+H)⁺: m/z=317.1; found 317.1.

### Step 4: (2S)-1-[(2-{[(2-methylbiphenyl-3-yl)amino]carbonyl}pyridin-4-yl)methyl]piperidine-2-carboxylic acid

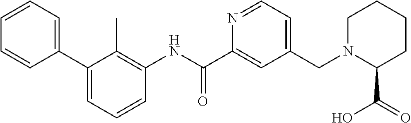 [SMILES: Cc1c(NC(=O)c2cc(CN3CCCC[C@H]3C(=O)O)ccn2)cccc1-c1ccccc1]

This compound was prepared using a similar procedure as described for Example 1, Step 5, with 4-formyl-N-(2-methylbiphenyl-3-yl)pyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₆H₂₈N₃O₃ (M+H)⁺: m/z=430.2; found 430.2.

### Example 23: 4-{[(2-hydroxyethyl)amino]methyl}-N-(2-methylbiphenyl-3-yl)pyridine-2-carboxamide

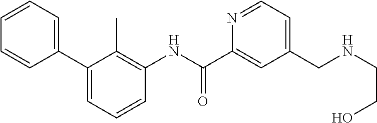 [SMILES: Cc1c(NC(=O)c2cc(CNCCO)ccn2)cccc1-c1ccccc1]

This compound was prepared using a similar procedure as described for Example 4 with 4-formyl-N-(2-methylbiphenyl-3-yl)pyridine-2-carboxamide (Example 25, Step 3) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product, which was concentrated and further purified by prep-HPLC (pH=2, acetonitrile/water+TFA) to give the desired product as its TFA salt. LC-MS calculated for C₂₂H₂₄N₃O₂ (M+H)⁺: m/z=362.2; found 362.2.

### Example 24: N-(2-methylbiphenyl-3-yl)-6-(pyridin-3-ylmethoxy)pyrimidine-4-carboxamide

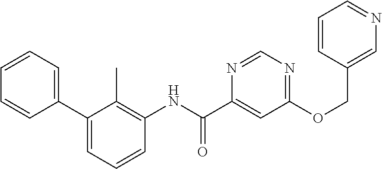 [SMILES: Cc1c(NC(=O)c2cc(OCc3cccnc3)ncn2)cccc1-c1ccccc1]

### Step 1: 6-(pyridin-3-ylmethoxy)pyrimidine-4-carboxylic acid

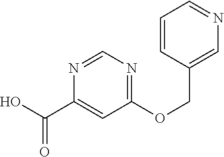 [SMILES: O=C(O)c1cc(OCc2cccnc2)ncn1]

Nicotinyl alcohol (73 μL, 0.76 mmol) was added to a suspension of sodium hydride (20 mg, 0.9 mmol) at rt, and the mixture was allowed to stir at rt for 1 h. Then a THF (1 mL) solution of 6-chloropyrimidine-4-carboxylic acid (Matrix Scientific cat #073471, 50 mg, 0.3 mmol) was added to the mixture, which was allowed to stir at rt for 3 h. The reaction was quenched with water and 1N HCl, the mixture was extracted with DCM/iPrOH. The organic layer was dried and concentrated to afford product which was used in the next step without further purification. LC-MS calculated for C₁₁H₁₀N₃O₃(M+H)⁺: m/z=232.1; found 232.2.

### Step 2: N-(2-methylbiphenyl-3-yl)-6-(pyridin-3-ylmethoxy)pyrimidine-4-carboxamide

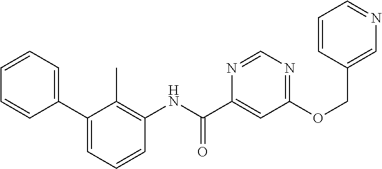 [SMILES: Cc1c(NC(=O)c2cc(OCc3cccnc3)ncn2)cccc1-c1ccccc1]

This compound was prepared using a similar procedure as described for Example 1, Step 2, with 6-(pyridin-3-ylmethoxy)pyrimidine-4-carboxylic acid replacing 5-(methoxycarbonyl)pyridine-2-carboxylic acid. LC-MS calculated for C₂₄H₂₁N₄O₂ (M+H)⁺: m/z=397.2; found 397.1.

### Example 25: 4-cyano-N-(2-methylbiphenyl-3-yl)pyridine-2-carboxamide

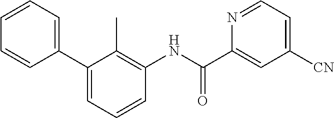 [SMILES: Cc1c(NC(=O)c2cc(C#N)ccn2)cccc1-c1ccccc1]

This compound was prepared using a similar procedure as described for Example 1, Step 2, with 4-cyanopyridine-2-carboxylic acid (Bionet Intermediates, cat #BB-0608) replacing 5-(methoxycarbonyl)pyridine-2-carboxylic acid. The reaction mixture was purified by prep-HPLC (pH=10, acetonitrile/water+NH₄OH) to give the desired product. LC-MS calculated for C₂₀H₁₆N₃O (M+H)⁺: m/z=314.1; found 314.1.

### Example 26: N-(2-methylbiphenyl-3-yl)pyrazine-2-carboxamide

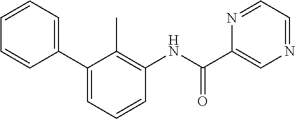 [SMILES: Cc1c(NC(=O)c2cnccn2)cccc1-c1ccccc1]

This compound was prepared using a similar procedure as described for Example 1, Step 2, with 2-pyrazinecarboxylic acid (Sigma-Aldrich, cat #P56100) replacing 5-(methoxycarbonyl)pyridine-2-carboxylic acid. The reaction mixture was purified by prep-HPLC (pH=10, acetonitrile/water+NH₄OH) to give the desired product. LC-MS calculated for C₁₈H₁₆N₃O (M+H)⁺: m/z=290.1; found 290.2.

### Example 27: N-(2-methylbiphenyl-3-yl)pyrimidine-4-carboxamide

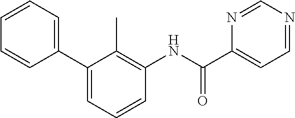 [SMILES: Cc1c(NC(=O)c2ccncn2)cccc1-c1ccccc1]

This compound was prepared using a similar procedure as described for Example 1, Step 2, with pyrimidine-4-carboxylic acid (VWR International, cat #101390) replacing 5-(methoxycarbonyl)pyridine-2-carboxylic acid. The reaction mixture was purified by prep-HPLC (pH=10, acetonitrile/water+NH₄OH) to give the desired product. LC-MS calculated for C₁₈H₁₆N₃O (M+H)⁺: m/z=290.1; found 290.2.

### Example 28: N-(2-methylbiphenyl-3-yl)pyrimidine-2-carboxamide

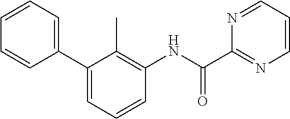 [SMILES: Cc1c(NC(=O)c2ncccn2)cccc1-c1ccccc1]

This compound was prepared using a similar procedure as described for Example 1, Step 2, with pyrimidine-2-carboxylic acid (Ark Pham, cat #AK-24353) replacing 5-(methoxycarbonyl)pyridine-2-carboxylic acid. The reaction mixture was purified by prep-HPLC (pH=10, acetonitrile/water+NH₄OH) to give the desired product. LC-MS calculated for C₁₈H₁₆N₃O (M+H)⁺: m/z=290.1; found 290.2.

### Example 29: N-(2-methylbiphenyl-3-yl)pyridazine-3-carboxamide

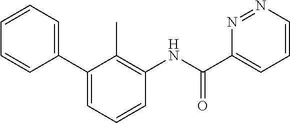 [SMILES: Cc1c(NC(=O)c2cccnn2)cccc1-c1ccccc1]

This compound was prepared using a similar procedure as described for Example 1, Step 2, with pyridazine-3-carboxylic acid (Ark Pham, cat #AK-28139) replacing 5-(methoxycarbonyl)pyridine-2-carboxylic acid. The reaction mixture was purified by prep-HPLC (pH=10, acetonitrile/water+NH₄OH) to give the desired product. LC-MS calculated for C₁₈H₁₆N₃O (M+H)⁺: m/z=290.1; found 290.1.

### Example 30: N-(2-methylbiphenyl-3-yl)-2-(pyridin-3-ylmethoxy)pyrimidine-4-carboxamide

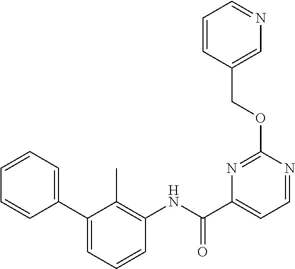 [SMILES: Cc1c(NC(=O)c2ccnc(OCc3cccnc3)n2)cccc1-c1ccccc1]

### Step 1: 2-(pyridin-3-ylmethoxy)pyrimidine-4-carboxylic acid

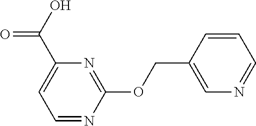 [SMILES: O=C(O)c1ccnc(OCc2cccnc2)n1]

Sodium hydride (20 mg, 0.9 mmol) was added to a tetrahydrofuran (2 mL, 20 mmol) solution of 2-chloropyrimidine-4-carboxylic acid (Ark Pharm, cat #AK-28365, 50 mg, 0.3 mmol) and nicotinyl alcohol (73 μL, 0.76 mmol), which was allowed to stir at 70° C. for 1 h to reach full conversion. The reaction mixture was quenched with water and extracted with DCM/iPrOH, and the organic layer was dried and concentrated to afford the desired product. Then the residue was used in the next step without further purification. LC-MS calculated for C₁₁H₁₀N₃O₃(M+H)⁺: m/z=232.1; found: 232.1.

### Step 2: N-(2-methylbiphenyl-3-yl)-2-(pyridin-3-ylmethoxy)pyrimidine-4-carboxamide

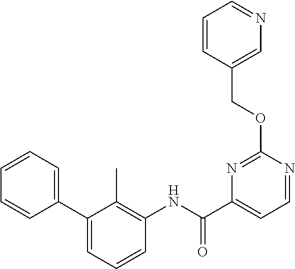 [SMILES: Cc1c(NC(=O)c2ccnc(OCc3cccnc3)n2)cccc1-c1ccccc1]

This compound was prepared using a similar procedure as described for Example 1, Step 2, with 2-(pyridin-3-ylmethoxy)pyrimidine-4-carboxylic acid replacing 5-(methoxycarbonyl)pyridine-2-carboxylic acid. The reaction mixture was purified by prep-HPLC (pH=10, acetonitrile/water+NH₄OH) to give the desired product. LC-MS calculated for C₂₄H₂₁N₄O₂ (M+H)⁺: m/z=397.2; found 397.2.

### Example 31: 5-{[(2-hydroxyethyl)amino]methyl}-N-(2-methylbiphenyl-3-yl)-4,4′-bipyridine-2-carboxamide

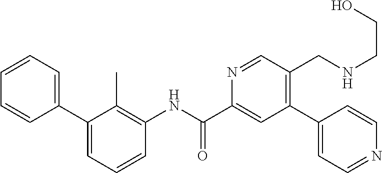 [SMILES: Cc1c(NC(=O)c2cc(-c3ccncc3)c(CNCCO)cn2)cccc1-c1ccccc1]

### Step 1: N-(2-methylbiphenyl-3-yl)-5-vinyl-4,4′-bipyridine-2-carboxamide

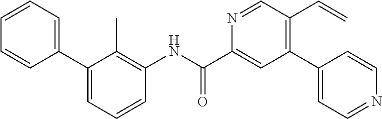 [SMILES: C=Cc1cnc(C(=O)Nc2cccc(-c3ccccc3)c2C)cc1-c1ccncc1]

A mixture of 5-bromo-4-chloro-N-(2-methylbiphenyl-3-yl)pyridine-2-carboxamide (Example 18, Step 1, 50 mg, 0.1 mmol), 4,4,5,5-tetramethyl-2-vinyl-1,3,2-dioxaborolane (20 μL, 0.1 mmol), and [1,1′-Bis(diphenylphosphino)ferrocene]dichloropalladium(II) complexed with dichloromethane (1:1) (6 mg, 0.008 mmol) in 1,4-dioxane (0.9 mL) and water (0.3 mL) was degassed and sealed. It was stirred at 90° C. for 1 h to reach full conversion. The reaction mixture was cooled and concentrated, followed by addition of 4-pyridinylboronic acid (30 mg, 0.2 mmol), cesium carbonate (100 mg, 0.4 mmol) and dichloro[1,1′-bis(dicyclohexylphosphino)ferrocene]palladium(II) (Pd-127: 9 mg, 0.01 mmol), tert-butyl alcohol (1 mL), and water (0.5 mL). The mixture was degassed with N₂ and heated at 100° C. for 2 h. After cooling to rt, the mixture was purified by 0 to 40% EtOAc in DCM. LC-MS calculated for C₂₆H₂₂N₃O (M+H)⁺: m/z=392.2; found: 392.1.

### Step 2: 5-formyl-N-(2-methylbiphenyl-3-yl)-4,4′-bipyridine-2-carboxamide

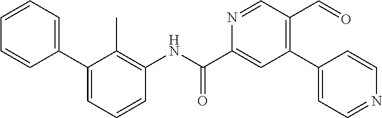 [SMILES: Cc1c(NC(=O)c2cc(-c3ccncc3)c(C=O)cn2)cccc1-c1ccccc1]

This compound was prepared using a similar procedure as described for Example 3, Step 3 with N-(2-methylbiphenyl-3-yl)-5-vinyl-4,4′-bipyridine-2-carboxamide replacing 3-methyl-N-(2-methylbiphenyl-3-yl)-5-vinylpyridine-2-carboxamide. LC-MS calculated for C₂₅H₂₀N₃O₂(M+H)⁺: m/z=394.2; found 394.1.

### Step 3: 5-{[(2-hydroxyethyl)amino]methyl}-N-(2-methylbiphenyl-3-yl)-4,4′-bipyridine-2-carboxamide

This compound was prepared using a similar procedure as described for Example 4 with 5-formyl-N-(2-methylbiphenyl-3-yl)-4,4′-bipyridine-2-carboxamide (Step 2) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product, which was concentrated and further purified by prep-HPLC (pH=2, acetonitrile/water+TFA) to give the desired product as its TFA salt. LC-MS calculated for C₂₇H₂₇N₄O₂ (M+H)⁺: m/z=439.2; found 439.2.

### Example 32: (2S)-1-[(4-methyl-6-{[(2-methylbiphenyl-3-yl)amino]carbonyl}pyridin-3-yl)methyl]piperidine-2-carboxylic acid

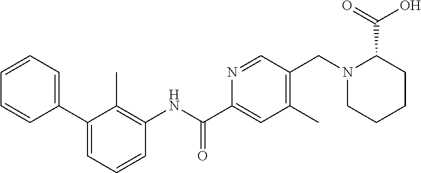 [SMILES: Cc1cc(C(=O)Nc2cccc(-c3ccccc3)c2C)ncc1CN1CCCC[C@H]1C(=O)O]

### Step 1: 5-bromo-4-methyl-N-(2-methylbiphenyl-3-yl)pyridine-2-carboxamide

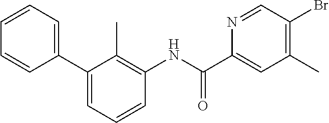 [SMILES: Cc1cc(C(=O)Nc2cccc(-c3ccccc3)c2C)ncc1Br]

This compound was prepared using a similar procedure as described for Example 1, Step 2 with 5-bromo-4-methylpyridine-2-carboxylic acid (ArkPharm, cat #AK-37510) replacing 5-(methoxycarbonyl)pyridine-2-carboxylic acid. LC-MS calculated for C₂₀H₁₈BrN₂O (M+H)⁺: m/z=381.0; found 381.0.

### Step 2: 4-methyl-N-(2-methylbiphenyl-3-yl)-5-vinylpyridine-2-carboxamide

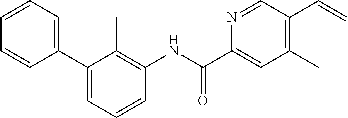 [SMILES: C=Cc1cnc(C(=O)Nc2cccc(-c3ccccc3)c2C)cc1C]

This compound was prepared using a similar procedure as described for Example 3, Step 2 with 5-bromo-4-methyl-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₂H₂₁N₂O (M+H)⁺: m/z=329.2; found 329.1.

### Step 3: 5-formyl-4-methyl-N-(2-methylbiphenyl-3-yl)pyridine-2-carboxamide

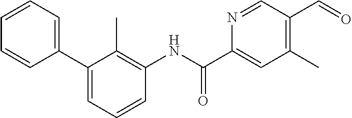 [SMILES: Cc1cc(C(=O)Nc2cccc(-c3ccccc3)c2C)ncc1C=O]

This compound was prepared using a similar procedure as described for Example 3, Step 3 with 4-methyl-N-(2-methylbiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₁H₁₉N₂O₂ (M+H)⁺: m/z=331.1; found 331.1.

### Step 4: (2S)-1-[(4-methyl-6-{[(2-methylbiphenyl-3-yl)amino]carbonyl}pyridin-3-yl)methyl]piperidine-2-carboxylic acid

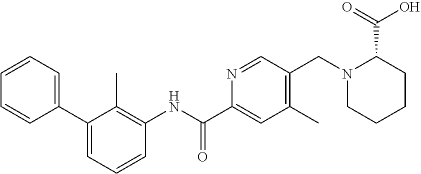

This compound was prepared using a similar procedure as described for Example 1, Step 5 with 5-formyl-4-methyl-N-(2-methylbiphenyl-3-yl)pyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₇H₃₀N₃O₃ (M+H)⁺: m/z=444.2; found 444.2.

### Example 33: 5-{[(2-hydroxyethyl)amino]methyl}-4-methyl-N-(2-methylbiphenyl-3-yl)pyridine-2-carboxamide

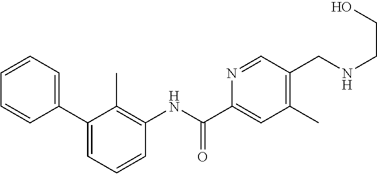

This compound was prepared using a similar procedure as described for Example 4 with 5-formyl-4-methyl-N-(2-methylbiphenyl-3-yl)pyridine-2-carboxamide (Example 32, Step 3) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₃H₂₆N₃O₂ (M+H)⁺: m/z=376.2; found 376.2.

### Example 34: 4-(cyclopropylmethoxy)-5-((2-hydroxyethylamino)methyl)-N-(2-methylbiphenyl-3-yl)picolinamide

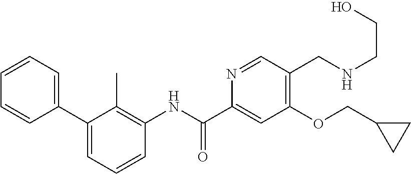

### Step 1: 5-bromo-4-(cyclopropylmethoxy)-N-(2-methylbiphenyl-3-yl)picolinamide

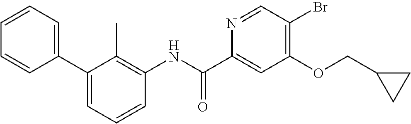

This compound was prepared using a similar procedure as described for Example 18, Step 2 with cyclopropylmethanol replacing 3-(hydroxymethyl)benzonitrile. LC-MS calculated for C₂₃H₂₂BrN₂O₂ (M+H)⁺: m/z=437.1; found 437.1.

### Step 2: 4-(cyclopropylmethoxy)-N-(2-methylbiphenyl-3-yl)-5-vinylpicolinamide

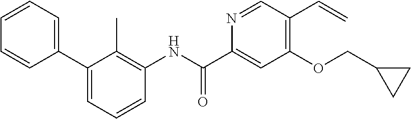

This compound was prepared using a similar procedure as described for Example 3, Step 2 with 5-bromo-4-(cyclopropylmethoxy)-N-(2-methylbiphenyl-3-yl)picolinamide replacing 5-bromo-3-methyl-N-(2-methylbiphenyl-3-yl)pyridine-2-carboxamide. LC-MS calculated for C₂₅H₂₄N₂O₂ (M+H)⁺: m/z=385.2; found 385.2.

### Step 3: 4-(cyclopropylmethoxy)-5-formyl-N-(2-methylbiphenyl-3-yl)picolinamide

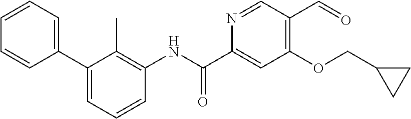

This compound was prepared using a similar procedure as described for Example 3, Step 3 with 4-(cyclopropylmethoxy)-N-(2-methylbiphenyl-3-yl)-5-vinylpicolinamide replacing 3-methyl-N-(2-methylbiphenyl-3-yl)-5-vinylpyridine-2-carboxamide. LC-MS calculated for C₂₄H₂₃N₂O₃ (M+H)⁺: m/z=387.2; found 387.2.

### Step 4: 4-(cyclopropylmethoxy)-5-((2-hydroxyethylamino)methyl)-N-(2-methylbiphenyl-3-yl)picolinamide

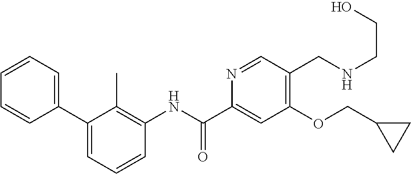

This compound was prepared using a similar procedure as described for Example 4 with 4-(cyclopropylmethoxy)-5-formyl-N-(2-methylbiphenyl-3-yl)picolinamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₆H₃₀N₃O₃ (M+H)⁺: m/z=432.2; found 432.2.

### Example 35: 5-{[(2-hydroxyethyl)amino]methyl}-4-methoxy-N-(2-methylbiphenyl-3-yl)pyridine-2-carboxamide

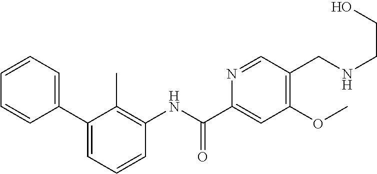

### Step 1: 5-bromo-4-methoxy-N-(2-methylbiphenyl-3-yl)pyridine-2-carboxamide

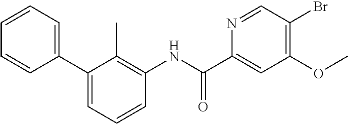

Mixture of sodium methoxide (80 μL, 0.4 mmol) was added to 5-bromo-4-chloro-N-(2-methylbiphenyl-3-yl)pyridine-2-carboxamide (Example 18, Step 1, 50 mg, 0.1 mmol) at rt. Then the mixture was allowed to stir at rt for 4 h to reach full conversion. The reaction was quenched with water and extracted with EtOAc, and the organic layer was dried and concentrated to afford desired product which was used for next step without further purification. LC-MS calculated for C₂₀H₁₈BrN₂O₂ (M+H)⁺: m/z=397.0; found 397.0.

### Step 2: 4-methoxy-N-(2-methylbiphenyl-3-yl)-5-vinylpyridine-2-carboxamide

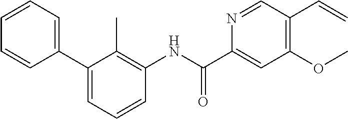

This compound was prepared using a similar procedure as described for Example 3, Step 2 with 5-bromo-4-methoxy-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₂H₂₁N₂O₂ (M+H)⁺: m/z=345.2; found 345.1.

### Step 3: 5-formyl-4-methoxy-N-(2-methylbiphenyl-3-yl)pyridine-2-carboxamide

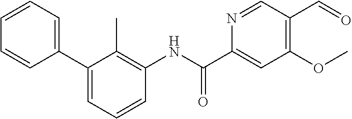

This compound was prepared using a similar procedure as described for Example 3, Step 3 with 4-methoxy-N-(2-methylbiphenyl-3-yl)-5-vinylpyridine-2-carboxamide replacing 3-methyl-N-(2-methylbiphenyl-3-yl)-5-vinylpyridine-2-carboxamide. LC-MS calculated for C₂₁H₁₉N₂O₃ (M+H)⁺: m/z=347.1; found 347.1.

### Step 4: 5-{[(2-hydroxyethyl)amino]methyl}-4-methoxy-N-(2-methylbiphenyl-3-yl)pyridine-2-carboxamide

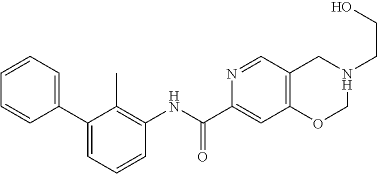

This compound was prepared using a similar procedure as described for Example 4 with 5-formyl-4-methoxy-N-(2-methylbiphenyl-3-yl)pyridine-2-carboxamide replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₃H₂₆N₃O₃ (M+H)⁺: m/z=392.2; found 392.2.

### Example 36: (2S)-1-[(5-methyl-2-{[(2-methylbiphenyl-3-yl)amino]carbonyl}pyridin-4-yl)methyl]piperidine-2-carboxylic acid

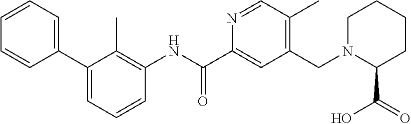

### Step 1: 4-bromo-5-methyl-N-(2-methylbiphenyl-3-yl)pyridine-2-carboxamide

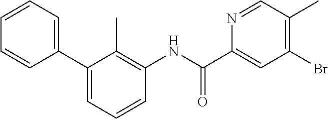

This compound was prepared using a similar procedure as described for Example 1, Step 2 with 4-bromo-5-methylpyridine-2-carboxylic acid (ArkPharm, cat #AK-37510) replacing 5-(methoxycarbonyl)pyridine-2-carboxylic acid. LC-MS calculated for C₂₀H₁₈BrN₂O (M+H)⁺: m/z=381.0; found 381.0.

### Step 2: 5-methyl-N-(2-methylbiphenyl-3-yl)-4-vinylpyridine-2-carboxamide

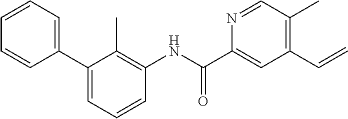

This compound was prepared using a similar procedure as described for Example 3, Step 2 with 4-bromo-5-methyl-N-(2-methylbiphenyl-3-yl)pyridine-2-carboxamide replacing 5-bromo-3-methyl-N-(2-methylbiphenyl-3-yl)pyridine-2-carboxamide. LC-MS calculated for C₂₂H₂₁N₂O (M+H)⁺: m/z=329.1; found 329.2.

### Step 3: 4-formyl-5-methyl-N-(2-methylbiphenyl-3-yl)pyridine-2-carboxamide

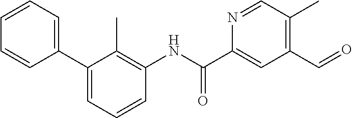 [SMILES: Cc1cnc(C(=O)Nc2cccc(-c3ccccc3)c2C)cc1C=O]

This compound was prepared using a similar procedure as described for Example 3, Step 3 with 5-methyl-N-(2-methylbiphenyl-3-yl)-4-vinylpyridine-2-carboxamide replacing 3-methyl-N-(2-methylbiphenyl-3-yl)-5-vinylpyridine-2-carboxamide. LC-MS calculated for C₂₁H₁₉N₂O₂(M+H)⁺: m/z=331.1; found 331.1.

### Step 4: (2S)-1-[(5-methyl-2-{[(2-methylbiphenyl-3-yl)amino]carbonyl}pyridin-4-yl)methyl]piperidine-2-carboxylic acid

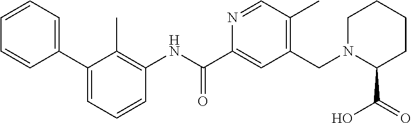 [SMILES: Cc1cnc(C(=O)Nc2cccc(-c3ccccc3)c2C)cc1CN1CCCC[C@H]1C(=O)O]

This compound was prepared using a similar procedure as described for Example 1, Step 5 with 4-formyl-5-methyl-N-(2-methylbiphenyl-3-yl)pyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₇H₃₀N₃O₃ (M+H)⁺: m/z=444.2; found 444.2.

### Example 37: 4-{[(2-hydroxyethyl)amino]methyl}-5-methyl-N-(2-methylbiphenyl-3-yl)pyridine-2-carboxamide

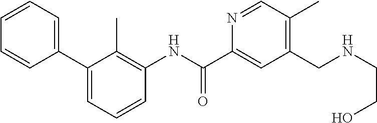 [SMILES: Cc1cnc(C(=O)Nc2cccc(-c3ccccc3)c2C)cc1CNCCO]

This compound was prepared using a similar procedure as described for Example 4 with 4-formyl-5-methyl-N-(2-methylbiphenyl-3-yl)pyridine-2-carboxamide (Example 36, Step 3) replacing 5-formyl-3-methyl-N-(2-methylbiphenyl-3-yl)pyridine-2-carboxamide. The reaction mixture was purified by prep-HPLC (pH=2, acetonitrile/water+TFA) to give the desired product as its TFA salt. LC-MS calculated for C₂₃H₂₆N₃O₂ (M+H)⁺: m/z=376.2; found 376.2.

### Example 38: N-(2-cyano-3-cyclohex-1-en-1-ylphenyl)-5-{[(2-hydroxyethyl)amino]methyl}pyridine-2-carboxamide

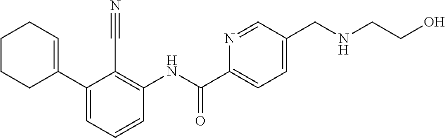 [SMILES: N#Cc1c(NC(=O)c2ccc(CNCCO)cn2)cccc1C1=CCCCC1]

### Step 1: methyl 5-vinylpyridine-2-carboxylate

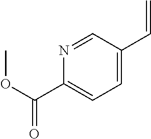

Sodium carbonate (1200 mg, 12 mmol), [1,1′-bis(diphenylphosphino)ferrocene]dichloropalladium(II) complexed with dichloromethane [PdCl₂(dppf), 100 mg, 0.1 mmol], 1,4-dioxane (15 mL), water (3 mL), 4,4,5,5-tetramethyl-2-vinyl-1,3,2-dioxaborolane (628 μL, 3.70 mmol) were added to 5-bromopyridine-2-carboxylic acid methyl ester (Combi-Blocks, cat #CA-4117, 800 mg, 4 mmol), and degassed with N₂. The mixture was heated at 90° C. for 2 h to reach full conversion. The mixture was purified with 0 to 35% EtOAc in Hexanes to afford the desired product as a white solid.

### Step 2: 5-vinylpyridine-2-carboxylic acid

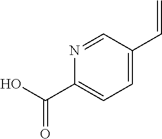

3 mL 1M NaOH solution (3 mmol) was added to methyl 5-vinylpyridine-2-carboxylate and heated at 45° C. for 2 h to reach full conversion. The mixture was concentrated to afford desired product as a white solid, which was used for next step without further purification.

### Step 3: N-(3-bromo-2-cyanophenyl)-5-vinylpyridine-2-carboxamide

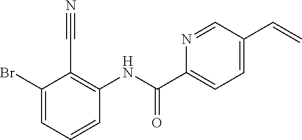 [SMILES: C=Cc1ccc(C(=O)Nc2cccc(Br)c2C#N)nc1]

2.0 M oxalyl chloride in DCM (4.7 mL, 9.3 mmol), N,N-dimethylformamide (30 μL, 0.3 mmol) was added to 5-vinylpyridine-2-carboxylic acid (522 mg, 3.50 mmol) under N₂ at 0° C. The mixture was allowed to stir at rt for 2 h, then the mixture was concentrated under reduced pressure and the residue was used for next step without further purification. 2-amino-6-bromobenzonitrile (ArkPharm, AK-36350, 690 mg, 3.5 mmol) and methylene chloride (10 mL) were added to the residue, followed by addition of triethylamine (1000 μL, 10 mmol) at 0° C. drop-wise. The mixture was allowed to stir at rt for 1 h to reach full conversion. The mixture was concentrated and purified by 0 to 50% EtOAc in Hex. LC-MS calculated for C₁₅H₁₁BrN₃O (M+H)⁺: m/z=328.0; found 328.0.

### Step 4: N-(3-bromo-2-cyanophenyl)-5-formylpyridine-2-carboxamide

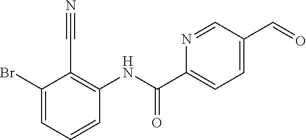 [SMILES: N#Cc1c(Br)cccc1NC(=O)c1ccc(C=O)cn1]

This compound was prepared using a similar procedure as described for Example 3, Step 3 with N-(3-bromo-2-cyanophenyl)-5-vinylpyridine-2-carboxamide replacing 3-methyl-N-(2-methylbiphenyl-3-yl)-5-vinylpyridine-2-carboxamide. LC-MS calculated for C₁₄H₉BrN₃O₂ (M+H)⁺: m/z=329.0; found 329.0.

### Step 5: N-(3-bromo-2-cyanophenyl)-5-{[(2-hydroxyethyl)amino]methyl}pyridine-2-carboxamide

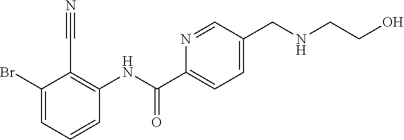 [SMILES: N#Cc1c(Br)cccc1NC(=O)c1ccc(CNCCO)cn1]

This compound was prepared using a similar procedure as described for Example 4 with N-(3-bromo-2-cyanophenyl)-5-formylpyridine-2-carboxamide replacing 5-formyl-3-methyl-N-(2-methylbiphenyl-3-yl)pyridine-2-carboxamide. The reaction mixture was purified by 0 to 12% MeOH in DCM to give the desired product. LC-MS calculated for C₁₆H₁₆BrN₄O₂ (M+H)⁺: m/z=375.0; found 375.0.

### Step 6: N-(2-cyano-3-cyclohex-1-en-1-ylphenyl)-5-{[(2-hydroxyethyl)amino]methyl}pyridine-2-carboxamide

A mixture of N-(3-bromo-2-cyanophenyl)-5-{[(2-hydroxyethyl)amino]methyl}pyridine-2-carboxamide (10 mg, 0.03 mmol), 1,1′-bis(diphenylphosphino)ferrocene]dichloropalladium(II) complexed with DCM (1 mg, 0.002 mmol), sodium carbonate (10 mg), and 2-cyclohex-1-en-1-yl-4,4,5,5-tetramethyl-1,3,2-dioxaborolane (6.66 mg, 0.0320 mmol) in 1,4-dioxane (0.2 mL) and water (0.07 mL) was degassed with N₂ and sealed. It was stirred at 95° C. for 2 h to reach full conversion. The reaction mixture was purified by prep-HPLC (pH=10, acetonitrile/water+NH₄OH) to give the desired product. LC-MS calculated for C₂₂H₂₅N₄O₂ (M+H)⁺: m/z=377.2; found 377.1.

### Example 39: N-(2-cyano-3-piperidin-1-ylphenyl)-5-{[(2-hydroxyethyl)amino]methyl} pyridine-2-carboxamide

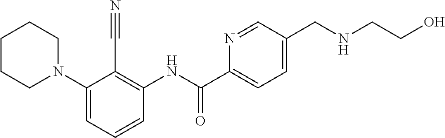 [SMILES: N#Cc1c(NC(=O)c2ccc(CNCCO)cn2)cccc1N1CCCCC1]

A stirred mixture of piperidine (10 μL, 0.1 mmol), N-(3-bromo-2-cyanophenyl)-5-{[(2-hydroxyethyl)amino]methyl}pyridine-2-carboxamide (Example 38, Step 5, 11.2 mg, 0.03 mmol), (2′-aminobiphenyl-2-yl)(chloro)[dicyclohexyl(2′,6′-diisopropoxybiphenyl-2-yl)phosphoranyl]palladium (2.28 mg, 0.003 mmol), and sodium tert-butoxide (20 mg, 0.2 mmol) in 1,4-dioxane (0.083 mL) was heated at 110° C. for 2 h to reach full conversion. The volatiles were removed and the residue was purified by prep-HPLC (pH=2, acetonitrile/water+TFA) to give the desired product as its TFA salt. LC-MS calculated for C₂₁H₂₆N₅O₂ (M+H)⁺: m/z=380.2; found 380.2.

### Example 40: tert-butyl 5-(2-cyano-3-{[(5-{[(2-hydroxyethyl)amino]methyl}pyridin-2-yl)carbonyl]amino}phenyl)-3,6-dihydropyridine-1(2H)-carboxylate

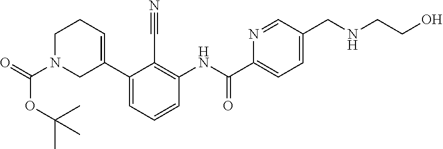 [SMILES: CC(C)(C)OC(=O)N1CCC=C(c2cccc(NC(=O)c3ccc(CNCCO)cn3)c2C#N)C1]

This compound was prepared using a similar procedure as described for Example 38, Step 6 with tert-butyl 5-(4,4,5,5-tetramethyl-1,3,2-dioxaborolan-2-yl)-3,6-dihydropyridine-1(2H)-carboxylate (Combi-Blocks, cat #FM-2863) replacing 2-cyclohex-1-en-1-yl-4,4,5,5-tetramethyl-1,3,2-dioxaborolane. The volatiles were removed and the residue was purified by prep-HPLC (pH=2, acetonitrile/water+TFA) to give the desired product as its TFA salt. LC-MS calculated for C₂₆H₃₂N₅O₄ (M+H)⁺: m/z=478.2; found 478.2.

### Example 41: N-(2-cyano-3-cyclohexylphenyl)-5-{[(2-hydroxyethyl)amino]methyl}pyridine-2-carboxamide

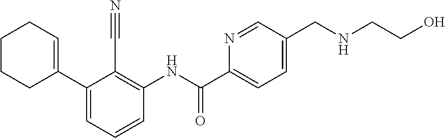 [SMILES: N#Cc1c(NC(=O)c2ccc(CNCCO)cn2)cccc1C1=CCCCC1]

### Step 1: 2-amino-6-cyclohex-1-en-1-ylbenzonitrile

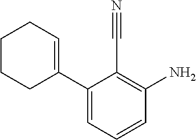 [SMILES: N#Cc1c(N)cccc1C1=CCCCC1]

This compound was prepared using a similar procedure as described for Example 1, Step 1 with 2-cyclohex-1-en-1-yl-4,4,5,5-tetramethyl-1,3,2-dioxaborolane replacing phenylboronic acid, and with 2-amino-6-bromobenzonitrile (Astatech, cat #CL8148) replacing 3-bromo-2-methylaniline. LC-MS calculated for C₁₃H₁₅N₂(M+H)⁺: m/z=199.1; found 199.1.

### Step 2: 2-amino-6-cyclohexylbenzonitrile

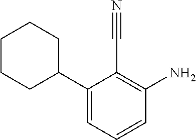 [SMILES: N#Cc1c(N)cccc1C1CCCCC1]

A mixture of 2-amino-6-cyclohex-1-en-1-ylbenzonitrile (100 mg, 0.5 mmol) and 10% Pd/C (53 mg, 0.050 mmol) in methanol (5 mL) was stirred under an atmosphere of H₂ at room temperature for 3 h to reach full conversion. The mixture was filtered through silica gel and concentrated and used for next step without further purification. LC-MS calculated for C₁₃H₁₇N₂ (M+H)⁺: m/z=201.1; found 201.1.

### Step 3: 5-(chloromethyl)picolinoyl chloride

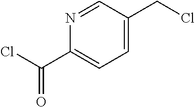 [SMILES: O=C(Cl)c1ccc(CCl)cn1]

1N NaOH solution was added to a THF (1 mL) solution of methyl 5-(bromomethyl) pyridine-2-carboxylate (ArkPharm, cat #AK153186, 20 mg, 0.08 mmol) and the mixture was allowed to stir at rt for 1 h. The mixture was acidified with 1N HCl solution until pH=4, and then the mixture was extracted with DCM. The organic layers were combined, dried and concentrated. To the residue was added 2.0 M oxalyl chloride in DCM (0.10 mL, 0.20 mmol) and N,N-dimethylformamide (0.6 μL, 0.008 mmol) at 0° C. under N₂. The mixture was allowed to stir at rt for 2 h, and then the mixture was concentrated and the residue was used for next step without further purification.

### Step 4: 5-(chloromethyl)-N-(2-cyano-3-cyclohexylphenyl)pyridine-2-carboxamide

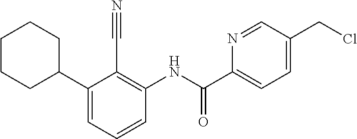 [SMILES: N#Cc1c(NC(=O)c2ccc(CCl)cn2)cccc1C1CCCCC1]

The DCM (1 mL) solution of crude 5-(chloromethyl)picolinoyl chloride was added to a DCM (1 mL) solution of 2-amino-6-cyclohexylbenzonitrile (20 mg, 0.1 mmol) and triethylamine (30 μL, 0.2 mmol) at 0° C. drop-wise. The mixture was allowed to stir at rt for 2 h to reach full conversion. The mixture was concentrated and used for next step without further purification. LC-MS calculated for C₂₀H₂₁C₁N₃O (M+H)⁺: m/z=354.1; found 354.1.

### Step 5: N-(2-cyano-3-cyclohexylphenyl)-5-{[(2-hydroxyethyl)amino]methyl}pyridine-2-carboxamide

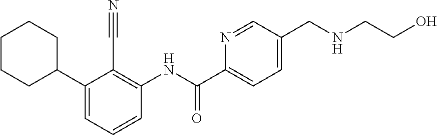 [SMILES: N#Cc1c(NC(=O)c2ccc(CNCCO)cn2)cccc1C1CCCCC1]

A mixture of 5-(chloromethyl)-N-(2-cyano-3-cyclohexylphenyl)pyridine-2-carboxamide (40 mg, 0.1 mmol), ethanolamine (10 μL, 0.2 mmol) and potassium carbonate (41 mg, 0.30 mmol) in N,N-dimethylformamide (0.3 mL, 4 mmol) was stirred at rt for overnight. The mixture was purified by prep-HPLC (pH=2, acetonitrile/water+TFA) to give the desired product as its TFA salt. LC-MS calculated for C₂₂H₂₇N₄O₂ (M+H)⁺: m/z=379.2; found 379.2.

### Example 42: (S)-1-((6-(3-methyl-2-phenylpyridin-4-ylcarbamoyl)pyridin-3-yl)methyl)piperidine-2-carboxylic acid

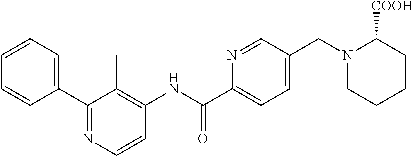 [SMILES: Cc1c(NC(=O)c2ccc(CN3CCCC[C@H]3C(=O)O)cn2)ccnc1-c1ccccc1]

### Step 1: methyl 6-(chlorocarbonyl)nicotinate

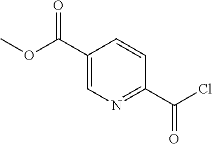

This compound was prepared using a similar procedure as described for Example 41, Step 3 with 5-(methoxycarbonyl)pyridine-2-carboxylic acid (Oakwood Chemical, cat #017196) replacing methyl 5-(bromomethyl) pyridine-2-carboxylate.

### Step 2: methyl 6-{[(2-chloro-3-methylpyridin-4-yl)amino]carbonyl}nicotinate

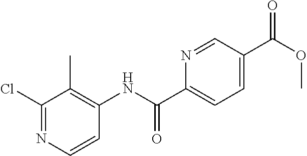 [SMILES: COC(=O)c1ccc(C(=O)Nc2ccnc(Cl)c2C)nc1]

This compound was prepared using a similar procedure as described for Example 41, Step 4 with 2-chloro-3-methylpyridin-4-amine (Astatech, cat #25664) replacing 2-amino-6-cyclohexylbenzonitrile, and with methyl 6-(chlorocarbonyl)nicotinate replacing 5-(chloromethyl)picolinoyl chloride. LC-MS calculated for C₁₄H₁₃C₁N₃O₃(M+H)⁺: m/z=306.1; found 306.1.

### Step 3: methyl 6-{[(3-methyl-2-phenylpyridin-4-yl)amino]carbonyl}nicotinate

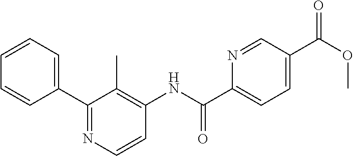 [SMILES: COC(=O)c1ccc(C(=O)Nc2ccnc(-c3ccccc3)c2C)nc1]

Pd-127 (40 mg, 0.06 mmol) was added to the mixture of methyl 6-{[(2-chloro-3-methylpyridin-4-yl)amino]carbonyl}nicotinate (200 mg, 0.6 mmol), phenylboronic acid (132 mg, 1.08 mmol) and cesium carbonate (400 mg, 1 mmol) in 1,4-dioxane (3.69 mL) and water (216 μL). The mixture was stirred at 90° C. for 1 h. After concentration, the mixture was purified by 0 to 50% EtOAc in hexanes to afford the desired product. LC-MS calculated for C₂₀H₁₈N₃O₃ (M+H)⁺: m/z=348.1; found 348.1.

### Step 4: 5-(hydroxymethyl)-N-(3-methyl-2-phenylpyridin-4-yl)pyridine-2-carboxamide

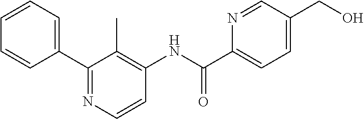 [SMILES: Cc1c(NC(=O)c2ccc(CO)cn2)ccnc1-c1ccccc1]

This compound was prepared using a similar procedure as described for Example 1, Step 3 with methyl 6-{[(3-methyl-2-phenylpyridin-4-yl)amino]carbonyl}nicotinate replacing methyl 6-{[(2-methylbiphenyl-3-yl)amino]carbonyl}nicotinate. LC-MS calculated for C₁₉H₁₈N₃O₂ (M+H)⁺: m/z=320.1; found 320.1.

### Step 5: 5-formyl-N-(3-methyl-2-phenylpyridin-4-yl)pyridine-2-carboxamide

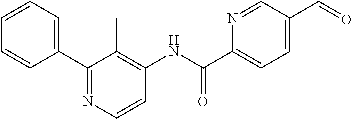 [SMILES: Cc1c(NC(=O)c2ccc(C=O)cn2)ccnc1-c1ccccc1]

This compound was prepared using a similar procedure as described for Example 1, Step 4 with 5-(hydroxymethyl)-N-(3-methyl-2-phenylpyridin-4-yl)pyridine-2-carboxamide replacing 5-(hydroxymethyl)-N-(2-methylbiphenyl-3-yl)pyridine-2-carboxamid. LC-MS calculated for C₁₉H₁₆N₃O₂ (M+H)⁺: m/z=318.1; found 318.1.

### Step 6: (S)-1-((6-(3-methyl-2-phenylpyridin-4-ylcarbamoyl)pyridin-3-yl)methyl)piperidine-2-carboxylic acid

This compound was prepared using a similar procedure as described for Example 1, Step 5 with 5-formyl-N-(3-methyl-2-phenylpyridin-4-yl)pyridine-2-carboxamide replacing 5-formyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₅H₂₇N₄O₃ (M+H)⁺: m/z=431.2; found 431.2.

### Example 43: 5-{[(2-hydroxyethyl)amino]methyl}-N-(3-methyl-2-phenylpyridin-4-yl)pyridine-2-carboxamide

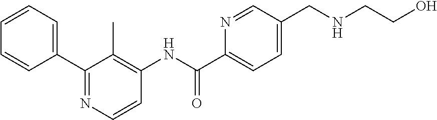 [SMILES: Cc1c(NC(=O)c2ccc(CNCCO)cn2)ccnc1-c1ccccc1]

This compound was prepared using a similar procedure as described for Example 4 with 5-formyl-N-(3-methyl-2-phenylpyridin-4-yl)pyridine-2-carboxamide (Example 42, Step 5) replacing 5-formyl-3-methyl-N-(2-methylbiphenyl-3-yl)pyridine-2-carboxamide. The reaction mixture was purified by prep-HPLC (pH=10, acetonitrile/water+NH₄OH) to give the desired product. LC-MS calculated for C₂₁H₂₃N₄O₂ (M+H)⁺: m/z=363.1; found 363.2.

### Example 44: N-[2-cyano-3-(3,4-dihydro-2H-pyran-5-yl)phenyl]-5-{[(2-hydroxyethyl)amino]methyl}pyridine-2-carboxamide

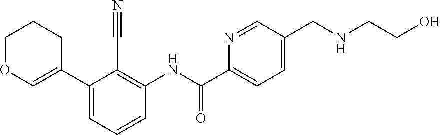 [SMILES: N#Cc1c(NC(=O)c2ccc(CNCCO)cn2)cccc1C1=COCCC1]

This compound was prepared using a similar procedure as described for Example 38, Step 6 with 5-(4,4,5,5-tetramethyl-1,3,2-dioxaborolan-2-yl)-3,4-dihydro-2H-pyran (Combi-Blocks, cat #PN-6040) replacing 2-cyclohex-1-en-1-yl-4,4,5,5-tetramethyl-1,3,2-dioxaborolane. The volatiles were removed, and the residue was purified by prep-HPLC (pH=2, acetonitrile/water+TFA) to give the desired product as its TFA salt. LC-MS calculated for C₂₁H₂₃N₄O₃ (M+H)⁺: m/z=379.2; found 379.2.

### Example 45: N-[3-(2,3-dihydro-1,4-benzodioxin-6-yl)-5-fluoro-2-methylphenyl]-5-{[(2-hydroxyethyl)amino]methyl}pyridine-2-carboxamide

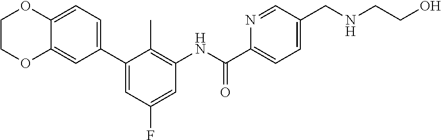 [SMILES: Cc1c(NC(=O)c2ccc(CNCCO)cn2)cc(F)cc1-c1ccc2c(c1)OCCO2]

### Step 1: N-(3-bromo-5-fluoro-2-methylphenyl)-5-(chloromethyl)pyridine-2-carboxamide

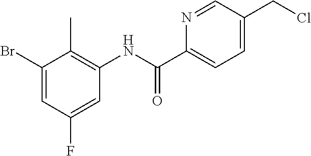 [SMILES: Cc1c(Br)cc(F)cc1NC(=O)c1ccc(CCl)cn1]

This compound was prepared using a similar procedure as described for Example 41, Step 4 with 3-bromo-5-fluoro-2-methylaniline (Combi-Blocks, cat #ST-8934) replacing 2-amino-6-cyclohexylbenzonitrile.

### Step 2: N-(3-bromo-5-fluoro-2-methylphenyl)-5-{[(2-hydroxyethyl)amino]methyl}pyridine-2-carboxamide

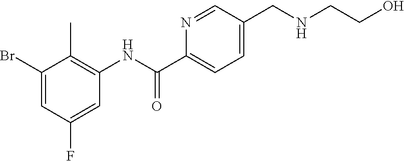 [SMILES: Cc1c(Br)cc(F)cc1NC(=O)c1ccc(CNCCO)cn1]

This compound was prepared using a similar procedure as described for Example 41, Step 5 with N-(3-bromo-5-fluoro-2-methylphenyl)-5-(chloromethyl)pyridine-2-carboxamide replacing 5-(chloromethyl)-N-(2-cyano-3-cyclohexylphenyl)pyridine-2-carboxamide. LC-MS calculated for C₁₆H₁₈BrFN₃O₂(M+H)⁺: m/z=382.1; found 382.0.

### Step 3: N-[3-(2,3-dihydro-1,4-benzodioxin-6-yl)-5-fluoro-2-methylphenyl]-5-{[(2-hydroxyethyl)amino]methyl}pyridine-2-carboxamide

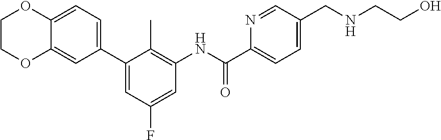 [SMILES: Cc1c(NC(=O)c2ccc(CNCCO)cn2)cc(F)cc1-c1ccc2c(c1)OCCO2]

This compound was prepared using a similar procedure as described for Example 38, Step 6 with 2,3-dihydro-1,4-benzodioxin-6-ylboronic acid (Sigma-Aldrich, cat #635995) replacing 2-cyclohex-1-en-1-yl-4,4,5,5-tetramethyl-1,3,2-dioxaborolane, and with N-(3-bromo-5-fluoro-2-methylphenyl)-5-{[(2-hydroxyethyl)amino]methyl}pyridine-2-carboxamide replacing N-(3-bromo-2-cyanophenyl)-5-{[(2-hydroxyethyl)amino]methyl}pyridine-2-carboxamide. The volatiles were removed, and the residue was purified by prep-HPLC (pH=2, acetonitrile/water+TFA) to give the desired product as its TFA salt. LC-MS calculated for C₂₄H₂₅FN₃O₄(M+H)⁺: m/z=438.2; found 438.1.

### Example 46: N-[2-cyano-3-(4-methyl-3,4-dihydro-2H-1,4-benzoxazin-7-yl)phenyl]-5-{[(2-hydroxyethyl)amino]methyl}pyridine-2-carboxamide

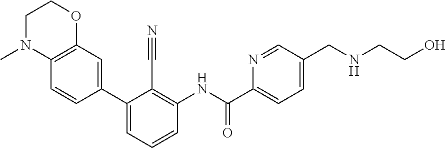 [SMILES: CN1CCOc2cc(-c3cccc(NC(=O)c4ccc(CNCCO)cn4)c3C#N)ccc21]

This compound was prepared using a similar procedure as described for Example 38, Step 6 with 4-methyl-7-(4,4,5,5-tetramethyl-1,3,2-dioxaborolan-2-yl)-3,4-dihydro-2H-1,4-benzoxazine (Maybridge Building Blocks, cat #CC13539) replacing 2-cyclohex-1-en-1-yl-4,4,5,5-tetramethyl-1,3,2-dioxaborolane. The volatiles were removed and the residue was purified by prep-HPLC (pH=2, acetonitrile/water+TFA) to give the desired product as its TFA salt. LC-MS calculated for C₂₅H₂₆N₅O₃ (M+H)⁺: m/z=444.2; found 444.2.

### Example 47: N-[2-cyano-3-(2,3-dihydro-1-benzofuran-6-yl)phenyl]-5-{[(2-hydroxyethyl)amino]methyl}pyridine-2-carboxamide

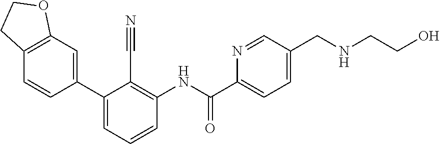 [SMILES: N#Cc1c(NC(=O)c2ccc(CNCCO)cn2)cccc1-c1ccc2c(c1)OCC2]

This compound was prepared using a similar procedure as described for Example 38, Step 6 with (2,3-dihydrobenzofuran-6-yl)boronic acid (ArkPharm, cat #AK143637) replacing 2-cyclohex-1-en-1-yl-4,4,5,5-tetramethyl-1,3,2-dioxaborolane. The volatiles were removed and the residue was purified by prep-HPLC (pH=2, acetonitrile/water+TFA) to give the desired product as its TFA salt. LC-MS calculated for C₂₄H₂₃N₄O₃ (M+H)⁺: m/z=415.2; found 415.2.

### Example 48: N-[2-cyano-3-(2-methyl-2H-indazol-6-yl)phenyl]-5-{[(2-hydroxyethyl)amino]methyl}pyridine-2-carboxamide

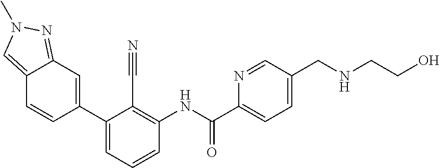 [SMILES: Cn1cc2ccc(-c3cccc(NC(=O)c4ccc(CNCCO)cn4)c3C#N)cc2n1]

This compound was prepared using a similar procedure as described for Example 38, Step 6 with 2-methylindazole-6-boronic acid pinacol ester (Combi-Blocks, cat #PN-9131) replacing 2-cyclohex-1-en-1-yl-4,4,5,5-tetramethyl-1,3,2-dioxaborolane. The volatiles were removed and the residue was purified by prep-HPLC (pH=2, acetonitrile/water+TFA) to give the desired product as its TFA salt. LC-MS calculated for C₂₄H₂₃N₆O₂ (M+H)⁺: m/z=427.2; found 427.2.

### Example 49: N-[2-cyano-3-(1-methyl-1H-indazol-4-yl)phenyl]-5-{[(2-hydroxyethyl)amino]methyl}pyridine-2-carboxamide

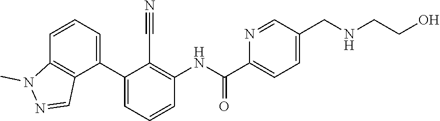 [SMILES: Cn1ncc2c(-c3cccc(NC(=O)c4ccc(CNCCO)cn4)c3C#N)cccc21]

This compound was prepared using a similar procedure as described for Example 38, Step 6 with 1-methyl-1H-indazole-4-boronic acid pinacol ester (Aldrich, cat #725323) replacing 2-cyclohex-1-en-1-yl-4,4,5,5-tetramethyl-1,3,2-dioxaborolane. The volatiles were removed, and the residue was purified by prep-HPLC (pH=2, acetonitrile/water+TFA) to give the desired product as its TFA salt. LC-MS calculated for C₂₄H₂₃N₆O₂ (M+H)⁺: m/z=427.2; found 427.2.

### Example A. PD-1/PD-L1 Homogeneous Time-Resolved Fluorescence (HTRF) Binding Assay

The assays were conducted in a standard black 384-well polystyrene plate with a final volume of 20 μL. Inhibitors were first serially diluted in DMSO and then added to the plate wells before the addition of other reaction components. The final concentration of DMSO in the assay was 1%. The assays were carried out at 25° C. in the PBS buffer (pH 7.4) with 0.05% Tween-20 and 0.1% BSA. Recombinant human PD-L1 protein (19-238) with a His-tag at the C-terminus was purchased from AcroBiosystems (PD1-H5229). Recombinant human PD-1 protein (25-167) with Fc tag at the C-terminus was also purchased from AcroBiosystems (PD1-H5257). PD-L1 and PD-1 proteins were diluted in the assay buffer and 10 μL was added to the plate well. Plates were centrifuged and proteins were preincubated with inhibitors for 40 minutes. The incubation was followed by the addition of 10 μL of HTRF detection buffer supplemented with Europium cryptate-labeled anti-human IgG (PerkinElmer-AD0212) specific for Fc and anti-His antibody conjugated to SureLight®-Allophycocyanin (APC, PerkinElmer-AD0059H). After centrifugation, the plate was incubated at 25° C. for 60 min. before reading on a PHERAstar FS plate reader (665 nm/620 nm ratio). Final concentrations in the assay were −3 nM PD1, 10 nM PD-L1, 1 nM europium anti-human IgG and 20 nM anti-His-Allophycocyanin. IC₅₀ determination was performed by fitting the curve of percent control activity versus the log of the inhibitor concentration using the GraphPad Prism 5.0 software.

Compounds of the present disclosure, as exemplified in the Examples, showed IC₅₀ values in the following ranges: +=IC₅₀≤10 nM; ++=10 nM<IC₅₀≤100 nM; +++=100 nM<IC₅₀≤1000 nM; ++++=IC₅₀>1000 nM

Data obtained for the Example compounds using the PD-1/PD-L1 homogenous time-resolved fluorescence (HTRF) binding assay described in Example A is provided in Table 1.

TABLE 1

| | PD-1/PD-L1 |
|---|---|
| | HTRF |
| Example | IC₅₀ (nM) |
| 1 | ++ |
| 2 | ++ |
| 3 | +++ |
| 4 | ++ |
| 5 | +++ |
| 6 | ++ |
| 7 | ++ |
| 8 | + |
| 9 | ++ |
| 10 | + |
| 11 | ++ |
| 12 | ++ |
| 13 | +++ |
| 14 | ++ |
| 15 | +++ |
| 16 | ++ |
| 17 | ++ |
| 18 | ++ |
| 19 | ++ |
| 20 | ++ |
| 21 | +++ |
| 23 | ++ |
| 24 | +++ |
| 25 | ++ |
| 26 | ++ |
| 27 | ++ |
| 28 | +++ |
| 29 | +++ |
| 30 | +++ |
| 31 | ++ |
| 32 | + |
| 33 | + |
| 34 | ++ |
| 35 | + |
| 36 | ++ |
| 37 | ++ |
| 38 | + |
| 39 | ++++ |
| 40 | +++ |
| 41 | ++ |
| 42 | ++++ |
| 43 | +++ |
| 44 | ++ |
| 45 | + |
| 46 | +++ |
| 47 | ++ |
| 48 | ++ |
| 49 | + |

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A compound of Formula (I′): 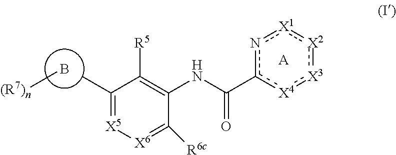
or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
X⁵ is N or CR^{6a};
X⁶ is N or CR^{6b};
ring B is phenyl, 5- or 6-membered heteroaryl, C₃₋₆ cycloalkyl or 5- or 6-membered heterocycloalkyl;
R¹, R², R³, R⁴ and R⁷ are each independently selected from H, halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1, 2, 3, or 4 R^{b} substituents;
R⁵ is C₁₋₄ alkyl or CN;
R^{6a}, R^{6b} and R^{6c} are each independently selected from H, halo, CN, N(C₁₋₆ alkyl)₂, C₁₋₆ alkyl and C₁₋₆ alkoxy;
or two adjacent R⁷ substituents, taken together with the atoms to which they are attached, form a 5-, 6- or 7-membered fused heterocycloalkyl optionally substituted by 1 or 2 R^{q} substituents;
each R^{a} is independently selected from H, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2, 3, 4, or 5 R^{d} substituents;
each R^{d} is independently selected from C₁₋₆ alkyl, C₁₋₆ haloalkyl, halo, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ yl alkyl-C cycloalkyl-C₁₋₄ alkyl-, (5-10 membered heteroaryl)-C₁₋₄ alkyl-, (4-10 membered heterocycloalkyl)-C₁₋₄ alkyl-, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, NR^{e}C(═NOH)NR^{e}R^{e}, NR^{e}C(═NCN)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₆ alkyl, C₁₋₆ haloalkyl, C₆₋₁₀ aryl, 5-10 membered heteroaryl, C₃₋₁₀ cycloalkyl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{d} are each optionally substituted with 1-3 independently selected R substituents;
each R^{b} substituent is independently selected from halo, C₁₋₄ alkyl, C₂₋₄ alkenyl, C₂₋₄ alkynl, C₁₋₄ haloalkyl, C₁₋₄ haloalkoxy, C₆₋₁₀ aryl, C₃₋₁₀ cycloalkyl, 5-10 membered heteroaryl 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₄ alkyl, C₂₋₄ alkenyl, C₂₋₄ alkynyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1-3 independently selected R^{d} substituents;
each R^{c} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2, 3, 4, or 5 R^{f} substituents;
each R^{f} is independently selected from C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, ORB, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2, 3, 4, or 5 Rⁿ substituents;
each Rⁿ is independently selected from C₁₋₄ alkyl, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₁₋₆ haloalkoxy, C₂₋₆ alkenyl, C₂₋₆ alkynyl, C₁₋₄ haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NRS(O)₂R^{o}, NR^{o}S(O)₂NR^{o}R^{o}, and S(O)₂NR^{o}R^{o}, wherein the C₁₋₄ alkyl, C₃₋₁₀ cycloalkyl, 4-7 membered heterocycloalkyl, C₆₋₁₀ aryl, 5-6 membered heteroaryl, C₆₋₁₀ aryl-C₁₋₄ alkyl-, C₃₋₁₀ cycloalkyl-C₁₋₄ alkyl-, (5-6 membered heteroaryl)-C₁₋₄ alkyl-, (4-7 membered heterocycloalkyl)-C₁₋₄ alkyl-, C₁₋₆ haloalkoxy, C₂₋₆ alkenyl, C₂₋₆ alkynyl and C₁₋₄ haloalkyl of Rⁿ are each optionally substituted with 1, 2 or 3 R^{q} substituents;
each R^{g} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1-3 R^{p} substituents;
each R^{p} is independently selected from C₁₋₆ alkyl, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{r}, OR^{r}, SR^{r}, C(O)R^{r}, C(O)NR^{r}R^{r}, C(O)OR^{r}, OC(O)R^{r}, OC(O)NR^{r}R^{r}, NHR^{r}, NR^{r}R^{r}, NR^{r}C(O)R^{r}, NR^{r}C(O)NR^{r}R^{r}, NR^{r}C(O)OR^{r}, C(═NR^{r})NR^{r}R^{r}, NR^{r}C(═NR^{r})NR^{r}R^{r}, NR^{r}C(═NOH)NR^{r}R^{r}, NR^{r}C(═NCN)NR^{r}R^{r}, S(O)R^{r}, S(O)NR^{r}R^{r}, S(O)₂R^{r}, NRS(O)₂R^{r}, NRS(O)₂NR^{r}R^{r} and S(O)₂NR^{r}R^{r}, wherein the C₁₋₆ alkyl, C₁₋₆ haloalkyl, C₁₋₆ haloalkoxy, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{p} are each optionally substituted with 1, 2 or 3 R^{q} substituents;
each R^{e}, R^{o} or R^{r} is independently selected from H, C₁₋₄ alkyl, C₃₋₆ cycloalkyl, C₆₋₁₀ aryl, 5 or 6-membered heteroaryl, 4-6 membered heterocycloalkyl, C₁₋₄ haloalkyl, C₂₋₄ alkenyl, and C₂₋₄ alkynyl, wherein the C₁₋₄ alkyl, C₃₋₆ cycloalkyl, C₆₋₁₀ aryl, 5 or 6-membered heteroaryl, 4-6 membered heterocycloalkyl, C₂₋₄ alkenyl, and C₂₋₄ alkynyl of R^{e}, R^{o} or R^{r} are each optionally substituted with 1, 2 or 3 R^{q} substituents;
each R^{q} is independently selected from OH, CN, —COOH, NH₂, halo, C₁₋₆ haloalkyl, C₁₋₆ alkyl, C₁₋₆ alkoxy, C₁₋₆ alkylthio, phenyl, 5-6 membered heteroaryl, 4-6 membered heterocycloalkyl, C₃₋₆ cycloalkyl, NHR⁸, NR⁸R⁸, and C₁₋₄ haloalkoxy, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkyl, C₁₋₄ alkoxy, C₁₋₄ haloalkyl, C₁₋₄ haloalkoxy, phenyl, C₃₋₁₀ cycloalkyl, 5-6 membered heteroaryl and 4-6 membered heterocycloalkyl; and
each R⁸ is independently C₁₋₆ alkyl; [symbol]
is a single bond or a double bond, wherein ring A includes at least one double bond;
the subscript n is an integer of 1, 2, 3, 4 or 5; and
with the proviso (i) when 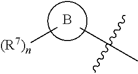
is 2,6-dioxohexahydropyrimidin-1-yl, 2-oxopyrrolidin-1-yl, benzo[d]thiazol-2-yl, 2-amino-4-methyl-5,6-dihydro-1,3-thiazin-4-yl, 4-methyl-6-[4-(morpholine-4-carbonyl)anilino]-5-oxo-pyrazin-2-yl, 5,7-dimethylbenzo[d]oxazol-2-yl, 6-[4-(morpholine-4-carbonyl)phenyl]-7H-pyrrolo[2,3-d]pyrimidin-4-yl, 8-[4-(morpholine-4-carbonyl)anilino]imidazo[1,2-a]pyrazin-6-yl, oxazolo[4,5-b]pyridine-2-yl, or 1-methyl-2-oxo-1,6-naphthridin-3-yl, ring A in Formula (I′) is not 2-pyridyl or 2-pyridyl optionally substituted with halo, methylcarboxy, 1,2,4-triazol-4-yl, 1-piperidinyl, or cyclopropyl;
(ii) when ring B is thiazolo[5,4-b]pyridin-2-yl or 6,7,8,9-tetrahydro-5H-[1,2,4]triazolo[4,3-a]azepin-3-yl, ring A in Formula (I′) is other than 2-quinolyl;
(iii) when ring B is 1-piperazinyl, ring A in Formula (I′) is not 3-(4-benzyloxyphenyl)pyrazolo[1,5-a]pyrimidin-5-yl;
(iv) when ring B is 2-oxo-1,2-dihydropyridin-5-yl, 2-oxo-1,2-dihydropyrazin-5-yl or 6-oxo-1H-pyridazin-3-yl, R² is other than t-butyl;
(v) when 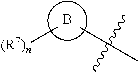
is 3,5-dimethylphenyl, R⁵ is other than 4-amino-1-piperidinyl; and
(vi) the compound is other than 6-((2R,6S)-2,6-dimethylmorpholino)-N-(2-methyl-4′-(trifluoromethoxy)biphenyl-3-yl)pyridazine-3-carboxamide.

2. The compound of claim 1, or a pharmaceutically acceptable salt or a stereoisomer thereof, wherein:
each R^{a} is independently selected from H, CN, C₁₋₆ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2, 3, 4, or 5 R^{d} substituents;
each R^{d} is independently selected from C₁₋₄ alkyl, C₁₋₄haloalkyl, halo, C₃₋₁₀ cycloalkyl, 4-10 membered heterocycloalkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR^{e})NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(o)₂NR^{e}R^{e}, wherein the C₁₋₄ alkyl, C₃₋₁₀ cycloalkyl and 4-10 membered heterocycloalkyl of R^{d} are each further optionally substituted with 1-3 independently selected R^{f} substituents;
each R^{b} substituent is independently selected from halo, C₁₋₄ alkyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NHR^{c}, NR^{c}R^{c}, NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₄ alkyl, C₁₋₄ haloalkyl, C₁₋₄haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1-3 independently selected R^{d} substituents;
each R^{c} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2, 3, 4, or 5 R^{f} substituents;
each R^{f} is independently selected from C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, OR^{g}, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2, 3, 4, or 5 Rⁿ substituents;
each Rⁿ is independently selected from C₁₋₄ alkyl, C₁₋₄haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NR^{o}S(O)₂R^{o}, NR^{o}S(O)₂NR^{o}R^{o}, and S(O)₂NR^{o}R^{o};
each R^{g} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1-3 independently selected R^{p} substituents;
each R^{e}, R^{o} and R^{p} is independently selected from H, C₁₋₄ alkyl, C₃₋₆ cycloalkyl, C₆₋₁₀ aryl, 5 or 6-membered heteroaryl, C₁₋₄ haloalkyl, C₂₋₄ alkenyl, and C₂₋₄ alkynyl, wherein the C₁₋₄ alkyl, C₃₋₆ cycloalkyl, C₆₋₁₀ aryl, 5 or 6-membered heteroaryl, C₂₋₄ alkenyl, and C₂₋₄ alkynyl of R^{e}, R^{o} or R^{p} are each optionally substituted with 1, 2 or 3 R^{q} substituents;
each R^{q} is independently selected from OH, CN, —COOH, NH₂, halo, C₁₋₆ haloalkyl, C₁₋₆ alkyl, C₁₋₆ alkoxy, C₁₋₆ alkylthio, phenyl, 5-6 membered heteroaryl, 4-6 membered heterocycloalkyl, C₃₋₆ cycloalkyl, NHR⁸, NR⁸R⁸, and C₁₋₄ haloalkoxy, wherein the C₁₋₆ alkyl, phenyl, C₃₋₆ cycloalkyl, 4-6 membered heterocycloalkyl, and 5-6 membered heteroaryl of R^{q} are each optionally substituted with halo, OH, CN, —COOH, NH₂, C₁₋₄ alkoxy, C₁₋₄ haloalkyl, C₁₋₄ haloalkoxy, phenyl, C₃₋₁₀ cycloalkyl and 4-6 membered heterocycloalkyl; and
each R⁸ is independently C₁₋₆ alkyl; [symbol]
is a single bond or a double bond, wherein ring A includes at least one double bond;
the subscript n is an integer of 1, 2, 3, 4 or 5; and
with the proviso (i) when 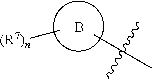
is 2,6-dioxohexahydropyrimidin-1-yl, 2-oxopyrrolidin-1-yl, benzo[d]thiazol-2-yl, 2-amino-4-methyl-5,6-dihydro-1,3-thiazin-4-yl, 4-methyl-6-[4-(morpholine-4-carbonyl)anilino]-5-oxo-pyrazin-2-yl, 5,7-dimethylbenzo[d]oxazol-2-yl, 6-[4-(morpholine-4-carbonyl)phenyl]-7H-pyrrolo[2,3-d]pyrimidin-4-yl, 8-[4-(morpholine-4-carbonyl)anilino]imidazo[1,2-a]pyrazin-6-yl, oxazolo[4,5-b]pyridine-2-yl, or 1-methyl-2-oxo-1,6-naphthridin-3-yl, ring A in Formula (I′) is not 2-pyridyl or 2-pyridyl optionally substituted with halo, methylcarboxy, 1,2,4-triazol-4-yl, 1-piperidinyl, or cyclopropyl;
(ii) when ring B is thiazolo[5,4-b]pyridin-2-yl or 6,7,8,9-tetrahydro-5H-[1,2,4]triazolo[4,3-a]azepin-3-yl, ring A in Formula (I′) is other than 2-quinolyl;
(iii) when ring B is 1-piperazinyl, ring A in Formula (I′) is not 3-(4-benzyloxyphenyl)pyrazolo[1,5-a]pyrimidin-5-yl;
(iv) when ring B is 2-oxo-1,2-dihydropyridin-5-yl, 2-oxo-1,2-dihydropyrazin-5-yl or 6-oxo-1H-pyridazin-3-yl, R² is other than t-butyl;
(v) when 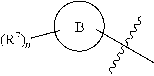
is 3,5-dimethylphenyl, R⁵ is other than 4-amino-1-piperidinyl; and
(vi) the compound is other than 6-((2R,6S)-2,6-dimethylmorpholino)-N-(2-methyl-4′-(trifluoromethoxy)biphenyl-3-yl)pyridazine-3-carboxamide.

3. The compound of claim 1 having Formula (I): 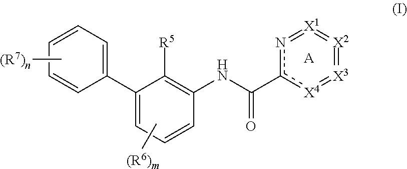
or a pharmaceutically acceptable salt or a stereoisomer thereof, wherein:
X¹ is N or CR¹;
X² is N or CR²;
X³ is N or CR³;
X⁴ is N or CR⁴;
wherein no more than two of X¹, X², X³ and X⁴ are simultaneously N;
R¹, R², R³, R⁴ and R⁷ are each independently selected from H, halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R¹, R², R³, R⁴ and R⁷ are each optionally substituted with 1, 2, 3, or 4 R^{b} substituents;
R⁵ is C₁₋₄ alkyl or CN;
each R⁶ is independently selected from H, halo, CN, N(C₁₋₆ alkyl)₂, C₁₋₆ alkyl and C₁₋₆ alkoxy H, C₁₋₄ alkyl, C₃₋₆ cycloalkyl, C₂₋₄ alkenyl, C₂₋₄ alkynyl, halo, CN, OH, C₁₋₄ alkoxy, C₁₋₄ haloalkyl, C₁₋₄ haloalkoxy, NH₂, —NH—C₁₋₄ alkyl, —N(C₁₋₄ alkyl)₂, NHOR¹⁰, C(O)R¹⁰, C(O)NR¹⁰R¹⁰, C(O)OR¹⁰, OC(O)R¹⁰, OC(O)NR¹⁰R¹⁰, NR¹⁰C(O)R¹⁰, NR¹⁰C(O)OR¹⁰, NR¹⁰C(O)NR¹⁰R¹⁰, C(═NR¹⁰)R¹⁰, C(═NR¹⁰)NR¹⁰R¹⁰, NR¹⁰C(═NR¹⁰)NR¹⁰R¹⁰, NR¹⁰S(O)R¹⁰, NR¹⁰S(O)₂R¹⁰, NR¹⁰S(O)₂NR¹⁰R¹⁰, S(O)R¹⁰, S(O)NR¹⁰R¹⁰, S(O)₂R¹⁰, and S(O)₂NR¹⁰R¹⁰, wherein each R¹⁰ is independently H or C₁₋₄ alkyl optionally substituted with 1 or 2 groups independently selected from halo, OH, CN and C₁₋₄ alkoxy, and wherein the C₁₋₄ alkyl, C₃₋₄ cycloalkyl, C₂₋₄ alkenyl and C₂₋₄ alkynyl of R⁶ are each optionally substituted with 1 or 2 substituents independently selected from halo, OH, CN, C₁₋₄ alkyl and C₁₋₄ alkoxy;
or two adjacent R⁷ substituents, taken together with the carbon atoms to which they are attached, form a 5-, 6- or 7-membered fused heterocycloalkyl optionally substituted by 1 or 2 R^{q} substituents;
each R^{a} is independently selected from H, CN, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{a} are each optionally substituted with 1, 2, 3, 4, or 5 R^{d} substituents;
each R^{d} is independently selected from C₁₋₄ alkyl, C₁₋₄haloalkyl, halo, C₃₋₁₀ cycloalkyl, 4-10 membered heterocycloalkyl, CN, NH₂, NHOR^{e}, OR^{e}, SR^{e}, C(O)R^{e}, C(O)NR^{e}R^{e}, C(O)OR^{e}, OC(O)R^{e}, OC(O)NR^{e}R^{e}, NHR^{e}, NR^{e}R^{e}, NR^{e}C(O)R^{e}, NR^{e}C(O)NR^{e}R^{e}, NR^{e}C(O)OR^{e}, C(═NR^{e})NR^{e}R^{e}, NR^{e}C(═NR)NR^{e}R^{e}, S(O)R^{e}, S(O)NR^{e}R^{e}, S(O)₂R^{e}, NR^{e}S(O)₂R^{e}, NR^{e}S(O)₂NR^{e}R^{e}, and S(O)₂NR^{e}R^{e}, wherein the C₁₋₄ alkyl, C₃₋₁₀ cycloalkyl and 4-10 membered heterocycloalkyl of R^{d} are each further optionally substituted with 1-3 independently selected R^{q} substituents;
each R^{b} substituent is independently selected from halo, C₁₋₄ alkyl, C₁₋₄ haloalkyl, C₁₋₄ haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, CN, OH, NH₂, NO₂, NHOR^{c}, OR^{c}, SR^{c}, C(O)R^{c}, C(O)NR^{c}R^{c}, C(O)OR^{c}, OC(O)R^{c}, OC(O)NR^{c}R^{c}, C(═NR^{c})NR^{c}R^{c}, NR^{c}C(═NR^{c})NR^{c}R^{c}, NHR^{c}, NR^{c}R^{c}NR^{c}C(O)R^{c}, NR^{c}C(O)OR^{c}, NR^{c}C(O)NR^{c}R^{c}, NR^{c}S(O)R^{c}, NR^{c}S(O)₂R^{c}, NR^{c}S(O)₂NR^{c}R^{c}, S(O)R^{c}, S(O)NR^{c}R^{c}, S(O)₂R^{c} and S(O)₂NR^{c}R^{c}; wherein the C₁₋₄ alkyl, C₁₋₄ haloalkyl, C₁₋₄haloalkoxy, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{b} are each further optionally substituted with 1-3 independently selected R^{d} substituents;
each R^{c} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{c} are each optionally substituted with 1, 2, 3, 4, or 5 R^{f} substituents;
each R^{f} is independently selected from C₁₋₄ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, (4-10 membered heterocycloalkyl)-C₁₋₄ alkyl-, halo, CN, NHOR^{g}, ORB, SR^{g}, C(O)R^{g}, C(O)NR^{g}R^{g}, C(O)OR^{g}, OC(O)R^{g}, OC(O)NR^{g}R^{g}, NHR^{g}, NR^{g}R^{g}, NR^{g}C(O)R^{g}, NR^{g}C(O)NR^{g}R^{g}, NR^{g}C(O)OR^{g}, C(═NR^{g})NR^{g}R^{g}, NR^{g}C(═NR^{g})NR^{g}R^{g}, S(O)R^{g}, S(O)NR^{g}R^{g}, S(O)₂R^{g}, NR^{g}S(O)₂R^{g}, NR^{g}S(O)₂NR^{g}R^{g}, and S(O)₂NR^{g}R^{g}; wherein the C₁₋₄ alkyl, C₁₋₄ haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl- of R^{f} are each optionally substituted with 1, 2, 3, 4, or 5 Rⁿ substituents;
each Rⁿ is independently selected from C₁₋₄ alkyl, C₁₋₄haloalkyl, halo, CN, R^{o}, NHOR^{o}, OR^{o}, SR^{o}, C(O)R^{o}, C(O)NR^{o}R^{o}, C(O)OR^{o}, OC(O)R^{o}, OC(O)NR^{o}R^{o}, NHR^{o}, NR^{o}R^{o}, NR^{o}C(O)R^{o}, NR^{o}C(O)NR^{o}R^{o}, NR^{o}C(O)OR^{o}, C(═NR^{o})NR^{o}R^{o}, NR^{o}C(═NR^{o})NR^{o}R^{o}, S(O)R^{o}, S(O)NR^{o}R^{o}, S(O)₂R^{o}, NRS(O)₂R^{o}, NRS(O)₂NR^{o}R^{o}, and S(O)₂NR^{o}R^{o};
each R^{g} is independently selected from H, C₁₋₆ alkyl, C₁₋₄haloalkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl-, and (4-10 membered heterocycloalkyl)-C₁₋₄ alkyl-, wherein the C₁₋₆ alkyl, C₂₋₆ alkenyl, C₂₋₆ alkynyl, C₆₋₁₀ aryl, C₃₋₁₀ cycloalkyl, 5-10 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-10 membered heteroaryl)-C₁₋₄ alkyl- and (4-10 membered heterocycloalkyl)-C₁₋₄ alkyl- of R^{g} are each optionally substituted with 1-3 independently selected R^{p} substituents;
each R^{o}, R^{o} or R^{p} is independently selected from H, C₁₋₄ alkyl, C₃₋₆ cycloalkyl, C₆₋₁₀ aryl, 5 or 6-membered heteroaryl, C₁₋₄ haloalkyl, C₂₋₄ alkenyl, and C₂₋₄ alkynyl, wherein the C₁₋₄ alkyl, C₃₋₆ cycloalkyl, C₆₋₁₀ aryl, 5 or 6-membered heteroaryl, C₂₋₄ alkenyl, and C₂₋₄ alkynyl of R^{o}, R^{o} or R^{p} are each optionally substituted with 1, 2 or 3 R^{q} substituents;
each R^{q} is independently selected from OH, CN, —COOH, NH₂, halo, C₁₋₄ alkyl, C₁₋₄ alkoxy, C₁₋₄ alkylthio, phenyl, 5-6 membered heteroaryl, C₃₋₆ cycloalkyl, NHR⁸, NR⁸R⁸, and C₁₋₄ haloalkoxy, wherein the C₁₋₄ alkyl, phenyl and 5-6 membered heteroaryl of R^{q} are each optionally substituted with OH, CN, —COOH, NH₂, C₁₋₄ alkoxy, C₃₋₁₀ cycloalkyl and 4-, 5-, or 6-membered heterocycloalkyl; and
each R⁸ is independently C₁₋₆ alkyl;
the subscript n is an integer of 1, 2, 3, 4 or 5;
the subscript m is an integer of 1, 2 or 3; and [symbol]
is a single bond or a double bond, wherein ring A includes at least one double bond;
with the proviso that the compound is other than 6-((2R,6S)-2,6-dimethylmorpholino)-N-(2-methyl-4′-(trifluoromethoxy)biphenyl-3-yl)pyridazine-3-carboxamide.

4. (canceled)

5. The compound of claim 1, having Formula (II): 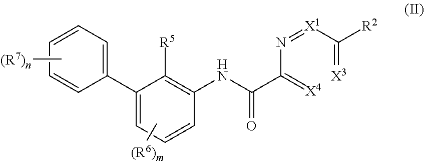
wherein R² is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R² are each optionally substituted with 1, 2, 3, or 4 R^{b} substituents, or a pharmaceutically acceptable salt or a stereoisomer thereof.

6.-9. (canceled)

10. The compound of claim 1, or a pharmaceutically acceptable salt or a stereoisomer thereof, wherein the moiety 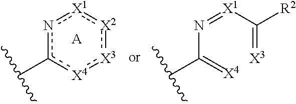
is selected from:

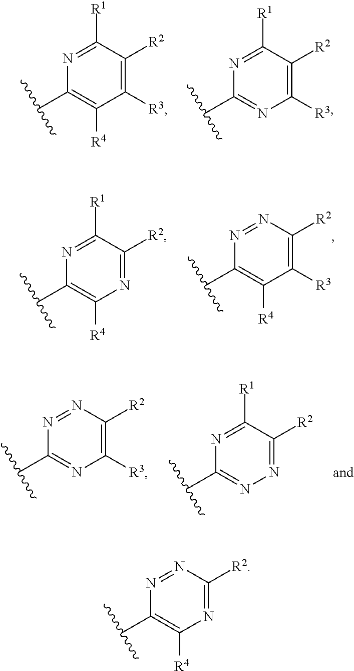

11. The compound of claim 1, having Formula (VII): 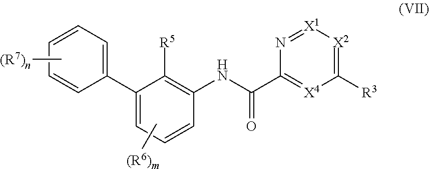
wherein R³ is halo, C₁₋₆ alkyl, C₂₋₆ alkenyl, C₂₋₆ alkynyl, C₁₋₆ haloalkyl, C₁₋₆ haloalkoxy, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, (4-10 membered heterocycloalkyl)-C₁₋₄ alkyl-, CN, NO₂, OR^{a}, SR^{a}, NHOR^{a}, C(O)R^{a}, C(O)NR^{a}R^{a}, C(O)OR^{a}, OC(O)R^{a}, OC(O)NR^{a}R^{a}, NHR^{a}, NR^{a}R^{a}, NR^{a}C(O)R^{a}, NR^{a}C(O)OR^{a}, NR^{a}C(O)NR^{a}R^{a}, C(═NR^{a})R^{a}, C(═NR^{a})NR^{a}R^{a}, NR^{a}C(═NR^{a})NR^{a}R^{a}, NR^{a}S(O)R^{a}, NR^{a}S(O)₂R^{a}, NR^{a}S(O)₂NR^{a}R^{a}, S(O)R^{a}, S(O)NR^{a}R^{a}, S(O)₂R^{a}, and S(O)₂NR^{a}R^{a}, wherein the C₁₋₆ alkyl, C₂₋₆ alkenyl, C₂₋₆ alkynyl, C₆₋₁₀ aryl, C₃₋₁₀ cycloalkyl, 5-14 membered heteroaryl, 4-10 membered heterocycloalkyl, C₆₋₁₀ aryl-C₁₋₄ alkyl-, C₃₋₁₀ cycloalkyl-C₁₋₄ alkyl-, (5-14 membered heteroaryl)-C₁₋₄ alkyl-, and (4-10 membered heterocycloalkyl)-C₁₋₄ alkyl- of R¹ are each optionally substituted with 1, 2, 3, or 4 R^{b} substituents, or a pharmaceutically acceptable salt or a stereoisomer thereof.

12. The compound of claim 1, or a pharmaceutically acceptable salt or a stereoisomer thereof, wherein the moiety 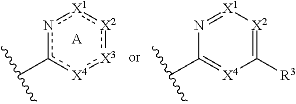
is selected from:

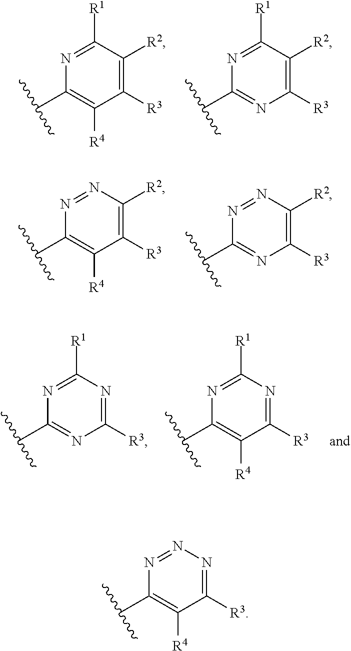

13-16. (canceled)

17. The compound of claim 1, or a pharmaceutically acceptable salt or a stereoisomer thereof, wherein R¹, R³ and R⁴, when applicable, are each independently selected from H, C₁₋₆ alkyl and halo.

18. The compound of claim 11, or a pharmaceutically acceptable salt or a stereoisomer thereof, wherein R¹, R² and R⁴, when applicable, are each independently selected from H, C₁₋₆ alkyl and halo.

19. The compound of claim 1, or a pharmaceutically acceptable salt or a stereoisomer thereof, wherein R² is —CH₂—R^{b}.

20. (canceled)

21. The compound of claim 11, or a pharmaceutically acceptable salt or a stereoisomer thereof, wherein R³ is —CH₂—R^{b}.

22. (canceled)

23. The compound of claim 1, or a pharmaceutically acceptable salt or a stereoisomer thereof, wherein R² is 2-hydroxyethylaminomethyl, 2-carboxypiperidin-1-ylmethyl, (S)-2-carboxypiperidin-1-ylmethyl, (R)-2-carboxypiperidin-1-ylmethyl or (3-cyanobenzyl)oxy.

24. The compound of claim 1, or a pharmaceutically acceptable salt or a stereoisomer thereof, wherein R³ is 2-hydroxyethylaminomethyl, 2-carboxypiperidin-1-ylmethyl, (S)-2-carboxypiperidin-1-ylmethyl, (R)-2-carboxypiperidin-1-ylmethyl or (3-cyanobenzyl)oxy.

25.-27. (canceled)

28. The compound of claim 1, wherein the compound is selected from:
(2S)-1-[(2-{[(2-methylbiphenyl-3-yl)amino]carbonyl}pyridin-4yl)methyl]piperidine-2-carboxylic acid;
4-{[(2-hydroxyethyl)amino]methyl}-N-(2-methylbiphenyl-3-yl)pyridine-2-carboxamide;
N-(2-methylbiphenyl-3-yl)-6-(pyridin-3-ylmethoxy)pyrimidine-4-carboxamide;
4-cyano-N-(2-methylbiphenyl-3-yl)pyridine-2-carboxamide;
N-(2-methylbiphenyl-3-yl)pyrazine-2-carboxamide;
N-(2-methylbiphenyl-3-yl)pyrimidine-4-carboxamide;
N-(2-methylbiphenyl-3-yl)pyrimidine-2-carboxamide;
N-(2-methylbiphenyl-3-yl)pyridazine-3-carboxamide; and
N-(2-methylbiphenyl-3-yl)-2-(pyridin-3-ylmethoxy)pyrimidine-4-carboxamide;
or a pharmaceutically acceptable salt thereof.

29. The compound of claim 1, wherein the compound is selected from:
N-(2-cyano-3-cyclohex-1-en-1-ylphenyl)-5-{[(2-hydroxyethyl)amino]}pyridine-2-carboxamide;
N-(2-cyano-3-piperidin-1-ylphenyl)-5-{[(2-hydroxyethyl)amino]methyl}pyridine-2-carboxamide;
tert-butyl 5-(2-cyano-3-{[(5-{[(2-hydroxyethyl)amino]methyl}pyridin-2-yl)carbonyl]amino}phenyl)-3,6-dihydropyridine-1(2H)-carboxylate;
N-(2-cyano-3-cyclohexylphenyl)-5-{[(2-hydroxyethyl)amino]methyl}pyridine-2-carboxamide;
(S)-1-((6-(3-methyl-2-phenylpyridin-4-ylcarbamoyl)pyridin-3-yl)methyl)piperidine-2-carboxylic acid;
5-{[(2-hydroxyethyl)amino]methyl}-N-(3-methyl-2-phenylpyridin-4-yl)pyridine-2-carboxamide;
N-[2-cyano-3-(3,4-dihydro-2H-pyran-5-yl)phenyl]-5-{[(2-hydroxyethyl)amino] methyl}pyridine-2-carboxamide; and
N-[3-(2,3-dihydro-1,4-benzodioxin-6-yl)-5-fluoro-2-methylphenyl]-5-{[(2-hydroxyethyl)amino]methyl}pyridine-2-carboxamide;
or a pharmaceutically acceptable salt thereof.

30. A pharmaceutical composition comprising a compound of claim 1, or a pharmaceutically acceptable salt or a stereoisomer thereof, and a pharmaceutically acceptable carrier or excipient.

31. A method of inhibiting PD-1/PD-L1 interaction, said method comprising administering to a patient a compound of claim 1, or a pharmaceutically acceptable salt or a stereoisomer thereof.

32. A method of treating a disease or disorder associated with PD-1/PD-L1 interaction, said method comprising administering to a patient in need thereof a therapeutically effective amount of a compound of claim 1, or a pharmaceutically acceptable salt or a stereoisomer thereof.

33. (canceled)

34. A method of enhancing, stimulating, modulating and/or increasing the immune response in a patient, said method comprising administering to the patient in need thereof a therapeutically effective amount of claim 1, or a pharmaceutically acceptable salt thereof or a stereoisomer thereof.

35. A method of inhibiting growth, proliferation, or metastasis of cancer cells in a patient, said method comprising administering to the patient in need thereof a therapeutically effective amount of a compound of claim 1, or a pharmaceutically acceptable salt thereof or a stereoisomer thereof.
